(19) United States
(12) Patent Application Publication
(10) Pub. No.: US 2008/0004230 A1
(43) Pub. Date: Jan. 3, 2008

(54) SAM Riboswitch and Uses Thereof

(76) Inventors: Robert T. Batey, Boulder, CO (US); Rebecca K. Montange, Boulder, CO (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(21) Appl. No.: 11/676,985

(22) Filed: Feb. 20, 2007

Related U.S. Application Data
(60) Provisional application No. 60/774,489, filed on Feb. 17, 2006.

(51) Int. Cl. A61K 48/00; A61P 43/00; C07H 21/04; C12N 15/87; C12Q 1/68

(52) U.S. Cl. 514/44; 435/471; 435/6; 536/22.1

(57) ABSTRACT

Embodiments of the present invention provide for SAM-I riboswitches and analogs thereof, and methods for using the same. In certain embodiments of the present invention, test compounds are identified that associate with SAM-I riboswitches.

### CROSS-REFERENCE TO RELATED APPLICATIONS

This application claims benefit under 35 U.S.C. §119(e) of provisional U.S. Application No. 60/774,489, filed Feb. 17, 2006 and is hereby incorporated herein by reference in its entirety for all purposes.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant No. R-01 GM073850-01 awarded by the National Institutes of Health.

### FIELD OF THE INVENTION

The present invention relates to compositions and methods of use thereof related to SAM-I riboswitch.

### BACKGROUND OF THE INVENTION

Riboswitches are regulatory elements found within the 5′-untranslated regions (5′-UTRs) of many bacterial mRNAs. Riboswitches control gene expression in a cis-fashion through their ability to directly bind a specific small molecule metabolite. Ligand recognition is effected by the first domain of the riboswitch, termed the aptamer domain while the second, the expression platform, transduces the binding event into a regulatory switch. The switch includes an RNA element that can adapt to one of two mutually exclusive secondary structures. One of these structures is a signal for gene expression to be “on” and the other conformation turns the gene “off” (example in FIG. 1). In Bacillus subtilis and other gram positive bacteria, it is believed riboswitches control greater than 2% of all genes, many of which are important for key pathways controlling the amino acid, nucleotide and cofactor metabolism.

Riboswitch aptamer domains are controlled by a diverse set of metabolites. In one example bacteria, amino acid metabolism in various Bacillus species is controlled by three known riboswitches: glycine, lysine and S-adenosylmethionine (SAM). Each has a distinct aptamer domain that has evolved to specifically recognize a specific ligand. Currently, there are two known distinct SAM riboswitches, one of which is dominant in gram positive bacteria, SAM-I, and one dominant in gram negative bacteria, SAM-II. The SAM-I riboswitch, which regulates methionine uptake and synthesis as well as SAM synthesis, contains a secondary structure comprised of four stem-loops surrounding a four-way junction motif In the almost 300 individual SAM-I riboswitches that have been identified, a number of nucleotides within and surrounding the junction are highly phylogenetically conserved (FIG. 1), including a consensus kink-turn motif and genetically-validated pseudoknot structure. Characterization of the binding of SAM analogs has indicated that this RNA recognizes substantially every feature of the ligand, although the reactive methyl group indirectly.

A need exist to better control bacterial growth and generate effective treatments against bacterial infections. Embodiments of the present invention fulfill this need.

### SUMMARY OF THE INVENTION

One aspect of the present invention provides for methods of identifying a compound that associates with a SAM-I riboswitch including modeling at least a portion of the atomic structure depicted in FIGS. 2A and 2b with a test compound; and determining the interaction between the test compound and the SAM-I riboswitch structure.

Certain embodiments herein concern crystalline atomic structures of SAM-I riboswitches. In accordance with the methods, the structures may also be used for modeling and assessing the interaction of a riboswitch with a binding ligand.

In other embodiments herein, a compound may be identified that associates with the SAM-I riboswitch and reduces bacterial gene expression or associates with the SAM-I riboswitch and induces bacterial gene expression. In accordance with these embodiments, atomic coordinates of the atomic structure can include at least a portion of the atomic coordinates listed in Table 1 for atoms depicted in FIG. 2A or 2b wherein said association determination step can include determining a minimum interaction energy, a binding constant, a dissociation constant, or a combination thereof, for the test compound in the model of the SAM-I riboswitch. In some particular embodiments, an association determination step can include determining the interaction of the test compound with a nucleotide of SAM-I riboswitch including A6, U6, G11, A45, C47, U57, G58, A86, U87, or a combination thereof In other embodiments, an association determination step can include determining the interaction of the test compound with an S-adenosyl-methionine moiety including a ribose sugar, a methionine side chain, a sulfur moiety, an adenine moiety or combination thereof. Alternatively, in a more particular embodiment, the association determination step can include determining the interaction of the test compound with a nucleotide of SAM-I riboswitch depicted in FIG. 2A or 2B including A45, G11, C44, G58 and U57 or a combination thereof Other embodiments contemplated herein include an association determination step of determining the interaction of the test compound with a P3 helix region of the SAM-I riboswitch. Yet other embodiments contemplated herein can include an association determination step including determining the interaction of the test compound within a pocket created between a P1 and P3 helices of the SAM-I riboswitch. Further embodiments concern an association determination step including determining the interaction of the test compound with a minor groove of a P1 and P3 helices of the SAM-I riboswitch.

Bacterial cells contemplated of use in the methods and compositions herein include, but are not limited to, Staphylococcus spp., Bacillus spp., Listeria spp., Clostridia spp., Streptomyces spp., Thermoanaerobacteria spp. and a combination thereof.

In certain embodiments, a SAM-I riboswitch disclosed herein can include one or more of the nucleotides listed in “Tertiary contacts” section of Table 2 where the nucleotide can be modified. In certain embodiments, the one or more modified nucleotides are selected from the group consisting of A45, G11, C44, G58 and U57. In particular embodiments, the modified nucleotide of the SAM-I riboswitch can increase gene expression in a bacterial cell. For example, a test compound that contains a modified nucleotide may induce expression of a gene that is deleterious to a bacterial cell. In other embodiments, the modified nucleotide can decrease gene expression in a cell. For example, a test compound that contains a modified nucleotide may reduce expression of a gene that is necessary for survival of a bacterial cell. In certain particular embodiments, the modified nucleotide decreases sulfur production in a cell.

Embodiments of the present invention concern a test compound that associates with at least a portion of the SAM-I riboswitch atomic structure depicted in at least one of FIG. 2A or FIG. 2B. In accordance with these embodiments, the association can include association with at least one of nucleotides A45, G11, C44, G58 and U57, wherein the composition is capable of modifying the SAM-I riboswitch activity of a bacterial organism by either inducing or reducing gene expression.

Certain embodiments concern compositions including, all of the 80 percent or more conserved nucleotides of the SAM-I riboswitch depicted in FIG. 1 left and 80% or greater, or 90% or greater or 95% or greater of the nucleotides depicted outside of the conserved region. One particular embodiment includes a composition of all 80 percent or more conserved nucleotides of the SAM-I riboswitch depicted in FIG. 1 left and all of the nucleotides depicted outside of the conserved region.

In one embodiment, the atomic coordinates of the atomic structure comprise the atomic coordinates listed in Table 1 for atoms depicted in FIGS. 2A and 2b.

Yet in another embodiment, the interaction determination step can include determining a minimum interaction energy, a binding constant, a dissociation constant, or a combination thereof, for the test compound in the model of the SAM-I riboswitch.

Still in other embodiments, the interaction determination step and test compound identification can include determining the interaction of the test compound with a nucleotide of SAM-I riboswitch comprising A6, U6, G11, A45, C47, U57, G58, A86, U87, or a combination thereof Within this embodiment, the interaction determination step can include determining the interaction of the test compound with a nucleotide of SAM-I riboswitch comprising A45, G11, C44, G58 and U57, or a combination thereof In addition, the test compound that effectively interacts with one or more of the above mentioned nucleotides can be identified and expanded for use in targeting bacterial organisms disclosed herein.

Another aspect of the present invention provides, a method of regulating a gene in a cell by modulating an mRNA, said method comprising administering a SAM-I riboswitch modulating compound to the cell to modulate the SAM-I riboswitch activity of the mRNA. In certain embodiments, the gene expression is stimulated, while in other embodiments the gene expression is inhibited. Within certain embodiments where the gene expression is inhibited, the SAM-I riboswitch modulating compound forms a complex with the SAM-I riboswitch, thereby preventing the mRNA from forming an antiterminator element.

Another aspect of the present invention provides a SAM-I riboswitch in which one or more of the nucleotides listed in “Tertiary contacts” section of Table 2 is modified, e.g., replaced with another nucleotide. Alternatively, certain embodiments include a compound that associates with one or more of the contact nucleotides and modulates the activity of the SAM-1 riboswitch. In one particular embodiment, a compound capable of associating with one or more of the contact nucleotides may be capable of reducing sulfur metabolism in an organism having a SAM-I or SAM-I like riboswitch. In accordance with these embodiments, compounds of the present invention may be used to reduce infection caused by, or as a treatment for infection caused by an organism contemplated herein. In certain embodiments target organisms include bacteria. Bacteria contemplated herein include, but are not limited to, Staphylococcus spp., Bacillus spp., Listeria spp., Clostridia spp., Streptomyces spp., Thermoanaerobacteria spp. and a combination thereof.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIG. 1 (SEQ ID NO:2) represents a schematic of switching by a SAM-I riboswitch.

FIGS. 2A (SEQ ID NO:3) and 2B represents a schematic of the secondary structure of the SAM-I riboswitch and a global view of a three-dimensional structure of the SAM-I riboswitch, respectively.

FIG. 3 represents tertiary architecture of the pseudoknot.

FIGS. 4A and 4B represent schematics of S-adenosyl-methionine recognition by the SAM-I riboswitch.

FIG. 5 represents a schematic of direct interactions between the P1 and P3 helices.

FIG. 6 represents an exemplary crystal structure of SAM-I and data collection and model refinement.

### DEFINITIONS

As used herein, “a” or “an” may mean one or more than one of an item.

### DETAILED DESCRIPTION

In the following sections, various exemplary compositions and methods are described in order to detail various embodiments of the invention. It will be obvious to one skilled in the art that practicing the various embodiments does not require the employment of all or even some of the specific details outlined herein, but rather that molecules, test compounds, samples, concentrations, times and other specific details may be modified through routine experimentation. In some cases, well known methods or components have not been included in the description.

Embodiments of the present invention provide for compositions and methods concerning SAM-I riboswitch and SAM-I riboswitch-like molecules.

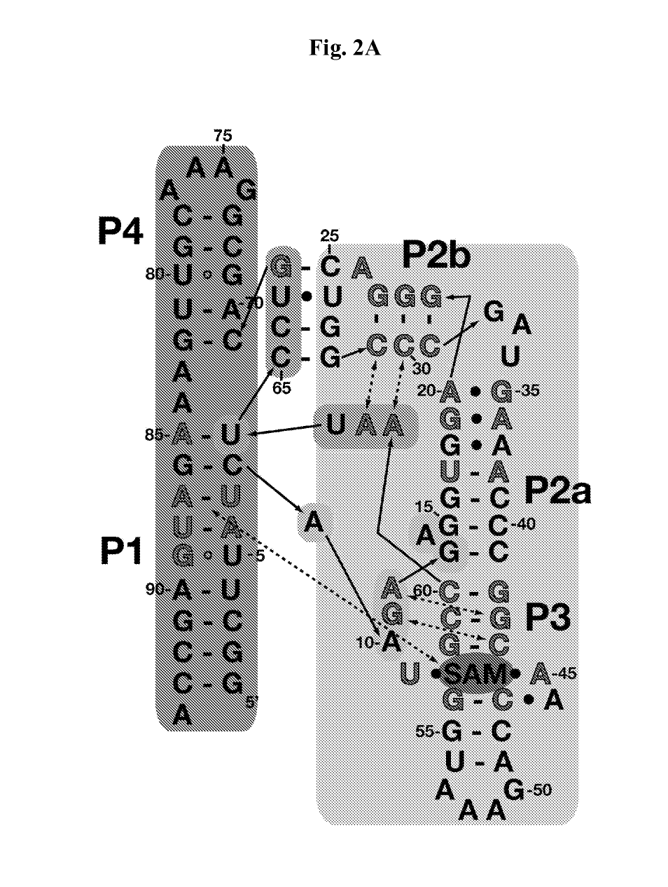

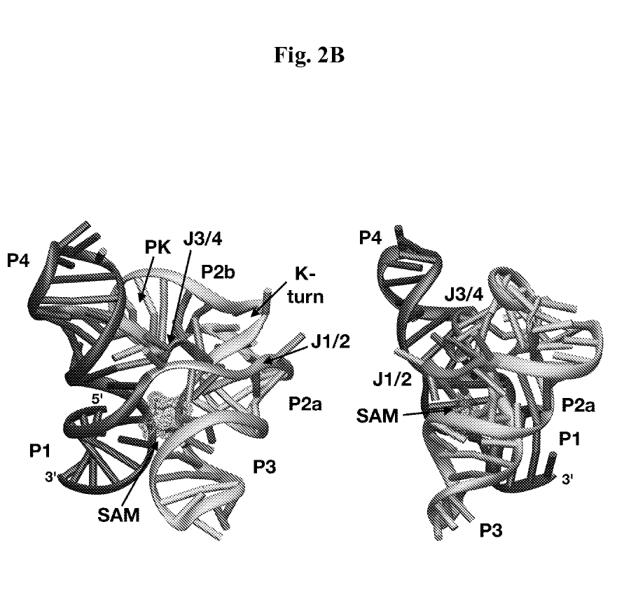

In certain embodiments herein, the aptamer domain of the SAM-I riboswitch that controls the metFH2 operon in Thermoanerobacter tengcogensis was used as a template for construction of an RNA that could be crystallized in the presence of SAM (S-adenylsylmethionine). A series of constructs were made that encompassed all of the nucleotides that were >95% conserved across phylogeny and preserved the integrity of the four-way junction motif Out of ˜30 constructs tested, one (FIG. 2A) crystallized reproducibly, yielding crystals of P4₃2₁2 space group and diffracted to 2.8 Å resolution. Phase information was obtained using iridium hexammine, using a two wavelength MAD experiment. This yielded a readily interpretable electron density map, allowing for all 94 nucleotides to be built into the model along with the S-adenosylmethionine ligand. The final model has excellent geometry with a final Rₓₜₐₗ of 26.7% and R_{free} of 28.8% (Table 1).

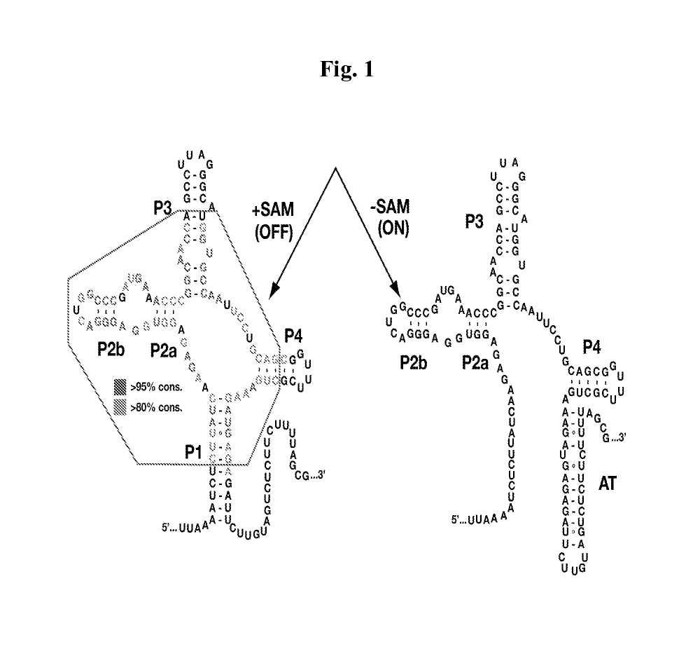

Ligand recognition is effected by the first domain of the riboswitch, termed the aptamer domain while the second, the expression platform, transduces the binding event into a regulatory switch. The switch comprises an RNA element that can adopt one of two mutually exclusive secondary structures in which one signal for gene expression to be on and the other conformation turns the gene off (FIG. 1). In B. subtilis and other gram positive bacteria, riboswitches control>2% of all genes, many of which are important for key pathways controlling the amino acid, nucleotide and cofactor metabolism.

Riboswitch aptamer domains are controlled by a diverse set of metabolites. Amino acid metabolism in various Bacillus species is controlled by three known riboswitches: glycine, lysine and S-adenosylmethionine (SAM). Each has a distinct aptamer domain that has evolved to specifically recognize a specific ligand. Currently, there are two distinct SAM riboswitches, one of which is dominant in gram positive bacteria, SAM-1, and one dominant in gram negative bacteria, SAM-II. The SAM-I riboswitch, which regulates methionine uptake and synthesis as well as SAM synthesis, contains a secondary structure comprised of four stem-loops surrounding a four-way junction motif. In the greater than approximately 300 SAM-I motifs that have been identified a number of nucleotides within and surrounding the junction are highly phylogenetically conserved (FIG. 1), including a consensus kink-turn motif and genetically-validated pseudoknot structure. Characterization of the binding of SAM analogs has indicated that this RNA recognizes every feature of the ligand, although the reactive methyl group indirectly. To further understand how this extreme degree of discrimination between SAM and closely related compounds can be achieved by this mRNA element, a crystal structure in complex with SAM has been solved.

Certain embodiments herein concern compositions and methods for selecting and identifying compounds that can activate, deactivate or block SAM-1 riboswitch. Activation or deactivation of a SAM-I riboswitch refers to the change in state of the riboswitch upon binding of the compound of interest, a test compound. The term trigger molecule is used herein to refer to molecules and compounds that can activate the SAM-I riboswitch.

Deactivation of a riboswitch refers to the change in state of the riboswitch when the trigger molecule is not bound. A riboswitch can be deactivated by binding of compounds other than the trigger molecule and in ways other than removal of the trigger molecule. Blocking of a riboswitch refers to a condition or state of the riboswitch where the presence of the trigger molecule does not activate the riboswitch.

In certain particular embodiments, methods of identifying a compound that interact with a SAM-I riboswitch include modeling the atomic structure of the SAM-I riboswitch with a test compound and determining if the test compound interacts with the SAM-I riboswitch. In accordance with these embodiments, the atomic contacts of the SAM-I riboswitch and test compound can be determined by means known in the art. Further, analogs of a compound known to interact with a SAM-I riboswitch can be generated by analyzing the atomic contacts for example the contacts that interact with ligand binding, then optimizing the atomic structure of the analog to maximize interaction. In certain embodiments, these methods can be used in a high throughput screen.

Other embodiments concern methods for identifying compounds that block a riboswitch. For example, an assay can be performed for assessing the induction or inhibition of SAM-I riboswitch in the presence of a test compound.

Some embodiments herein concern compositions and methods for identifying a test compound for significantly reducing the activity or inactivating a SAM-I riboswitch by binding the test compound to at least a portion of the atomic structure represented in FIG. 2A or 2B. In accordance with these embodiments, activity of the SAM-1 riboswitch can be measured by any methods known in the art. For example, the activity of the riboswitch can be measured in the presence or absence of a test compound in order to identify the efficiency of the test compound to reduce the activity of or inactivate the SAM-I riboswitch. Inactivation of a riboswitch in this manner can result from, for example, an alteration that prevents an S-adenosylmethionine molecule from binding; that prevents the change in state of the SAM-I riboswitch upon binding of S-adenosylmethionine; or the binding of the test compound interferes with ligand interaction or prevents the change in state of the SAM riboswitch.

In other embodiments, a test compound that activates a SAM-I riboswitch can be identified. For example, test compounds that activate a riboswitch can be identified by bringing into contact a test compound and a SAM-I riboswitch including at least a portion of the SAM-I riboswitch of FIG. 2A and FIG. 2B and assessing activation of the riboswitch. Activation of a SAM-I riboswitch can be assessed in any suitable manner. For example, activation of the SAM-I riboswitch can be measured by expression level of or modification of the expression level of a reporter gene in the presence or absence of the test compound. Examples of a reporter gene include, but are not limited to, beta-galactosidase, luciferase or green-fluorescence protein.

The SAM-I riboswitch is known to regulate multiple operons in a number of bacteria. Example bacteria contemplated herein include, but are not limited to, Staphylococcus spp., Bacillus spp., Listeria spp., Clostridia spp., Streptomyces spp., Thermoanaerobacteria spp. and a combination thereof.

Organization of Riboswitch RNAs

Structural probing studies demonstrate that bacterial riboswitch elements are composed of two domains: a natural aptamer that serves as the ligand-binding domain, and an ‘expression platform’ that interfaces with RNA elements that are involved in gene expression. Structural probing investigations suggest that the aptamer domain of most riboswitches adopts a particular secondary- and tertiary-structure fold when examined independently, that is essentially identical to the aptamer structure when examined in the context of the entire 5′ leader RNA. This implies that, in many cases, the aptamer domain is a modular unit that folds independently of the expression platform.

The ligand-bound or unbound status of the aptamer domain is interpreted through the expression platform, which is responsible for exerting an influence upon gene expression. The aptamer domains are highly conserved amongst various organisms, whereas the expression platform varies in sequence, structure, and in the mechanism by which expression of the appended open reading frame is controlled.

Aptamer domains for riboswitch RNAs typically range from ˜70 to 170 nt in length. Some aptamer domains, when isolated from the appended expression platform, exhibit improved affinity for the target ligand over that of the intact riboswitch. (˜10 to 100-fold). Presumably, there is an energetic cost in sampling the multiple distinct RNA conformations required by a fully intact riboswitch RNA, which is reflected by a loss in ligand affinity. Since the aptamer domain must serve as a molecular switch, this might also add to the functional demands on natural aptamers that might help rationalize their more sophisticated structures.

Riboswitch Regulation

Bacteria primarily use two methods for termination of transcription. Certain genes incorporate a termination signal that is dependent upon the Rho protein, while others make use of Rho-independent terminators (intrinsic terminators) to destabilize the transcription elongation complex. The latter RNA elements are composed of a GC-rich stem-loop followed by a stretch of 6-9 uridyl residues. Intrinsic terminators are widespread throughout bacterial genomes, and are typically located at the 3′-termini of genes or operons. Interestingly, an increasing number of examples are being observed for intrinsic terminators located within 5′-UTRs.

In certain examples, RNA polymerase responds to a termination signal within the 5′-UTR in a regulated fashion. Under certain conditions, the RNA polymerase complex is directed by external signals either to perceive or to ignore the termination signal. Although transcription initiation might occur without regulation, control over mRNA synthesis (and of gene expression) is ultimately dictated by regulation of the intrinsic terminator. Presumably, one of at least two mutually exclusive mRNA conformations results in the formation or disruption of the RNA structure that signals transcription termination. A trans-acting factor, which in some instances an RNA is generally required for receiving a particular intracellular signal and subsequently stabilizing one of the RNA conformations. Riboswitches offer a direct link between RNA structure modulation and the metabolite signals that are interpreted by the genetic control machinery.

Certain mRNAs involved in thiamine biosynthesis bind to thiamine (vitamin B₁) or its bioactive pyrophosphate derivative (TPP) without the participation of protein factors. The mRNA-effector complex adopts a distinct structure that sequesters the ribosome-binding site and leads to a reduction in gene expression. This metabolite-sensing mRNA system provides an example of a genetic “riboswitch” (referred to herein as a riboswitch) whose origin might predate the evolutionary emergence of proteins. It has been discovered that the mRNA leader sequence of the btuB gene of Escherichia coli can bind coenzyme B₁₂ selectively, and that this binding event brings about a structural change in the RNA that is important for genetic control. It was also discovered that mRNAs that encode thiamine biosynthetic proteins also employ a riboswitch mechanism.

Although certain specific natural riboswitches such as SAM-I riboswitch was one of the first examples of mRNA elements that control genetic expression by metabolite binding, it is suspected that this genetic control strategy may be widespread in biology. If these metabolites were being biosynthesized and used before the advent of proteins, then certain riboswitches might be modern examples of the most ancient form of genetic control. A search of genomic sequence databases has revealed that sequences corresponding to the TPP aptamer exist in organisms from bacteria, archaea and eukarya-largely without major alteration. Although new metabolite-binding mRNAs are likely to emerge as evolution progresses, it is possible that the known riboswitches are molecular fossils from the RNA world.

In certain embodiments, it is contemplated that a SAM-I Reporter system can be used to assess whether a test compound activates or inactivates the SAM-I riboswitch. In certain particular embodiments, an in vitro selection protocol can be designed for example to assess whether a test compound activates or deactivates the SAM-I riboswitch. In one particular embodiment, binding of the ligand can be monitored by a mobility-shift assay, known in the art, to discern free and bound RNA, providing a basis for selection of binding-competent RNAs. Ligand binding to the RNA can cause a conformational and/or secondary structural change in the RNA that can result in a change in its migration in a native polyacrylamide gel.

In certain embodiments, a detectable tag can be incorporated into the SAM-I riboswitch. In accordance with these embodiments, a test compound can be placed in contact with the SAM-I riboswitch and the interaction of the test compound and the SAM-I riboswitch assessed by measuring the presence or absence of a detectable tag. In certain particular examples, a detectable tag may be undetectable in the presence of a test compound thereby quenching the signal. This mechanism can be adapted to existing SAM-I riboswitches, as this method can take advantage of assessing a ligand-mediated interaction of the SAM-I riboswitch. In certain particular embodiments, a detectable tag can be placed within the ligand interaction region. In more particular embodiments, a detectable tag can be placed on any one of ligand binding nucleic acids, including but not limited to, A6, U6, G11, A45, C47, U57, G58, A86, U87, or a combination thereof of FIG. 2A or FIG. 2B of the SAM-I riboswitch. In these examples, a test compound can be combined with a SAM-I riboswitch depicted FIG. 2A or FIG. 2B and a detectable signal on the SAM-I riboswitch quenched when the test compound binds to at least one of the ligand-binding nucleic acids indicated above. In one particular example, a florescent tag molecule can be positioned in RNA adjacent to the binding site of SAM and binding can be monitored via a change in fluorescence of a reporter gene.

In other embodiments, control compounds can be used to assess interaction of the test compound compared to a known compound that interacts with a SAM-I riboswitch. To use riboswitches to report ligand binding by analyzing for a detectable tag, the appropriate construct can be determined empirically. The optimum length and composition of a test compound and its binding site on the riboswitch can be assessed systematically to result in the highest ligand binding region interaction possible. The validity of the assay can be determined by comparing apparent relative binding affinities of different SAM-I analogs, SAM-I antibodies or other SAM-I binding agents to a particular test compound (determined by the presence or level of detectable signal generation of the tag) to the binding constants determined by standard in-line probing.

In other embodiments, interaction of a test compound with at least a portion of the atomic structures depicted in FIG. 2A or FIG. 2B may be assessed by measuring uptake and/or synthesis of methionine and/or synthesis of SAM in a bacterial test cell system (e.g., cultures of B. subtilus). In accordance with these embodiments, a test compound confirmed to interact with at least a portion of the atomic structures depicted in FIG. 2A or 2B can be synthesized and/or purified for future use. In one example use, the test compound may be placed in contact with SAM-I riboswitch and the uptake and/or synthesis of methionine and/or synthesis of SAM can be measured. If a test compound is found to effectively block these functions, the test compound may be a candidate for use in inhibiting bacterial expansion or eliminating bacteria within a subject or a system.

In one example method, the structure depicted in FIG. 2A or 2B indicates that the RNA does not recognize the methyl group attached to the sulfur moiety, providing a place to build additional functionality that would be recognized by the RNA. Additionally, the positive charge on the sulfur is also recognized but not the sulfur atom itself, indicating that this region can be altered to ensure stability of the compound. Potential compounds could be computationally built and fit into the structure in place of SAM to determine if they would fit in the binding pocket of the riboswitch. Novel compounds can be synthesized by established chemistries and tested using a flourescence or footprinting type assay to ensure that they are recognized by the RNA.

It is contemplated herein that test compounds capable of associating with the atomic structures depicted in FIG. 2A or 2B may be a nucleic acid molecule, a small molecule, an antibody, a pharmaceutical agent, small peptide, peptide mimetic, nucleic acid mimetic, modified saccharide or aminoglycoside. Preferred test compound compositions would be small molecule mimetics of SAM or nucleic acid mimetics that build off of the adenosine moiety of SAM.

Kits

In still further embodiments, kits for methods and compositions described herein are contemplated. In one embodiment, the kits have a point-of care application, for example, the kits may have portability for use at a site of suspected bacterial outbreak. In another embodiment, a kit for treatment of a subject having a bacterial-induced infection is contemplated. In accordance with this embodiment, the kit may be used to reduce the bacterial infection of a subject.

The kits may include a container means. Any of the kits will generally include at least one vial, test tube, flask, bottle, syringe or other container means, into which the testing agent, may be preferably and/or suitably aliquoted. Kits herein may also include a means for comparing the results such as a suitable control sample such as a positive and/or negative control.

Nucleic Acids

In various embodiments, isolated nucleic acids may be used as test compounds for binding the atomic structure depicted in FIG. 2A or 2B. The isolated nucleic acid may be derived from genomic RNA or complementary DNA (cDNA). In other embodiments, isolated nucleic acids, such as chemically or enzymatically synthesized DNA, may be of use for capture probes, primers and/or labeled detection oligonucleotides.

A “nucleic acid” includes single-stranded and double-stranded molecules, as well as DNA, RNA, chemically modified nucleic acids and nucleic acid analogs. It is contemplated that a nucleic acid may be of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000 or greater nucleotide residues in length, up to a full length protein encoding or regulatory genetic element.

Construction of Nucleic Acids

Isolated nucleic acids may be made by any method known in the art, for example using standard recombinant methods, synthetic techniques, or combinations thereof. In some embodiments, the nucleic acids may be cloned, amplified, or otherwise constructed.

The nucleic acids may conveniently comprise sequences in addition to a portion of a SAM-I riboswitch. For example, a multi-cloning site comprising one or more endonuclease restriction sites may be added. A nucleic acid may be attached to a vector, adapter, or linker for cloning of a nucleic acid. Additional sequences may be added to such cloning and sequences to optimize their function, to aid in isolation of the nucleic acid, or to improve the introduction of the nucleic acid into a cell. Use of cloning vectors, expression vectors, adapters, and linkers is well known in the art.

Recombinant Methods for Constructing Nucleic Acids

Isolated nucleic acids may be obtained from bacterial or other sources using any number of cloning methodologies known in the art. In some embodiments, oligonucleotide probes which selectively hybridize, under stringent conditions, to the nucleic acids of a bacterial organism. Methods for construction of nucleic acid libraries are known and any such known methods may be used.

Nucleic Acid Screening and Isolation

Bacterial RNA or cDNA may be screened for the presence of an identified genetic element of interest using a probe based upon one or more sequences. Various degrees of stringency of hybridization may be employed in the assay. As the conditions for hybridization become more stringent, there must be a greater degree of complementarity between the probe and the target for duplex formation to occur. The degree of stringency may be controlled by temperature, ionic strength, pH and/or the presence of a partially denaturing solvent such as formamide. For example, the stringency of hybridization is conveniently varied by changing the concentration of formamide within the range up to and about 50%. The degree of complementarity (sequence identity) required for detectable binding will vary in accordance with the stringency of the hybridization medium and/or wash medium. In certain embodiments, the degree of complementarity can optimally be about 100 percent; but in other embodiments, sequence variations in the RNA may result in <100% complementarity, <90% complimentarily probes, <80% complimentarily probes, <70% complimentarily probes or lower depending upon the conditions. In certain examples, primers may be compensated for by reducing the stringency of the hybridization and/or wash medium.

High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. Other exemplary conditions are disclosed in the following Examples. It is understood that the temperature and ionic strength of a desired stringency are determined in part by the length of the particular nucleic acid(s), the length and nucleotide content of the target sequence(s), the charge composition of the nucleic acid(s), and by the presence or concentration of formamide, tetramethylammonium chloride or other solvent(s) in a hybridization mixture. Nucleic acids may be completely complementary to a target sequence or may exhibit one or more mismatches.

Nucleic Acid Amplification

Nucleic acids of interest may also be amplified using a variety of known amplification techniques. For instance, polymerase chain reaction (PCR) technology may be used to amplify target sequences directly from bacterial RNA or cDNA. PCR and other in vitro amplification methods may also be useful, for example, to clone nucleic acid sequences, to make nucleic acids to use as probes for detecting the presence of a target nucleic acid in samples, for nucleic acid sequencing, or for other purposes.

Synthetic Methods for Constructing Nucleic Acids

Isolated nucleic acids may be prepared by direct chemical synthesis by methods such as the phosphotriester method, or using an automated synthesizer. Chemical synthesis generally produces a single stranded oligonucleotide. This may be converted into double stranded DNA by hybridization with a complementary sequence or by polymerization with a DNA polymerase using the single strand as a template. While chemical synthesis of DNA is best employed for sequences of about 100 bases or less, longer sequences may be obtained by the ligation of shorter sequences.

Covalent Modification of Nucleic Acids

A variety of cross-linking agents, alkylating agents and radical generating species may be used to bind, label, detect, and/or cleave nucleic acids. In addition, covalent crosslinking to a target nucleotide using an alkylating agent complementary to the single-stranded target nucleotide sequence can be used. A photoactivated crosslinking to single-stranded oligonucleotides mediated by psoralen can be used. Use of N4,N4-ethanocytosine as an alkylating agent to crosslink to single-stranded oligonucleotides has also been disclosed. Various compounds to bind, detect, label, and/or cleave nucleic acids are known in the art.

Nucleic Acid Labeling

In various embodiments, tag nucleic acids may be labeled with one or more detectable labels to facilitate identification of a target nucleic acid sequence bound to a capture probe on the surface of a microchip. A number of different labels may be used, such as fluorophores, chromophores, radio-isotopes, enzymatic tags, antibodies, chemiluminescent, electroluminescent, affinity labels, etc. One of skill in the art will recognize that these and other label moieties not mentioned herein can be used. Examples of enzymatic tags include urease, alkaline phosphatase or peroxidase. Colorimetric indicator substrates can be employed with such enzymes to provide a detection means visible to the human eye or spectrophotometrically. A well-known example of a chemiluminescent label is the luciferin/luciferase combination.

In preferred embodiments, the label may be a fluorescent, phosphorescent or chemiluminescent label. Exemplary photodetectable labels may be selected from the group consisting of Alexa 350, Alexa 430, AMCA, aminoacridine, BODIPY 630/650, BODIPY 650/665, BODIPY-FL, BODIPY-R6G, BODIPY-TMR, BODIPY-TRX, 5-carboxy-4′,5′-dichloro-2′,7′-dimethoxy fluorescein, 5-carboxy-2′,4′,5′,7′-tetrachlorofluorescein, 5-carboxyfluorescein, 5-carboxyrhodamine, 6-carboxyrhodamine, 6-carboxytetramethyl amino, Cascade Blue, Cy2, Cy3, Cy5,6-FAM, dansyl chloride, Fluorescein, HEX, 6-JOE, NBD (7-nitrobenz-2-oxa-1,3-diazole), Oregon Green 488, Oregon Green 500, Oregon Green 514, Pacific Blue, phthalic acid, terephthalic acid, isophthalic acid, cresyl fast violet, cresyl blue violet, brilliant cresyl blue, para-aminobenzoic acid, erythrosine, phthalocyanines, azomethines, cyanines, xanthines, succinylfluoresceins, rare earth metal cryptates, europium trisbipyridine diamine, a europium cryptate or chelate, diamine, dicyanins, La Jolla blue dye, allopycocyanin, allococyanin B, phycocyanin C, phycocyanin R, thiamine, phycoerythrocyanin, phycoerythrin R, REG, Rhodamine Green, rhodamine isothiocyanate, Rhodamine Red, ROX, TAMRA, TET, TRIT (tetramethyl rhodamine isothiol), Tetramethylrhodamine, and Texas Red. These and other labels are available from commercial sources, such as Molecular Probes (Eugene, Oreg.).

Solid Supports

Solid supports are solid-state substrates or supports with which molecules (such as trigger molecules, e.g., SAM) and riboswitches (or other components used in, or produced by, the disclosed methods) can be associated. Riboswitches and other molecules can be associated with solid supports directly or indirectly. For example, analytes (e.g., trigger molecules, test compounds) can be bound to the surface of a solid support or associated with capture agents (e.g., compounds or molecules that bind an analyte) immobilized on solid supports. As another example, riboswitches can be bound to the surface of a solid support or associated with probes immobilized on solid supports. An array is a solid support to which multiple riboswitches, probes or other molecules have been associated in an array, grid, or other organized pattern.

In some embodiments, a solid-state substrate may be used. Solid supports contemplated of use can include any solid material with which components can be associated, directly or indirectly. These material include but are not limited to acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multi-well glass slide can be employed.

In certain embodiments, an array can include a plurality of riboswitches, trigger molecules, other molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of riboswitches, trigger molecules, other molecules, compounds and/or probes can be distributed over any number of solid supports. For example, in some embodiments, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, riboswitches, trigger molecules, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances. In accordance with these examples, components can be associated or immobilized on a solid support at any density. Components can be immobilized to the solid support at a density exceeding 400 different components per cubic centimeter. Arrays of components can have any number of components depending on the circumstances.

Pharmaceutical Compositions

In certain embodiments, compositions of identified test compounds may be generated for use in a subject having a bacterial infection in order to reduce or eliminate the infection in the subject. In accordance with these embodiments, the compositions can be administered in a subject in a biologically compatible form suitable for pharmaceutical administration in vivo. By “biologically compatible form suitable for administration in vivo” is meant a form of the active agent (e.g., pharmaceutical chemical, protein, gene, antibody etc of the embodiments) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically effective amount of an antibody or nucleic acid molecule reactive with at least a portion of SAM-I riboswitch depicted in FIG. 2A or FIG. 2B may vary according to factors such as the disease state, age, sex, and weight of the individual, and the ability of antibody to elicit a desired response in the individual. Dosage regima may be adjusted to provide the optimum therapeutic response. For example, several divided doses may be administered daily or the dose may be proportionally reduced as indicated by the exigencies of the therapeutic situation.

In one embodiment, the compound (e.g., pharmaceutical chemical, nucleic acid molecule, gene, protein, antibody etc of the embodiments) may be administered in a convenient manner such as by injection such as subcutaneous, intravenous, by oral administration, inhalation, transdermal application, intravaginal application, topical application, intranasal or rectal administration. Depending on the route of administration, the active compound may be coated in a material to protect the compound from the degradation by enzymes, acids and other natural conditions that may inactivate the compound. In a preferred embodiment, the compound may be orally administered. In another preferred embodiment, the compound may be inhaled in order to make the compound bioavailable to the lung.

A compound may be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. To administer a compound that stimulates or inhibits a SAM-I riboswitch by other than parenteral administration, it may be necessary to coat the compound with, or co-administer the compound with, a material to prevent its inactivation. Enzyme inhibitors include pancreatic trypsin inhibitor, diisopropylfluorophosphate (DEP) and trasylol. Liposomes include water-in-oil-in-water emulsions as well as conventional liposomes. The active agent may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyetheylene glycol, and the like), and suitable mixtures thereof The proper fluidity can be maintained, for example, by the use of a coating such as lecithin, by the maintenance of the required particle size in the case of dispersion and by the use of surfactants. Prevention of microorganisms can be achieved by various antibacterial and antifungal agents (i.e., parabens, chlorobutanol, phenol, ascorbic acid, thimerosal, and the like). In many cases, it will be preferable to include isotonic agents, for example, sugars, polyalcohols such as manitol, sorbitol, sodium chloride in the composition. A compound such as aluminum monostearate and gelatin can be included to prolong absorption of the injectable compositions.

Sterile injectable solutions can be prepared by incorporating active compound (e.g., a chemical that modulates the SAM-I riboswitch) in the required amount in an appropriate solvent with one or a combination of ingredients enumerated above, as required, followed by filtered sterilization. Generally, dispersions are prepared by incorporating the active compound into a sterile vehicle that contains a dispersion medium and other required ingredients from those enumerated above. In the case of sterile powders for the preparation of sterile injectable solutions, the preferred methods of preparation are vacuum drying and freeze-drying which yields a powder of the active ingredient (i.e., a chemical agent, antibody etc.) plus any additional desired ingredient from a previously sterile-filtered solution thereof

When the active agent is suitably protected, as described above, the composition may be orally administered (or otherwise indicated), for example, with an inert diluent or an assimilable edible carrier. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active agent and the particular therapeutic effect to be achieved, and (b) the limitations inherent an active agent for the therapeutic treatment of individuals.

### EXAMPLES

The following examples are included to illustrate various embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that many changes may be made in the specific embodiments which are disclosed and still obtain a like or similar result without departing from the spirit and scope of the invention.

RNA preparation. A 94 nucleotide construct consisting of the sequence for the SAM riboswitch from the metF-metH2 operon of T. tencongensis was constructed by PCR using overlapping DNA oligonucleotides (e.g., Integrated DNA Technologies). The resulting fragment contained sites for the restriction enzymes EcoRI and NgoMIV and was ligated into plasmid vector pRAV12, which is designed for either native or denaturing purification of RNA. The cloned sequence was verified by sequencing. Transcription template was prepared by PCR using primers directed against the T7 promoter (SEQ ID NO:1: 5′, GCGCGCGAATTCTAATAC GACTCACTATAG, 3′) and the HδV ribozyme in the vector

The global architecture of the SAM-I riboswitch aptamer domain is established through two coaxial stacks of helices. The first comprises the P1/P4 stack (FIGS. 2A and 2B) in which the J4/1 strand containing three highly conserved adenosine residues are stacked in between the two helices with no disruption to the A-form geometry between them. One of the adenosines in the J4/1 region interacts with U64 of J3/4 and A24 of L2 to form a triple that serves to tie the P1/P4 stack against P2b. The other set of stacked helices is P2a/P3 (FIG. 1), the binding pocket for the S-adenosylmethionine (SAM) ligand lies in a region where the P1 and P3 helices come into close contact. This arrangement of the two domains (P1/P4 and P2/P3) is topologically similar to the arrangement of the P4P6 and P3P7 that form the catalytic core in group I introns. Despite very different secondary structures, the similarity of interdomain arrangements suggests that this is a very favorable topology for forming ligand binding and catalytic sites in RNAs.

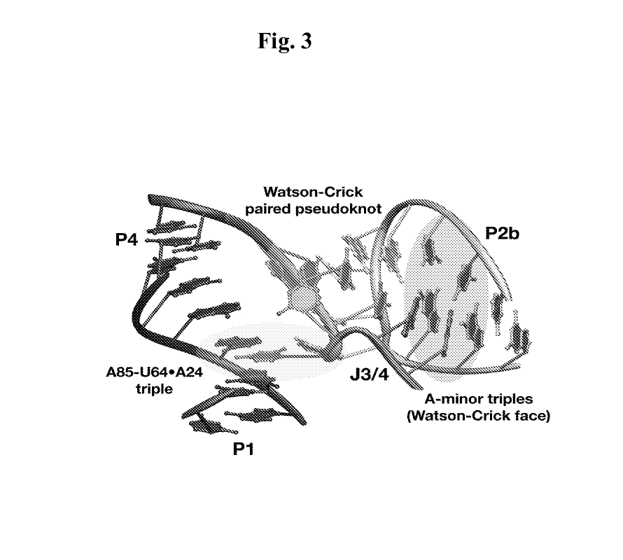

The global tertiary architecture of the riboswitch is believed to be established through a series of interactions between L2, J3/4 and J4/1 (See FIG. 3). Within P2 is a kink-turn motif that creates a ˜100° bend in the helix; this structure of this motif is identical to that observed in the ribosome the Box C/D snoRNP and the U4 snRNP. The kink-turn in the SAM-I riboswitch conforms to the consensus motif and has a structure that is substantially identical to that observed in other RNAs. This motif allows for P2b to be oriented back towards the P1/P4 stack allowing for a pseudoknot interaction between L2 and J3/4, which was predicted from phylogeny and genetic experiments. This pseudoknot is tied against P1/P4 through an (A85-U64)•A24 triple. J3/4 is further tied to P2b through two adenine-mediated triples in which the Watson-Crick face of A61 and A62 interact with the minor groove of the G22-C30 and G23-C29 pairs, respectively. This form of adenosine triple is not nearly as common as the A-minor triple motif but has been observed in the 16S rRNA and in RNA-mediated crystal contacts. Without being bound by any theory, it is believed that these elements of tertiary architecture are formed prior to ligand binding; in-line probing of several different SAM-I riboswitches have all demonstrated that nucleotides involved in formation of the pseudoknot and the adenine-triples are protected from cleavage. This indicates that these residues are not significantly conformationally flexible in the free state. Thus, the SAM-I riboswitch, like the guanine riboswitch, has a pre-established global architecture that organizes the RNA for ligand recognition.

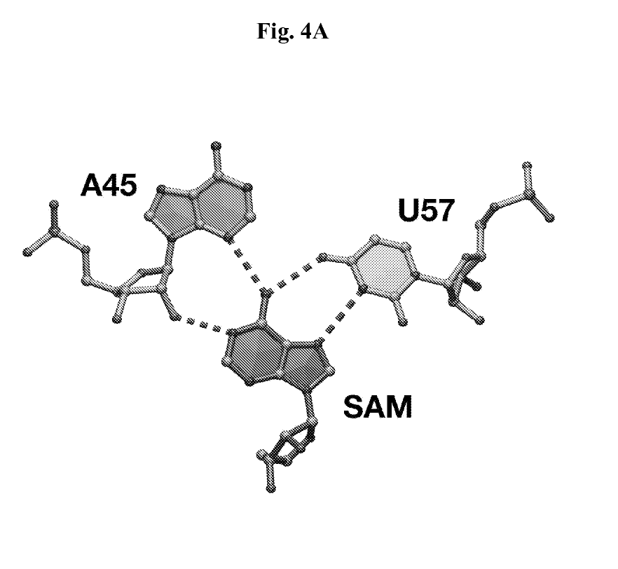

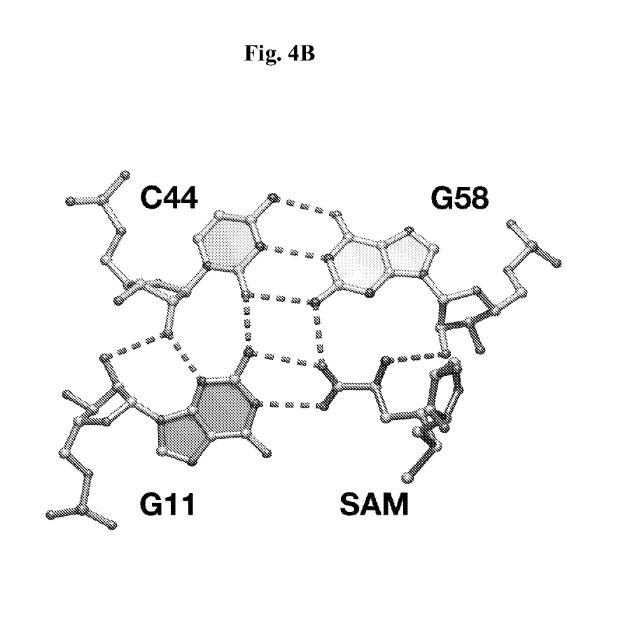

S-adenosylmethionine is specifically recognized by the riboswitch within a pocket created between the P1 and P3 helices (FIGS. 2A and 2B). The ligand adopts a conformation in which the methionine moiety stacks upon the adenine ring, such that the main chain atoms of the amino acid are spatially adjacent to the Watson-Crick face of the adenine base. The adenine ring is the central base of a base triple between A45 and U57 (FIG. 4A). These two nucleotides are part of an asymmetric internal loop motif (5′AA/U) in helix 3 (FIG. 1) that is believed to be universally conserved among SAM-I riboswitch RNAs. Interestingly, it is believed that the adenine moiety needs to disrupt a Watson-Crick A-U pair in the free form of the riboswitch RNA in order to establish the base triple. The placement of the adenine ring is further stabilized by stacking with C47, which is part of a dinucleotide platform adjacent the P3 internal loop. The main chain atoms of the methionine moiety are extensively recognized through a series of hydrogen bonds with the C44-G58 base pair of the P3 helix and G11 of J1/2 region to form a pseudo-quadruple (FIG. 4B). This arrangement is consistent with the observation that while S-adenosylhomocysteine (SAH) is tightly bound by the RNA (400 nM in the yitJ homolog from B. subtilis), the analog S-adenosylcysteine (SAC), which contains one less side chain methylene group, is bound very weakly (˜30 μM). Shortening of the side chain would prevent the main chain carboxylate group from being able to effectively hydrogen bond with G11.

Many riboswitches and aptamers recognize ligands with negatively charged groups including ATP thiamine pyrophosphate, flavin mononucleotide, as well as SAM. Negative charge is expected to be difficult for the polyanionic RNA to recognize; aptamers selected to bind SAM indeed bind the adenine and ribose moiety well, but do not recognize the methionine functional group. In this structure, it is clear the negatively charged functional group is recognized by the Watson-Crick face of a guanine residue (G11). This is very analogous to an acetate ion binding in the purine riboswitch, as well as binding of non-bridging phosphate oxygens in the backbone of the GAAA tetraloop, SRP RNA, and the ribosomal RNA. It is likely that a very general mode for anion recognition, particularly carboxy and phosphate groups, is through the N1 and N2 groups of unpaired guanine residues.

The other half of the binding pocket for SAM is created by the minor groove of the P1 helix, adjacent to the universally conserved A6-U88 and U7-A87 base pairs. The ribose sugar of SAM bridges the P1 and P3 helices via interactions between SAM-2′-OH and O4′ of C47 in P3, SAM-3′-OH and O4′ of U7, and SAM-O4′ and O2′ of U88. The sulfur atom is situated approximately 4 A from the O2 carbonyl oxygens of U7 and U88 (data not shown). This positioning likely serves as the basis for a 100-fold preference for SAM over SAH. In SAM, the positively charged sulfur would be positioned to make favorable electrostatic interactions with the carbonyls of the minor groove of P1. This electrostatic interaction is consistent with observations that the identity of the charged moiety at this position is not important, but the presence of a formal positive charge or high partial positive charge is sensed. While in the electron density maps we did not observe a region of clear electron density around the sulfur atom that would correspond to the methyl group of SAM, its position can be readily inferred as the sulfur is biologically always found in the S configuration. The model of SAM places the methyl group facing towards a solvent cavity within the interior of the folded RNA. This is consistent with the biochemical observations that have suggested that the methyl group is not directly recognized by the RNA.

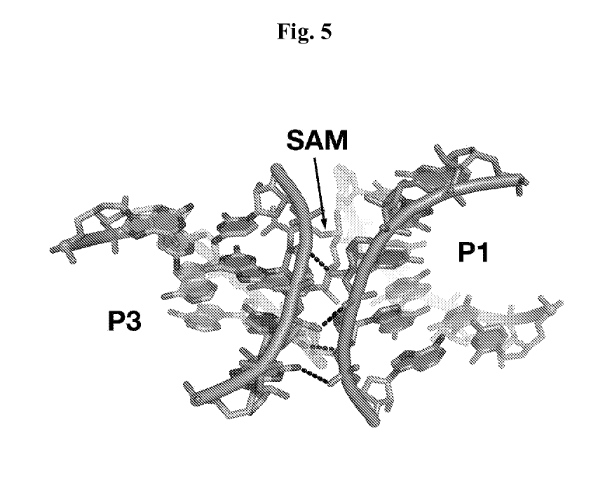

The binding site for SAM can be created through the docking of the minor groove faces of the P1 and P3 helices. While SAM has a fairly loose association with the P1 helix, as suggested by the long hydrogen-bonding distances between SAM and functional groups of P1, the backbone of P1 makes intimate contacts with the minor groove of P3. These interactions involve a mixture of hydrogen bonding and van der Waals contacts between the backbone ribose/phosphate atoms of U88-A90 in helix P1 and C47, C48, G56, U57 and G58 of helix P3 (FIG. 5). Like the purine riboswitch, the P1 helix is stabilized via a series of tertiary interactions that form only upon association of ligand. This suggests a common mechanism for how riboswitches are able to transduce a ligand binding event into changes in gene expression. All known riboswitches that regulate at the transcriptional level, which is the majority of those characterized, have the equivalent of a P1 helix involving the pairing of the 5′- and 3′-ends of the aptamer domain. The 3′-side of the P1 helix is an integral part of a structural switch involving two mutually exclusive secondary structures (FIG. 1). Ligand binding to the aptamer domain induces the formation of a set of tertiary interactions with the P1 helix that certainly serve to stabilize it and favor its formation over the alternate structure. In the case of the guanine riboswitch, this involves formation of base triple interactions, while in the SAM-I riboswitch backbone-minor groove interactions occur between P1 and P3. While the secondary structure of the aptamer domain and the nature of its cognate ligand differs significantly in each class of riboswitch, this study suggests that there exists strong similarities in how they achieve efficient gene regulation.

Additional objects, advantages, and novel features of this invention will become apparent to those skilled in the art upon examination of the following examples thereof, which are not intended to be limiting.

Methods and Materials.

RNA preparation. In one exemplary method, a 94 nucleotide construct consisting of the sequence for the SAM riboswitch from the metF-metH2 operon of T. tencongensis was constructed by PCR using overlapping DNA oligonucleotides (Integrated DNA Technologies). The resulting fragment contained site for the restriction enzymes EcoRI and NgoMIV and was ligated into plasmid vector pRAV12, which is designed for either native or denaturing purification of RNA. The cloned sequence was verified by sequencing. Transcription template was prepared by PCR using primers directed against the T7 promoter (SEQ ID NO:1 5′, GCGCGCGAATTCTAATACGACTCACTATAG) and the HδV ribozyme in the vector. Because the HδV sequence in the vector is mutated to be active only in the presence of imidazole, the primer used contained the single-base correction required for wild-type activity. RNA was transcribed in a 12.5 mL reaction containing 30 mM Tris-HCl (pH 8.0), 10 mM DTT, 0.1% Triton X-100, 0.1 mM spermidine-HCl, 4 mM each NTP, 24 mM MgCl₂, 0.25 mg/mL T7 RNA polymerase, 1 mL of 0.5 μM template, and 0.32 unit/mL inorganic pyrophosphatase to suppress formation of insoluble magnesium pyrophosphate. The transcription reaction was allowed to proceed for two hours at 37° C., supplemented with an addition 20 mM MgCl₂ and incubated at 60° C. for 15 minutes to enhance cleavage of the HδV ribozyme at the 3′ end of the riboswitch construct. RNA was then ethanol precipitated at −20° C. overnight, purified by denaturing PAGE (12% polyacrylamide, 1× TBE, 8 M urea). The band of interest was visualized by UV shadowing, excised, and electroeluted overnight in 1× TBE to extract RNA from the gel. The eluted fraction was exchanged three times into 10 mM Na-MES at pH 6.0 using a 10,000 MWCO centrifugal filter, then refolded by heating to 95° C. for three minutes followed by snap cooling. The refolded RNA was exchanged once into 10 mM Na-MES pH 6.0, 2 mM MgCl₂. The final yield was ˜500 μL of RNA at a concentration of 400 μM as judged by absorbance at 260 nm and the calculated extinction coefficient. RNA was stored at −20° C.

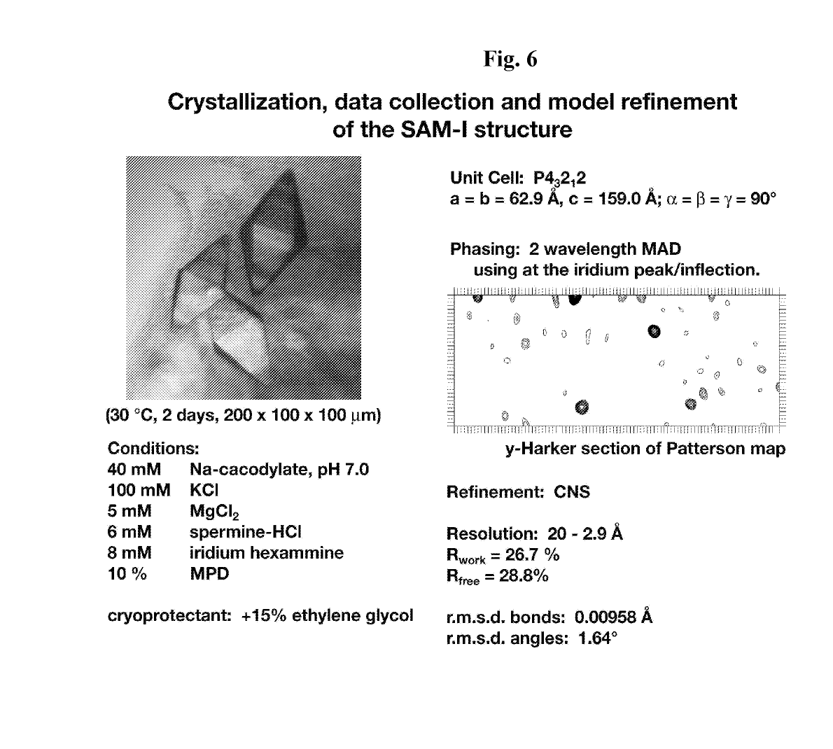

Crystallization. In another example, SAM was added to RNA stock right before the RNA was set-up for crystallization by directly pipetting a predetermined amount of 100 mM SAM stock into the RNA solution. Final concentration of SAM in the RNA was approximately 5 mM. Bound RNA was crystallized by the hanging drop vapor diffusion method. RNA was mixed 1:1 with a solution consisting of 8 mM iridium hexammine, 100 mM KCl, 5 mM MgCl₂, 10% MPD, 0 mM Na-cacodylate pH 7.0, and 6 mM spermine HCl. The drop was seeded with seed-stock grown in 27 mM spermine, 34 mM Na-cacodylate, 17 mM BaCl₂, 8.5% MPD, and 34 mM KCl. Crystals grew in a diamond morphology to their maximum size (˜0.3 mm on the edge) in 48 hours at 30° C. and were cryoprotected by soaking the crystals for at least 5 minutes in 50 mL of a solution consisting of the motherliquor plus 15% ethylene glycol. Crystals were then flash-frozen in liquid nitrogen. Data was collected on beamline 8.2.1 at the Advanced Light Source in Berkeley, Calif. using an inverse beam experiment at two wavelengths. Data was indexed, integrated, and scaled using D*TREK. The crystals belong to the P4₃2₁2 space group (a=62.90 Å, b=62.90 Å, c=158.97 Å, α=β=γ=90°) and have one molecule per asymmetric unit. All the data used in this example phasing and refining came from one crystal (see FIG. 6 and Table 3).

Preparation of iridium hexaammine.

The iridium hexaammine was prepared according to methods outlined in the literature. Two grams iridium chloride (IrCl₃) (Aldrich) and 35 mL ammonium hydroxide were added to a heavy-walled ACE pressure tube (Aldrich). The tube was then sealed and incubated in a 150° C. silicone oil bath for four days. The reaction was then allowed to completely cool and incubated on slushy ice. The clear, light brown solution was then filtered and evaporated to dryness under vacuum. While evaporating, the solution was heated to 50° C. using a waterbath. The resulting solid was then resuspended in 5 mL of water and transferred to a 50 mL conical tube. Two mL of concentrated HCl was then added to the solution. Precipitate was spun down in a centrifuge and the light yellow supernatant was discarded. Pellet was washed three times with 10 mL of a 2:1 (v/v) water:conc. HCl solution by vigorous vortexing followed by centrifugation. Supernatant was discarded after each wash. Pellet was then washed three times in absolute ethanol, air-dried and resuspended in ˜3 mL ddH₂O. Solution was centrifuged one more time to remove insoluble material. The resulting supernatant should show a clear absorbance maxima at 251 nm and concentration can be calculated using the extinction coefficient 92 M⁻¹cm⁻¹ at 251 nm. Typical yield is 50%. Supernatant was then aliquoted into fresh Eppendorf tubes and stored at −20° C.

Phasing and structure determination. Phases were determined by multi-wavelength anomalous diffraction (MAD) using data that extended to 2.8 Å. The peak and inflection wavelength datasets were merged and scaled in CNS and Patterson maps were then calculated for both space groups P4₁2₁2 and P4₃2₁2. From the maps it was determined that there were four possible iridium sites within the unit cell, although most if not all had less than full occupancy. A CNS heavy-atom search for four possible sites was then carried out in both space groups, and both space groups yielded 94 possible solutions. The best of these were used to calculate predicted Patterson maps, which showed peaks that correlated very well with those seen in the original maps in all four Harker sections. The best solution sites were used to calculate phases in CNS. The resulting density map for P4₁2₁2 was uninterpretable, whereas the map for P4₃2₁2 clearly showed features that were macromolecular, such as RNA helix backbones and base-stacking. The phasing solution found by CNS had a figure of merit of 0.6332 which was further improved to 0.8846 following a round of density modification with the solvent level set to 0.46. The phasing power at the peak wavelength was 3.3 with a R_{cullis} of 0.39 (acentric).

Using methods known in the art, the model was built in O and refined in CNS in iterative rounds. The RNA nucleotides were placed in the first round, the iridium hexaammines were placed in the second round, and then in the third round two magnesium ions were placed based on their position in the density with respect to the sugar-phosphate backbone of the RNA. Once the ions were in place the SAM was built. Structure, parameter, and topology files for iridium hexaammine and SAM were downloaded from HIC-Up (Hetero-compound Information Centre-Uppsala); the parameters for Mg²⁺ ions were already loaded into CNS. The compact conformation of the SAM molecule was chosen to fit the density seen in the binding pocket, and in order to get the model molecule to fit the density the energy parameters in the SAM parameter file downloaded from HIC-Up had to be changed. This was followed by one round of water-picking carried out by CNS. Waters were chosen based on peak size in an anomalous difference map. The minimum was set to 2.5σ with the additional parameters that the B-factor could be no greater than 200, and the peak must be within hydrogen bonding distance of the oxygens and nitrogens in the RNA. Each round of model-building was followed by a simulated annealing run and B-factor refinement using CNS. R_{free} was monitored in each round to ensure that it was dropping. Sugar puckers were restrained in most cases to C3′ endo, except for residues A9, A14, A33, A51, U63, and G74 which were restrained to C2′ endo. Some of the figures were prepared using Ribbons 3.0 and Pymol.

TABLE 1
Data collection, phasing and refinement statistics
Iridium hexamine
Data collection

| Space group | P4₃2₁2 |
|---|---|
| Cell dimensions | |
| a, b, c (Å) | 62.90, 62.90, 158.97 |
| α, β, γ (°) | 90, 90, 90 |

| | Peak | Inflection |
|---|---|---|
| Wavelength | 1.10532 Å | 1.10573 Å |
| Resolution^{b} (Å) | 50-2.8 (2.9-2.8) | 50-2.8 (2.9-2.8) |
| R_{merge} | 0.072 (0.426) | 0.073 (0.446) |
| I/δI | | 18.2 (4.3) |
| Completeness (%) | 99.6 (99.9) | 99.4 (100) |
| Redundancy | 14.64 (11.74) | 14.68 (11.92) |

Refinement

| | |
|---|---|
| Resolution^{b} (Å) | 50-2.9 (3.0-2.9) |
| No. reflections | 13415 (99.3%) |
| R_{work}/R_{free} | 0.2730 (0.4662)/0.2830 (0.4286) |
| No. atoms | 2174 |
| RNA | 2029 |
| Ligand/Ions | 27/30 |
| Water | 88 |
| B-factors | 70.2578 |
| RNA | 70.1875 |
| Ligand/Ions | 59.9444/79.5450 |
| Water | 80.6735 |
| R.m.s deviations | |
| Bond lengths (Å) | 0.006976 |
| Bond angles (°) | 1.37319 |

* Data collected on one crystal.
*Highest resolution shell is shown in parenthesis.

TABLE 2
Nucleotides important for formation of tertiary interactions
(global architecture) and ligand recognition.

| | Nucleotide* | Contact | Conservation^{‡} |
|---|---|---|---|
| Tertiary contacts | G11 | C44 | >97%, >97% |
| | A12 | G43-C59 pair | >97%, >90% |
| | G13 | C41 (Watson-Crick) | >75%, >75% |
| | A24 | A85 | >90%, >75% |
| | C25 | G68 (Watson-Crick) | >75%, >75% |
| | U26 | U67 | Not cons. |
| | G27 | C66 | >90%, >90% |
| | G28 | C65 | >90%, >90% |
| | A46 | C47 | >90%, >97% |
| | A61 | G22-C30 pair | >90%, >75% |
| | A62 | G23-C29 pair | >75%, >97% |
| | U64 | A85 | >75%, >75% |
| | U88 | G58 | >97%, >97% |
| | G89 | U57, C47 | >97% all |
| | A90 | G56 | >75%, >97% |

| | RNA nt. | SAM moiety | Conservation |
|---|---|---|---|
| Ligand recognition | A6 | Ribose sugar | >97% |
| | U6 | Ribose sugar, sulfur | >97% |
| | G11 | Methionine side chain | >97% |
| | A45 | Adenine moiety | >97% |
| | C47 | Adenine moiety | >97% |
| | U57 | Adenine moiety | >97% |
| | G58 | Methionine side chain | >97% |
| | A86 | Ribose sugar | >97% |
| | U87 | Ribose sugar | >97% |

*Nucleotide numbering consistent with that used in the RNA crystallized.
^{‡}Conservation based upon alignment of ˜100 SAM-I sequences (Rfam database, http://www.sanger.ac.uk/Software/Rfam/)

TABLE 3
REMARK coordinates from simulated annealing refinement
REMARK refinement resolution: 50-2.9 A

| | |
|---|---|
| REMARK starting | r= 0.2651 free_r= 0.2890 |
| REMARK final | r= 0.2667 free_r= 0.2879 |

REMARK rmsd bonds= 0.009579 rmsd angles= 1.64239
REMARK wa= 4
REMARK target= mlhl md-method= torsion annealing schedule= slowcool
REMARK starting temperature= 4000 total md steps= 80 * 6
REMARK sg= P4(3)2(1)2 a= 62.901 b= 62.901 c= 158.967 alpha= 90 beta= 90
gamma= 90
REMARK parameter file 1 : CNS_TOPPAR: dna-rna_rep.param
REMARK parameter file 2 : sam3.param
REMARK parameter file 3 : CNS_TOPPAR: water_rep.param
REMARK parameter file 5 : ion2.param
REMARK molecular structure file: samrnaZ.mtf
REMARK input coordinates: minimizeZ_B2.pdb
REMARK anomalous f′ f″ library: fp_fdp_groupA6.lib
REMARK reflection file= scaleMAD.cv
REMARK reflection file= mad_phaseIRMADBP43212.hkl
REMARK additional restraints file: dna-rna_restraintsE.def
REMARK ncs= none
REMARK B-correction resolution: 6.0-2.9
REMARK initial B-factor correction applied to f_w1:
REMARK B11= −11.957 B22= −11.957 B33= 23.914
REMARK B12= 0.000 B13= 0.000 B23= 0.000
REMARK B-factor correction applied to coordinate array B: −0.634
REMARK bulk solvent: density level= 0.8529 e/A\hat{ }3, B-factor= 300 A\hat{ }2
REMARK reflections with |Fobs|/sigma_F <0.0 rejected
REMARK reflections with |Fobs| >10000 * rms(Fobs) rejected
REMARK anomalous diffraction data was input

| | | |
|---|---|---|
| REMARK theoretical total number of refl. in resol. range: | 13511 | (100.0%) |
| REMARK number of unobserved reflections (no entry or \|F\|=0): | 96 | ( 0.7%) |
| REMARK number of reflections rejected: | 0 | ( 0.0%) |
| REMARK total number of reflections used: | 13415 | ( 99.3%) |
| REMARK number of reflections in working set: | 12416 | ( 91.9%) |
| REMARK number of reflections in test set: | 999 | ( 7.4%) |

CRYST1 62.901 62.901 158.967 90.00 90.00 90.00 P 43 21 2
REMARK FILENAME=“annealZ_B4_1.pdb”
REMARK DATE: 8-Feb-06 04:28:37 created by user: montangr
REMARK VERSION: 1.1

| | | | | | | | | | | |
|---|---|---|---|---|---|---|---|---|---|---|
| ATOM | 1 | O5T | GUA | 1 | 66.683 | 54.256 | 31.032 | 1.00 | 83.68 | |
| ATOM | 2 | P | GUA | 1 | 66.835 | 54.687 | 32.502 | 1.00 | 83.36 | |
| ATOM | 3 | O1P | GUA | 1 | 67.821 | 55.847 | 32.631 | 1.00 | 83.57 | |
| ATOM | 4 | O2P | GUA | 1 | 65.490 | 54.992 | 33.172 | 1.00 | 81.66 | |
| ATOM | 5 | O5′ | GUA | 1 | 67.506 | 53.445 | 33.341 | 1.00 | 79.16 | |
| ATOM | 6 | C5′ | GUA | 1 | 68.808 | 52.929 | 32.997 | 1.00 | 72.71 | |
| ATOM | 7 | C4′ | GUA | 1 | 69.342 | 52.033 | 34.100 | 1.00 | 69.78 | |
| ATOM | 8 | O4′ | GUA | 1 | 69.939 | 52.838 | 35.158 | 1.00 | 66.22 | |
| ATOM | 9 | C1′ | GUA | 1 | 69.690 | 52.242 | 36.422 | 1.00 | 63.61 | |
| ATOM | 10 | N9 | GUA | 1 | 68.769 | 53.113 | 37.142 | 1.00 | 59.52 | |
| ATOM | 11 | C4 | GUA | 1 | 68.410 | 53.068 | 38.479 | 1.00 | 56.49 | |
| ATOM | 12 | N3 | GUA | 1 | 68.874 | 52.213 | 39.404 | 1.00 | 55.16 | |
| ATOM | 13 | C2 | GUA | 1 | 68.286 | 52.395 | 40.583 | 1.00 | 54.45 | |
| ATOM | 14 | N2 | GUA | 1 | 68.607 | 51.617 | 41.626 | 1.00 | 54.23 | |
| ATOM | 15 | N1 | GUA | 1 | 67.338 | 53.345 | 40.827 | 1.00 | 52.87 | |
| ATOM | 16 | C6 | GUA | 1 | 66.852 | 54.236 | 39.883 | 1.00 | 54.26 | |
| ATOM | 17 | O6 | GUA | 1 | 65.983 | 55.064 | 40.201 | 1.00 | 55.11 | |
| ATOM | 18 | C5 | GUA | 1 | 67.458 | 54.048 | 38.629 | 1.00 | 55.33 | |
| ATOM | 19 | N7 | GUA | 1 | 67.246 | 54.714 | 37.435 | 1.00 | 57.56 | |
| ATOM | 20 | C8 | GUA | 1 | 68.047 | 54.131 | 36.587 | 1.00 | 58.84 | |
| ATOM | 21 | C2′ | GUA | 1 | 69.048 | 50.883 | 36.141 | 1.00 | 66.60 | |
| ATOM | 22 | O2′ | GUA | 1 | 70.070 | 49.922 | 35.982 | 1.00 | 66.97 | |
| ATOM | 23 | C3′ | GUA | 1 | 68.334 | 51.149 | 34.821 | 1.00 | 68.51 | |
| ATOM | 24 | O3′ | GUA | 1 | 68.102 | 49.938 | 34.106 | 1.00 | 68.62 | |
| ATOM | 25 | P | GUA | 2 | 66.687 | 49.177 | 34.246 | 1.00 | 68.94 | |
| ATOM | 26 | O1P | GUA | 2 | 65.559 | 50.138 | 34.075 | 1.00 | 68.40 | |
| ATOM | 27 | O2P | GUA | 2 | 66.770 | 47.973 | 33.393 | 1.00 | 69.33 | |
| ATOM | 28 | O5′ | GUA | 2 | 66.653 | 48.669 | 35.751 | 1.00 | 67.96 | |
| ATOM | 29 | C5′ | GUA | 2 | 67.438 | 47.559 | 36.151 | 1.00 | 64.12 | |
| ATOM | 30 | C4′ | GUA | 2 | 67.352 | 47.376 | 37.641 | 1.00 | 61.63 | |
| ATOM | 31 | O4′ | GUA | 2 | 67.613 | 48.663 | 38.266 | 1.00 | 58.76 | |
| ATOM | 32 | C1′ | GUA | 2 | 66.860 | 48.777 | 39.450 | 1.00 | 55.68 | |
| ATOM | 33 | N9 | GUA | 2 | 66.027 | 49.953 | 39.350 | 1.00 | 51.79 | |
| ATOM | 34 | C4 | GUA | 2 | 65.271 | 50.512 | 40.361 | 1.00 | 51.24 | |
| ATOM | 35 | N3 | GUA | 2 | 65.152 | 50.043 | 41.624 | 1.00 | 50.23 | |
| ATOM | 36 | C2 | GUA | 2 | 64.362 | 50.803 | 42.352 | 1.00 | 49.82 | |
| ATOM | 37 | N2 | GUA | 2 | 64.105 | 50.482 | 43.616 | 1.00 | 50.74 | |
| ATOM | 38 | N1 | GUA | 2 | 63.753 | 51.935 | 41.896 | 1.00 | 49.79 | |
| ATOM | 39 | C6 | GUA | 2 | 63.858 | 52.430 | 40.608 | 1.00 | 50.39 | |
| ATOM | 40 | O6 | GUA | 2 | 63.254 | 53.465 | 40.297 | 1.00 | 52.03 | |
| ATOM | 41 | C5 | GUA | 2 | 64.692 | 51.623 | 39.808 | 1.00 | 49.54 | |
| ATOM | 42 | N7 | GUA | 2 | 65.063 | 51.758 | 38.484 | 1.00 | 51.39 | |
| ATOM | 43 | C8 | GUA | 2 | 65.851 | 50.736 | 38.256 | 1.00 | 51.53 | |
| ATOM | 44 | C2′ | GUA | 2 | 66.046 | 47.504 | 39.600 | 1.00 | 58.61 | |
| ATOM | 45 | O2′ | GUA | 2 | 66.858 | 46.649 | 40.371 | 1.00 | 60.16 | |
| ATOM | 46 | C3′ | GUA | 2 | 65.961 | 47.048 | 38.152 | 1.00 | 61.12 | |
| ATOM | 47 | O3′ | GUA | 2 | 65.637 | 45.672 | 38.004 | 1.00 | 62.40 | |
| ATOM | 48 | P | CYT | 3 | 64.094 | 45.227 | 38.067 | 1.00 | 62.75 | |
| ATOM | 49 | O1P | CYT | 3 | 63.308 | 46.272 | 37.367 | 1.00 | 63.25 | |
| ATOM | 50 | O2P | CYT | 3 | 63.952 | 43.798 | 37.682 | 1.00 | 61.96 | |
| ATOM | 51 | O5′ | CYT | 3 | 63.702 | 45.427 | 39.599 | 1.00 | 62.40 | |
| ATOM | 52 | C5′ | CYT | 3 | 63.835 | 44.384 | 40.556 | 1.00 | 57.45 | |
| ATOM | 53 | C4′ | CYT | 3 | 63.166 | 44.797 | 41.852 | 1.00 | 54.88 | |
| ATOM | 54 | O4′ | CYT | 3 | 63.552 | 46.167 | 42.136 | 1.00 | 53.77 | |
| ATOM | 55 | C1′ | CYT | 3 | 62.501 | 46.841 | 42.816 | 1.00 | 51.14 | |
| ATOM | 56 | N1 | CYT | 3 | 62.106 | 48.038 | 42.074 | 1.00 | 48.09 | |
| ATOM | 57 | C6 | CYT | 3 | 62.565 | 48.279 | 40.819 | 1.00 | 47.60 | |
| ATOM | 58 | C2 | CYT | 3 | 61.224 | 48.927 | 42.684 | 1.00 | 47.83 | |
| ATOM | 59 | O2 | CYT | 3 | 60.828 | 48.670 | 43.813 | 1.00 | 49.37 | |
| ATOM | 60 | N3 | CYT | 3 | 60.819 | 50.034 | 42.025 | 1.00 | 45.85 | |
| ATOM | 61 | C4 | CYT | 3 | 61.265 | 50.257 | 40.793 | 1.00 | 46.99 | |
| ATOM | 62 | N4 | CYT | 3 | 60.849 | 51.345 | 40.166 | 1.00 | 46.92 | |
| ATOM | 63 | C5 | CYT | 3 | 62.169 | 49.366 | 40.145 | 1.00 | 47.49 | |
| ATOM | 64 | C2′ | CYT | 3 | 61.350 | 45.867 | 42.930 | 1.00 | 52.31 | |
| ATOM | 65 | O2′ | CYT | 3 | 61.454 | 45.180 | 44.153 | 1.00 | 53.02 | |
| ATOM | 66 | C3′ | CYT | 3 | 61.657 | 44.936 | 41.784 | 1.00 | 53.89 | |
| ATOM | 67 | O3′ | CYT | 3 | 60.969 | 43.748 | 42.011 | 1.00 | 55.88 | |
| ATOM | 68 | P | URI | 4 | 59.519 | 43.568 | 41.365 | 1.00 | 58.28 | |
| ATOM | 69 | O1P | URI | 4 | 59.598 | 44.203 | 40.021 | 1.00 | 59.11 | |
| ATOM | 70 | O2P | URI | 4 | 59.288 | 42.087 | 41.473 | 1.00 | 58.24 | |
| ATOM | 71 | O5′ | URI | 4 | 58.511 | 44.452 | 42.253 | 1.00 | 52.46 | |
| ATOM | 72 | C5′ | URI | 4 | 58.201 | 44.075 | 43.573 | 1.00 | 51.73 | |
| ATOM | 73 | C4′ | URI | 4 | 57.233 | 45.044 | 44.190 | 1.00 | 53.93 | |
| ATOM | 74 | O4′ | URI | 4 | 57.799 | 46.375 | 44.177 | 1.00 | 54.83 | |
| ATOM | 75 | C1′ | URI | 4 | 56.772 | 47.338 | 43.970 | 1.00 | 51.88 | |
| ATOM | 76 | N1 | URI | 4 | 57.043 | 48.043 | 42.715 | 1.00 | 47.56 | |
| ATOM | 77 | C6 | URI | 4 | 57.915 | 47.533 | 41.803 | 1.00 | 45.74 | |
| ATOM | 78 | C2 | URI | 4 | 56.418 | 49.246 | 42.509 | 1.00 | 45.12 | |
| ATOM | 79 | O2 | URI | 4 | 55.601 | 49.699 | 43.263 | 1.00 | 45.46 | |
| ATOM | 80 | N3 | URI | 4 | 56.780 | 49.897 | 41.376 | 1.00 | 44.07 | |
| ATOM | 81 | C4 | URI | 4 | 57.673 | 49.459 | 40.434 | 1.00 | 45.80 | |
| ATOM | 82 | O4 | URI | 4 | 57.901 | 50.160 | 39.446 | 1.00 | 48.12 | |
| ATOM | 83 | C5 | URI | 4 | 58.248 | 48.183 | 40.699 | 1.00 | 44.89 | |
| ATOM | 84 | C2′ | URI | 4 | 55.460 | 46.585 | 43.945 | 1.00 | 53.84 | |
| ATOM | 85 | O2′ | URI | 4 | 55.049 | 46.526 | 45.291 | 1.00 | 55.89 | |
| ATOM | 86 | C3′ | URI | 4 | 55.928 | 45.227 | 43.447 | 1.00 | 56.50 | |
| ATOM | 87 | O3′ | URI | 4 | 55.033 | 44.179 | 43.762 | 1.00 | 60.61 | |
| ATOM | 88 | P | URI | 5 | 54.151 | 43.529 | 42.600 | 1.00 | 60.54 | |
| ATOM | 89 | O1P | URI | 5 | 54.965 | 43.431 | 41.367 | 1.00 | 60.22 | |
| ATOM | 90 | O2P | URI | 5 | 53.623 | 42.287 | 43.225 | 1.00 | 61.03 | |
| ATOM | 91 | O5′ | URI | 5 | 53.013 | 44.623 | 42.389 | 1.00 | 57.24 | |
| ATOM | 92 | C5′ | URI | 5 | 52.117 | 44.909 | 43.441 | 1.00 | 56.41 | |
| ATOM | 93 | C4′ | URI | 5 | 51.412 | 46.192 | 43.165 | 1.00 | 56.25 | |
| ATOM | 94 | O4′ | URI | 5 | 52.393 | 47.243 | 43.070 | 1.00 | 56.34 | |
| ATOM | 95 | C1′ | URI | 5 | 52.002 | 48.171 | 42.068 | 1.00 | 54.84 | |
| ATOM | 96 | N1 | URI | 5 | 53.033 | 48.162 | 41.030 | 1.00 | 50.95 | |
| ATOM | 97 | C6 | URI | 5 | 53.904 | 47.111 | 40.940 | 1.00 | 49.39 | |
| ATOM | 98 | C2 | URI | 5 | 53.102 | 49.244 | 40.165 | 1.00 | 49.65 | |
| ATOM | 99 | O2 | URI | 5 | 52.318 | 50.188 | 40.200 | 1.00 | 49.20 | |
| ATOM | 100 | N3 | URI | 5 | 54.113 | 49.177 | 39.255 | 1.00 | 47.87 | |
| ATOM | 101 | C4 | URI | 5 | 55.025 | 48.154 | 39.116 | 1.00 | 50.19 | |
| ATOM | 102 | O4 | URI | 5 | 55.877 | 48.217 | 38.216 | 1.00 | 53.52 | |
| ATOM | 103 | C5 | URI | 5 | 54.869 | 47.069 | 40.043 | 1.00 | 49.42 | |
| ATOM | 104 | C2′ | URI | 5 | 50.646 | 47.722 | 41.541 | 1.00 | 56.19 | |
| ATOM | 105 | O2′ | URI | 5 | 49.666 | 48.386 | 42.297 | 1.00 | 58.79 | |
| ATOM | 106 | C3′ | URI | 5 | 50.698 | 46.231 | 41.837 | 1.00 | 57.01 | |
| ATOM | 107 | O3′ | URI | 5 | 49.405 | 45.691 | 41.984 | 1.00 | 59.04 | |
| ATOM | 108 | P | ADE | 6 | 48.680 | 45.048 | 40.716 | 1.00 | 61.15 | |
| ATOM | 109 | O1P | ADE | 6 | 49.746 | 44.338 | 39.944 | 1.00 | 60.00 | |
| ATOM | 110 | O2P | ADE | 6 | 47.517 | 44.282 | 41.222 | 1.00 | 62.58 | |
| ATOM | 111 | O5′ | ADE | 6 | 48.118 | 46.322 | 39.943 | 1.00 | 58.46 | |
| ATOM | 112 | C5′ | ADE | 6 | 47.075 | 47.089 | 40.511 | 1.00 | 54.84 | |
| ATOM | 113 | C4′ | ADE | 6 | 46.878 | 48.367 | 39.732 | 1.00 | 56.15 | |
| ATOM | 114 | O4′ | ADE | 6 | 48.103 | 49.137 | 39.782 | 1.00 | 55.83 | |
| ATOM | 115 | C1′ | ADE | 6 | 48.288 | 49.847 | 38.565 | 1.00 | 53.94 | |
| ATOM | 116 | N9 | ADE | 6 | 49.550 | 49.441 | 37.944 | 1.00 | 51.65 | |
| ATOM | 117 | C4 | ADE | 6 | 50.293 | 50.222 | 37.098 | 1.00 | 48.72 | |
| ATOM | 118 | N3 | ADE | 6 | 50.023 | 51.476 | 36.711 | 1.00 | 49.61 | |
| ATOM | 119 | C2 | ADE | 6 | 50.949 | 51.909 | 35.860 | 1.00 | 48.98 | |
| ATOM | 120 | N1 | ADE | 6 | 52.033 | 51.280 | 35.405 | 1.00 | 48.66 | |
| ATOM | 121 | C6 | ADE | 6 | 52.271 | 50.019 | 35.837 | 1.00 | 48.40 | |
| ATOM | 122 | N6 | ADE | 6 | 53.357 | 49.386 | 35.415 | 1.00 | 47.84 | |
| ATOM | 123 | C5 | ADE | 6 | 51.365 | 49.449 | 36.713 | 1.00 | 47.62 | |
| ATOM | 124 | N7 | ADE | 6 | 51.322 | 48.202 | 37.312 | 1.00 | 49.62 | |
| ATOM | 125 | C8 | ADE | 6 | 50.226 | 48.246 | 38.040 | 1.00 | 51.71 | |
| ATOM | 126 | C2′ | ADE | 6 | 47.082 | 49.557 | 37.691 | 1.00 | 55.35 | |
| ATOM | 127 | O2′ | ADE | 6 | 46.167 | 50.586 | 37.959 | 1.00 | 56.32 | |
| ATOM | 128 | C3′ | ADE | 6 | 46.643 | 48.207 | 38.241 | 1.00 | 57.62 | |
| ATOM | 129 | O3′ | ADE | 6 | 45.298 | 47.873 | 37.934 | 1.00 | 60.36 | |
| ATOM | 130 | P | URI | 7 | 45.013 | 46.815 | 36.761 | 1.00 | 62.82 | |
| ATOM | 131 | O1P | URI | 7 | 46.169 | 45.870 | 36.681 | 1.00 | 61.91 | |
| ATOM | 132 | O2P | URI | 7 | 43.638 | 46.278 | 36.972 | 1.00 | 63.73 | |
| ATOM | 133 | O5′ | URI | 7 | 45.037 | 47.731 | 35.464 | 1.00 | 60.87 | |
| ATOM | 134 | C5′ | URI | 7 | 44.309 | 48.943 | 35.451 | 1.00 | 59.43 | |
| ATOM | 135 | C4′ | URI | 7 | 44.805 | 49.828 | 34.343 | 1.00 | 59.36 | |
| ATOM | 136 | O4′ | URI | 7 | 46.136 | 50.328 | 34.653 | 1.00 | 57.66 | |
| ATOM | 137 | C1′ | URI | 7 | 46.887 | 50.433 | 33.470 | 1.00 | 55.50 | |
| ATOM | 138 | N1 | URI | 7 | 48.051 | 49.565 | 33.611 | 1.00 | 52.89 | |
| ATOM | 139 | C6 | URI | 7 | 48.080 | 48.541 | 34.527 | 1.00 | 52.24 | |
| ATOM | 140 | C2 | URI | 7 | 49.110 | 49.813 | 32.772 | 1.00 | 51.74 | |
| ATOM | 141 | O2 | URI | 7 | 49.081 | 50.708 | 31.942 | 1.00 | 54.38 | |
| ATOM | 142 | N3 | URI | 7 | 50.187 | 48.976 | 32.926 | 1.00 | 48.76 | |
| ATOM | 143 | C4 | URI | 7 | 50.292 | 47.913 | 33.822 | 1.00 | 49.78 | |
| ATOM | 144 | O4 | URI | 7 | 51.321 | 47.233 | 33.844 | 1.00 | 48.52 | |
| ATOM | 145 | C5 | URI | 7 | 49.140 | 47.714 | 34.655 | 1.00 | 49.80 | |
| ATOM | 146 | C2′ | URI | 7 | 45.991 | 49.967 | 32.323 | 1.00 | 57.47 | |
| ATOM | 147 | O2′ | URI | 7 | 45.309 | 51.065 | 31.766 | 1.00 | 58.56 | |
| ATOM | 148 | C3′ | URI | 7 | 45.014 | 49.073 | 33.057 | 1.00 | 58.82 | |
| ATOM | 149 | O3′ | URI | 7 | 43.790 | 48.929 | 32.393 | 1.00 | 60.27 | |
| ATOM | 150 | P | CYT | 8 | 43.577 | 47.678 | 31.430 | 1.00 | 61.88 | |
| ATOM | 151 | O1P | CYT | 8 | 44.175 | 46.480 | 32.039 | 1.00 | 61.56 | |
| ATOM | 152 | O2P | CYT | 8 | 42.136 | 47.693 | 31.094 | 1.00 | 62.68 | |
| ATOM | 153 | O5′ | CYT | 8 | 44.444 | 48.053 | 30.152 | 1.00 | 60.23 | |
| ATOM | 154 | C5′ | CYT | 8 | 44.186 | 49.260 | 29.471 | 1.00 | 58.67 | |
| ATOM | 155 | C4′ | CYT | 8 | 45.158 | 49.439 | 28.352 | 1.00 | 59.10 | |
| ATOM | 156 | O4′ | CYT | 8 | 46.464 | 49.751 | 28.899 | 1.00 | 58.92 | |
| ATOM | 157 | C1′ | CYT | 8 | 47.471 | 49.201 | 28.067 | 1.00 | 55.81 | |
| ATOM | 158 | N1 | CYT | 8 | 48.330 | 48.336 | 28.875 | 1.00 | 52.92 | |
| ATOM | 159 | C6 | CYT | 8 | 47.831 | 47.612 | 29.919 | 1.00 | 51.12 | |
| ATOM | 160 | C2 | CYT | 8 | 49.671 | 48.249 | 28.542 | 1.00 | 51.33 | |
| ATOM | 161 | O2 | CYT | 8 | 50.092 | 48.952 | 27.622 | 1.00 | 54.13 | |
| ATOM | 162 | N3 | CYT | 8 | 50.477 | 47.416 | 29.227 | 1.00 | 47.49 | |
| ATOM | 163 | C4 | CYT | 8 | 49.983 | 46.705 | 30.233 | 1.00 | 46.29 | |
| ATOM | 164 | N4 | CYT | 8 | 50.796 | 45.904 | 30.870 | 1.00 | 44.40 | |
| ATOM | 165 | C5 | CYT | 8 | 48.622 | 46.791 | 30.623 | 1.00 | 47.07 | |
| ATOM | 166 | C2′ | CYT | 8 | 46.769 | 48.478 | 26.915 | 1.00 | 57.91 | |
| ATOM | 167 | O2′ | CYT | 8 | 46.666 | 49.361 | 25.801 | 1.00 | 57.76 | |
| ATOM | 168 | C3′ | CYT | 8 | 45.405 | 48.187 | 27.531 | 1.00 | 59.65 | |
| ATOM | 169 | O3′ | CYT | 8 | 44.383 | 48.041 | 26.550 | 1.00 | 62.84 | |
| ATOM | 170 | P | ADE | 9 | 44.188 | 46.633 | 25.809 | 1.00 | 64.18 | |
| ATOM | 171 | O1P | ADE | 9 | 43.435 | 45.711 | 26.688 | 1.00 | 64.52 | |
| ATOM | 172 | O2P | ADE | 9 | 45.514 | 46.225 | 25.309 | 1.00 | 64.53 | |
| ATOM | 173 | O5′ | ADE | 9 | 43.235 | 47.012 | 24.593 | 1.00 | 66.69 | |
| ATOM | 174 | C5′ | ADE | 9 | 43.669 | 46.853 | 23.248 | 1.00 | 70.50 | |
| ATOM | 175 | C4′ | ADE | 9 | 42.669 | 47.476 | 22.294 | 1.00 | 73.26 | |
| ATOM | 176 | O4′ | ADE | 9 | 41.540 | 46.599 | 22.113 | 1.00 | 76.14 | |
| ATOM | 177 | C1′ | ADE | 9 | 40.403 | 47.114 | 22.774 | 1.00 | 79.10 | |
| ATOM | 178 | N9 | ADE | 9 | 39.929 | 46.065 | 23.673 | 1.00 | 85.08 | |
| ATOM | 179 | C4 | ADE | 9 | 39.483 | 44.834 | 23.246 | 1.00 | 88.44 | |
| ATOM | 180 | N3 | ADE | 9 | 39.407 | 44.392 | 21.975 | 1.00 | 89.70 | |
| ATOM | 181 | C2 | ADE | 9 | 38.931 | 43.140 | 21.933 | 1.00 | 91.24 | |
| ATOM | 182 | N1 | ADE | 9 | 38.552 | 42.337 | 22.945 | 1.00 | 91.09 | |
| ATOM | 183 | C6 | ADE | 9 | 38.644 | 42.808 | 24.215 | 1.00 | 90.90 | |
| ATOM | 184 | N6 | ADE | 9 | 38.275 | 42.004 | 25.220 | 1.00 | 90.36 | |
| ATOM | 185 | C5 | ADE | 9 | 39.134 | 44.135 | 24.393 | 1.00 | 89.80 | |
| ATOM | 186 | N7 | ADE | 9 | 39.355 | 44.916 | 25.525 | 1.00 | 89.22 | |
| ATOM | 187 | C8 | ADE | 9 | 39.826 | 46.049 | 25.045 | 1.00 | 86.91 | |
| ATOM | 188 | C2′ | ADE | 9 | 40.738 | 48.460 | 23.424 | 1.00 | 75.04 | |
| ATOM | 189 | O2′ | ADE | 9 | 39.712 | 49.379 | 23.103 | 1.00 | 74.28 | |
| ATOM | 190 | C3′ | ADE | 9 | 42.090 | 48.782 | 22.788 | 1.00 | 72.63 | |
| ATOM | 191 | O3′ | ADE | 9 | 42.420 | 49.947 | 22.028 | 1.00 | 69.18 | |
| ATOM | 192 | P | ADE | 10 | 41.580 | 50.305 | 20.731 | 1.00 | 61.69 | |
| ATOM | 193 | O1P | ADE | 10 | 42.493 | 50.891 | 19.750 | 1.00 | 66.22 | |
| ATOM | 194 | O2P | ADE | 10 | 40.894 | 49.052 | 20.417 | 1.00 | 64.95 | |
| ATOM | 195 | O5′ | ADE | 10 | 40.583 | 51.443 | 21.217 | 1.00 | 62.09 | |
| ATOM | 196 | C5′ | ADE | 10 | 39.175 | 51.268 | 21.154 | 1.00 | 61.89 | |
| ATOM | 197 | C4′ | ADE | 10 | 38.475 | 52.489 | 21.695 | 1.00 | 61.53 | |
| ATOM | 198 | O4′ | ADE | 10 | 38.512 | 52.476 | 23.137 | 1.00 | 61.84 | |
| ATOM | 199 | C1′ | ADE | 10 | 38.705 | 53.786 | 23.612 | 1.00 | 60.45 | |
| ATOM | 200 | N9 | ADE | 10 | 40.010 | 53.797 | 24.229 | 1.00 | 61.08 | |
| ATOM | 201 | C4 | ADE | 10 | 40.583 | 54.810 | 24.948 | 1.00 | 63.10 | |
| ATOM | 202 | N3 | ADE | 10 | 40.066 | 56.018 | 25.209 | 1.00 | 64.35 | |
| ATOM | 203 | C2 | ADE | 10 | 40.877 | 56.715 | 25.980 | 1.00 | 63.42 | |
| ATOM | 204 | N1 | ADE | 10 | 42.048 | 56.374 | 26.490 | 1.00 | 63.80 | |
| ATOM | 205 | C6 | ADE | 10 | 42.537 | 55.157 | 26.220 | 1.00 | 63.55 | |
| ATOM | 206 | N6 | ADE | 10 | 43.699 | 54.810 | 26.764 | 1.00 | 65.16 | |
| ATOM | 207 | C5 | ADE | 10 | 41.782 | 54.320 | 25.392 | 1.00 | 63.77 | |
| ATOM | 208 | N7 | ADE | 10 | 41.987 | 53.029 | 24.917 | 1.00 | 63.69 | |
| ATOM | 209 | C8 | ADE | 10 | 40.907 | 52.774 | 24.234 | 1.00 | 61.52 | |
| ATOM | 210 | C2′ | ADE | 10 | 38.644 | 54.723 | 22.416 | 1.00 | 60.15 | |
| ATOM | 211 | O2′ | ADE | 10 | 37.326 | 55.164 | 22.272 | 1.00 | 61.39 | |
| ATOM | 212 | C3′ | ADE | 10 | 39.091 | 53.806 | 21.296 | 1.00 | 60.69 | |
| ATOM | 213 | O3′ | ADE | 10 | 38.539 | 54.212 | 20.074 | 1.00 | 62.08 | |
| ATOM | 214 | P | GUA | 11 | 39.366 | 55.194 | 19.127 | 1.00 | 64.89 | |
| ATOM | 215 | O1P | GUA | 11 | 40.703 | 54.583 | 19.003 | 1.00 | 66.34 | |
| ATOM | 216 | O2P | GUA | 11 | 38.590 | 55.505 | 17.903 | 1.00 | 68.29 | |
| ATOM | 217 | O5′ | GUA | 11 | 39.495 | 56.551 | 19.946 | 1.00 | 65.10 | |
| ATOM | 218 | C5′ | GUA | 11 | 38.356 | 57.343 | 20.261 | 1.00 | 65.30 | |
| ATOM | 219 | C4′ | GUA | 11 | 38.799 | 58.558 | 21.028 | 1.00 | 65.45 | |
| ATOM | 220 | O4′ | GUA | 11 | 39.370 | 58.144 | 22.284 | 1.00 | 64.83 | |
| ATOM | 221 | C1′ | GUA | 11 | 40.478 | 58.955 | 22.597 | 1.00 | 64.44 | |
| ATOM | 222 | N9 | GUA | 11 | 41.605 | 58.050 | 22.743 | 1.00 | 64.53 | |
| ATOM | 223 | C4 | GUA | 11 | 42.679 | 58.165 | 23.607 | 1.00 | 65.59 | |
| ATOM | 224 | N3 | GUA | 11 | 42.916 | 59.179 | 24.469 | 1.00 | 65.73 | |
| ATOM | 225 | C2 | GUA | 11 | 44.016 | 58.976 | 25.164 | 1.00 | 64.82 | |
| ATOM | 226 | N2 | GUA | 11 | 44.425 | 59.871 | 26.048 | 1.00 | 68.42 | |
| ATOM | 227 | N1 | GUA | 11 | 44.807 | 57.883 | 25.041 | 1.00 | 63.97 | |
| ATOM | 228 | C6 | GUA | 11 | 44.586 | 56.835 | 24.176 | 1.00 | 63.42 | |
| ATOM | 229 | O6 | GUA | 11 | 45.372 | 55.897 | 24.154 | 1.00 | 64.36 | |
| ATOM | 230 | C5 | GUA | 11 | 43.420 | 57.027 | 23.411 | 1.00 | 64.27 | |
| ATOM | 231 | N7 | GUA | 11 | 42.840 | 56.226 | 22.440 | 1.00 | 64.47 | |
| ATOM | 232 | C8 | GUA | 11 | 41.777 | 56.877 | 22.069 | 1.00 | 63.23 | |
| ATOM | 233 | C2′ | GUA | 11 | 40.606 | 60.006 | 21.494 | 1.00 | 65.60 | |
| ATOM | 234 | O2′ | GUA | 11 | 39.979 | 61.195 | 21.889 | 1.00 | 68.32 | |
| ATOM | 235 | C3′ | GUA | 11 | 39.893 | 59.331 | 20.336 | 1.00 | 66.11 | |
| ATOM | 236 | O3′ | GUA | 11 | 39.257 | 60.293 | 19.523 | 1.00 | 67.76 | |
| ATOM | 237 | P | ADE | 12 | 39.778 | 60.539 | 18.029 | 1.00 | 72.60 | |
| ATOM | 238 | O1P | ADE | 12 | 40.480 | 59.300 | 17.596 | 1.00 | 71.76 | |
| ATOM | 239 | O2P | ADE | 12 | 38.627 | 61.058 | 17.237 | 1.00 | 72.46 | |
| ATOM | 240 | O5′ | ADE | 12 | 40.853 | 61.714 | 18.159 | 1.00 | 72.51 | |
| ATOM | 241 | C5′ | ADE | 12 | 40.499 | 62.956 | 18.760 | 1.00 | 73.62 | |
| ATOM | 242 | C4′ | ADE | 12 | 41.725 | 63.653 | 19.274 | 1.00 | 74.46 | |
| ATOM | 243 | O4′ | ADE | 12 | 42.269 | 62.918 | 20.396 | 1.00 | 73.15 | |
| ATOM | 244 | C1′ | ADE | 12 | 43.689 | 62.996 | 20.365 | 1.00 | 73.50 | |
| ATOM | 245 | N9 | ADE | 12 | 44.226 | 61.630 | 20.365 | 1.00 | 72.20 | |
| ATOM | 246 | C4 | ADE | 12 | 44.994 | 61.059 | 21.352 | 1.00 | 71.54 | |
| ATOM | 247 | N3 | ADE | 12 | 45.449 | 61.638 | 22.473 | 1.00 | 72.13 | |
| ATOM | 248 | C2 | ADE | 12 | 46.133 | 60.772 | 23.211 | 1.00 | 70.67 | |
| ATOM | 249 | N1 | ADE | 12 | 46.386 | 59.492 | 22.984 | 1.00 | 70.86 | |
| ATOM | 250 | C6 | ADE | 12 | 45.908 | 58.930 | 21.858 | 1.00 | 70.56 | |
| ATOM | 251 | N6 | ADE | 12 | 46.130 | 57.634 | 21.662 | 1.00 | 68.36 | |
| ATOM | 252 | C5 | ADE | 12 | 45.184 | 59.753 | 20.971 | 1.00 | 70.96 | |
| ATOM | 253 | N7 | ADE | 12 | 44.589 | 59.515 | 19.744 | 1.00 | 70.23 | |
| ATOM | 254 | C8 | ADE | 12 | 44.044 | 60.657 | 19.429 | 1.00 | 70.52 | |
| ATOM | 255 | C2′ | ADE | 12 | 44.076 | 63.842 | 19.150 | 1.00 | 74.01 | |
| ATOM | 256 | O2′ | ADE | 12 | 44.255 | 65.189 | 19.512 | 1.00 | 73.01 | |
| ATOM | 257 | C3′ | ADE | 12 | 42.853 | 63.678 | 18.274 | 1.00 | 76.00 | |
| ATOM | 258 | O3′ | ADE | 12 | 42.705 | 64.743 | 17.364 | 1.00 | 82.92 | |
| ATOM | 259 | P | GUA | 13 | 42.849 | 64.434 | 15.806 | 1.00 | 89.23 | |
| ATOM | 260 | O1P | GUA | 13 | 44.212 | 63.866 | 15.598 | 1.00 | 87.85 | |
| ATOM | 261 | O2P | GUA | 13 | 41.647 | 63.662 | 15.399 | 1.00 | 88.87 | |
| ATOM | 262 | O5′ | GUA | 13 | 42.835 | 65.828 | 15.049 | 1.00 | 92.65 | |
| ATOM | 263 | C5′ | GUA | 13 | 43.402 | 65.897 | 13.742 | 1.00 | 99.05 | |
| ATOM | 264 | C4′ | GUA | 13 | 43.901 | 67.285 | 13.454 | 1.00 | 102.86 | |
| ATOM | 265 | O4′ | GUA | 13 | 44.513 | 67.820 | 14.647 | 1.00 | 103.26 | |
| ATOM | 266 | C1′ | GUA | 13 | 45.600 | 68.636 | 14.278 | 1.00 | 103.85 | |
| ATOM | 267 | N9 | GUA | 13 | 46.764 | 68.225 | 15.052 | 1.00 | 103.84 | |
| ATOM | 268 | C4 | GUA | 13 | 47.591 | 69.091 | 15.711 | 1.00 | 103.47 | |
| ATOM | 269 | N3 | GUA | 13 | 47.491 | 70.432 | 15.678 | 1.00 | 103.15 | |
| ATOM | 270 | C2 | GUA | 13 | 48.394 | 71.016 | 16.429 | 1.00 | 103.15 | |
| ATOM | 271 | N2 | GUA | 13 | 48.425 | 72.353 | 16.471 | 1.00 | 102.90 | |
| ATOM | 272 | N1 | GUA | 13 | 49.329 | 70.334 | 17.183 | 1.00 | 102.58 | |
| ATOM | 273 | C6 | GUA | 13 | 49.453 | 68.947 | 17.245 | 1.00 | 102.36 | |
| ATOM | 274 | O6 | GUA | 13 | 50.322 | 68.436 | 17.977 | 1.00 | 100.97 | |
| ATOM | 275 | C5 | GUA | 13 | 48.483 | 68.300 | 16.406 | 1.00 | 103.12 | |
| ATOM | 276 | N7 | GUA | 13 | 48.247 | 66.950 | 16.146 | 1.00 | 103.65 | |
| ATOM | 277 | C8 | GUA | 13 | 47.223 | 66.954 | 15.326 | 1.00 | 103.51 | |
| ATOM | 278 | C2′ | GUA | 13 | 45.707 | 68.642 | 12.750 | 1.00 | 104.40 | |
| ATOM | 279 | O2′ | GUA | 13 | 45.031 | 69.770 | 12.214 | 1.00 | 102.71 | |
| ATOM | 280 | C3′ | GUA | 13 | 44.995 | 67.349 | 12.398 | 1.00 | 104.50 | |
| ATOM | 281 | O3′ | GUA | 13 | 44.415 | 67.468 | 11.112 | 1.00 | 108.89 | |
| ATOM | 282 | P | ADE | 14 | 44.724 | 66.356 | 10.001 | 1.00 | 112.49 | |
| ATOM | 283 | O1P | ADE | 14 | 43.489 | 65.535 | 9.857 | 1.00 | 112.37 | |
| ATOM | 284 | O2P | ADE | 14 | 46.003 | 65.706 | 10.378 | 1.00 | 112.67 | |
| ATOM | 285 | O5′ | ADE | 14 | 44.924 | 67.177 | 8.642 | 1.00 | 114.10 | |
| ATOM | 286 | C5′ | ADE | 14 | 46.180 | 67.784 | 8.294 | 1.00 | 115.83 | |
| ATOM | 287 | C4′ | ADE | 14 | 45.953 | 68.890 | 7.277 | 1.00 | 117.07 | |
| ATOM | 288 | O4′ | ADE | 14 | 45.442 | 68.287 | 6.059 | 1.00 | 118.27 | |
| ATOM | 289 | C1′ | ADE | 14 | 44.215 | 68.886 | 5.685 | 1.00 | 119.02 | |
| ATOM | 290 | N9 | ADE | 14 | 43.216 | 67.811 | 5.577 | 1.00 | 120.22 | |
| ATOM | 291 | C4 | ADE | 14 | 42.010 | 67.819 | 4.904 | 1.00 | 121.47 | |
| ATOM | 292 | N3 | ADE | 14 | 41.457 | 68.832 | 4.210 | 1.00 | 122.68 | |
| ATOM | 293 | C2 | ADE | 14 | 40.279 | 68.463 | 3.683 | 1.00 | 122.61 | |
| ATOM | 294 | N1 | ADE | 14 | 39.643 | 67.285 | 3.758 | 1.00 | 121.89 | |
| ATOM | 295 | C6 | ADE | 14 | 40.225 | 66.284 | 4.454 | 1.00 | 122.02 | |
| ATOM | 296 | N6 | ADE | 14 | 39.604 | 65.100 | 4.507 | 1.00 | 122.32 | |
| ATOM | 297 | C5 | ADE | 14 | 41.473 | 66.551 | 5.077 | 1.00 | 121.82 | |
| ATOM | 298 | N7 | ADE | 14 | 42.312 | 65.766 | 5.858 | 1.00 | 121.29 | |
| ATOM | 299 | C8 | ADE | 14 | 43.324 | 66.557 | 6.128 | 1.00 | 120.40 | |
| ATOM | 300 | C2′ | ADE | 14 | 43.866 | 70.034 | 6.651 | 1.00 | 117.90 | |
| ATOM | 301 | O2′ | ADE | 14 | 43.874 | 71.246 | 5.920 | 1.00 | 117.05 | |
| ATOM | 302 | C3′ | ADE | 14 | 44.963 | 69.962 | 7.732 | 1.00 | 116.80 | |
| ATOM | 303 | O3′ | ADE | 14 | 45.602 | 71.196 | 8.120 | 1.00 | 114.78 | |
| ATOM | 304 | P | GUA | 15 | 46.784 | 71.177 | 9.231 | 1.00 | 113.16 | |
| ATOM | 305 | O1P | GUA | 15 | 48.093 | 71.135 | 8.523 | 1.00 | 112.02 | |
| ATOM | 306 | O2P | GUA | 15 | 46.452 | 70.117 | 10.221 | 1.00 | 113.09 | |
| ATOM | 307 | O5′ | GUA | 15 | 46.685 | 72.580 | 9.983 | 1.00 | 109.53 | |
| ATOM | 308 | C5′ | GUA | 15 | 46.922 | 73.797 | 9.302 | 1.00 | 104.29 | |
| ATOM | 309 | C4′ | GUA | 15 | 47.641 | 74.768 | 10.205 | 1.00 | 101.82 | |
| ATOM | 310 | O4′ | GUA | 15 | 47.447 | 74.354 | 11.589 | 1.00 | 100.71 | |
| ATOM | 311 | C1′ | GUA | 15 | 48.608 | 74.668 | 12.349 | 1.00 | 99.42 | |
| ATOM | 312 | N9 | GUA | 15 | 49.190 | 73.447 | 12.903 | 1.00 | 98.78 | |
| ATOM | 313 | C4 | GUA | 15 | 50.201 | 73.420 | 13.825 | 1.00 | 97.48 | |
| ATOM | 314 | N3 | GUA | 15 | 50.778 | 74.498 | 14.382 | 1.00 | 97.05 | |
| ATOM | 315 | C2 | GUA | 15 | 51.734 | 74.171 | 15.222 | 1.00 | 97.04 | |
| ATOM | 316 | N2 | GUA | 15 | 52.404 | 75.129 | 15.863 | 1.00 | 96.61 | |
| ATOM | 317 | N1 | GUA | 15 | 52.104 | 72.886 | 15.496 | 1.00 | 97.43 | |
| ATOM | 318 | C6 | GUA | 15 | 51.531 | 71.753 | 14.934 | 1.00 | 97.90 | |
| ATOM | 319 | O6 | GUA | 15 | 51.956 | 70.633 | 15.250 | 1.00 | 98.14 | |
| ATOM | 320 | C5 | GUA | 15 | 50.487 | 72.092 | 14.024 | 1.00 | 97.66 | |
| ATOM | 321 | N7 | GUA | 15 | 49.655 | 71.287 | 13.251 | 1.00 | 98.14 | |
| ATOM | 322 | C8 | GUA | 15 | 48.895 | 72.135 | 12.608 | 1.00 | 98.22 | |
| ATOM | 323 | C2′ | GUA | 15 | 49.611 | 75.323 | 11.403 | 1.00 | 99.61 | |
| ATOM | 324 | O2′ | GUA | 15 | 49.544 | 76.727 | 11.480 | 1.00 | 98.50 | |
| ATOM | 325 | C3′ | GUA | 15 | 49.152 | 74.776 | 10.062 | 1.00 | 100.26 | |
| ATOM | 326 | O3′ | GUA | 15 | 49.603 | 75.614 | 9.019 | 1.00 | 99.71 | |
| ATOM | 327 | P | GUA | 16 | 51.122 | 75.491 | 8.526 | 1.00 | 99.69 | |
| ATOM | 328 | O1P | GUA | 16 | 51.687 | 74.280 | 9.172 | 1.00 | 99.91 | |
| ATOM | 329 | O2P | GUA | 16 | 51.168 | 75.611 | 7.049 | 1.00 | 100.43 | |
| ATOM | 330 | O5′ | GUA | 16 | 51.857 | 76.752 | 9.156 | 1.00 | 98.59 | |
| ATOM | 331 | C5′ | GUA | 16 | 53.234 | 76.959 | 8.897 | 1.00 | 98.52 | |
| ATOM | 332 | C4′ | GUA | 16 | 53.942 | 77.398 | 10.149 | 1.00 | 98.91 | |
| ATOM | 333 | O4′ | GUA | 16 | 53.336 | 76.758 | 11.297 | 1.00 | 98.89 | |
| ATOM | 334 | C1′ | GUA | 16 | 54.341 | 76.472 | 12.262 | 1.00 | 98.43 | |
| ATOM | 335 | N9 | GUA | 16 | 54.286 | 75.058 | 12.616 | 1.00 | 97.00 | |
| ATOM | 336 | C4 | GUA | 16 | 55.091 | 74.428 | 13.526 | 1.00 | 95.97 | |
| ATOM | 337 | N3 | GUA | 16 | 56.084 | 75.003 | 14.229 | 1.00 | 96.30 | |
| ATOM | 338 | C2 | GUA | 16 | 56.674 | 74.145 | 15.044 | 1.00 | 96.19 | |
| ATOM | 339 | N2 | GUA | 16 | 57.679 | 74.559 | 15.828 | 1.00 | 95.68 | |
| ATOM | 340 | N1 | GUA | 16 | 56.316 | 72.826 | 15.150 | 1.00 | 95.84 | |
| ATOM | 341 | C6 | GUA | 16 | 55.294 | 72.219 | 14.430 | 1.00 | 95.52 | |
| ATOM | 342 | O6 | GUA | 16 | 55.047 | 71.034 | 14.604 | 1.00 | 96.17 | |
| ATOM | 343 | C5 | GUA | 16 | 54.657 | 73.126 | 13.560 | 1.00 | 95.56 | |
| ATOM | 344 | N7 | GUA | 16 | 53.606 | 72.934 | 12.677 | 1.00 | 96.06 | |
| ATOM | 345 | C8 | GUA | 16 | 53.423 | 74.106 | 12.136 | 1.00 | 96.63 | |
| ATOM | 346 | C2′ | GUA | 16 | 55.685 | 76.926 | 11.695 | 1.00 | 98.75 | |
| ATOM | 347 | O2′ | GUA | 16 | 56.026 | 78.179 | 12.230 | 1.00 | 98.82 | |
| ATOM | 348 | C3′ | GUA | 16 | 55.392 | 76.948 | 10.202 | 1.00 | 99.86 | |
| ATOM | 349 | O3′ | GUA | 16 | 56.238 | 77.844 | 9.497 | 1.00 | 102.10 | |
| ATOM | 350 | P | URI | 17 | 57.473 | 77.262 | 8.651 | 1.00 | 103.81 | |
| ATOM | 351 | O1P | URI | 17 | 56.956 | 76.105 | 7.879 | 1.00 | 104.45 | |
| ATOM | 352 | O2P | URI | 17 | 58.117 | 78.383 | 7.927 | 1.00 | 104.70 | |
| ATOM | 353 | O5′ | URI | 17 | 58.483 | 76.767 | 9.778 | 1.00 | 102.87 | |
| ATOM | 354 | C5′ | URI | 17 | 59.132 | 77.720 | 10.590 | 1.00 | 103.52 | |
| ATOM | 355 | C4′ | URI | 17 | 60.070 | 77.052 | 11.548 | 1.00 | 104.73 | |
| ATOM | 356 | O4′ | URI | 17 | 59.300 | 76.292 | 12.514 | 1.00 | 105.61 | |
| ATOM | 357 | C1′ | URI | 17 | 60.029 | 75.135 | 12.888 | 1.00 | 106.42 | |
| ATOM | 358 | N1 | URI | 17 | 59.237 | 73.933 | 12.608 | 1.00 | 107.04 | |
| ATOM | 359 | C6 | URI | 17 | 58.117 | 73.949 | 11.812 | 1.00 | 107.16 | |
| ATOM | 360 | C2 | URI | 17 | 59.678 | 72.769 | 13.195 | 1.00 | 107.75 | |
| ATOM | 361 | O2 | URI | 17 | 60.673 | 72.727 | 13.899 | 1.00 | 107.85 | |
| ATOM | 362 | N3 | URI | 17 | 58.920 | 71.657 | 12.931 | 1.00 | 108.35 | |
| ATOM | 363 | C4 | URI | 17 | 57.790 | 71.590 | 12.154 | 1.00 | 108.47 | |
| ATOM | 364 | O4 | URI | 17 | 57.215 | 70.505 | 12.019 | 1.00 | 108.41 | |
| ATOM | 365 | C5 | URI | 17 | 57.393 | 72.846 | 11.569 | 1.00 | 108.02 | |
| ATOM | 366 | C2′ | URI | 17 | 61.344 | 75.144 | 12.115 | 1.00 | 106.06 | |
| ATOM | 367 | O2′ | URI | 17 | 62.345 | 75.727 | 12.928 | 1.00 | 106.68 | |
| ATOM | 368 | C3′ | URI | 17 | 60.973 | 76.007 | 10.921 | 1.00 | 105.15 | |
| ATOM | 369 | O3′ | URI | 17 | 62.113 | 76.578 | 10.319 | 1.00 | 104.51 | |
| ATOM | 370 | P | GUA | 18 | 62.694 | 75.923 | 8.980 | 1.00 | 104.43 | |
| ATOM | 371 | O1P | GUA | 18 | 61.537 | 75.870 | 8.046 | 1.00 | 104.17 | |
| ATOM | 372 | O2P | GUA | 18 | 63.929 | 76.658 | 8.594 | 1.00 | 105.35 | |
| ATOM | 373 | O5′ | GUA | 18 | 63.149 | 74.463 | 9.423 | 1.00 | 103.10 | |
| ATOM | 374 | C5′ | GUA | 18 | 64.238 | 74.303 | 10.329 | 1.00 | 101.95 | |
| ATOM | 375 | C4′ | GUA | 18 | 64.384 | 72.859 | 10.737 | 1.00 | 100.63 | |
| ATOM | 376 | O4′ | GUA | 18 | 63.159 | 72.422 | 11.378 | 1.00 | 100.46 | |
| ATOM | 377 | C1′ | GUA | 18 | 62.892 | 71.073 | 11.019 | 1.00 | 98.73 | |
| ATOM | 378 | N9 | GUA | 18 | 61.652 | 71.057 | 10.245 | 1.00 | 96.32 | |
| ATOM | 379 | C4 | GUA | 18 | 60.768 | 70.017 | 10.138 | 1.00 | 95.17 | |
| ATOM | 380 | N3 | GUA | 18 | 60.890 | 68.812 | 10.729 | 1.00 | 94.41 | |
| ATOM | 381 | C2 | GUA | 18 | 59.883 | 68.015 | 10.432 | 1.00 | 94.07 | |
| ATOM | 382 | N2 | GUA | 18 | 59.854 | 66.775 | 10.919 | 1.00 | 93.96 | |
| ATOM | 383 | N1 | GUA | 18 | 58.830 | 68.375 | 9.626 | 1.00 | 94.29 | |
| ATOM | 384 | C6 | GUA | 18 | 58.685 | 69.612 | 9.007 | 1.00 | 94.03 | |
| ATOM | 385 | O6 | GUA | 18 | 57.704 | 69.830 | 8.296 | 1.00 | 93.31 | |
| ATOM | 386 | C5 | GUA | 18 | 59.763 | 70.474 | 9.312 | 1.00 | 94.35 | |
| ATOM | 387 | N7 | GUA | 18 | 60.014 | 71.771 | 8.905 | 1.00 | 94.39 | |
| ATOM | 388 | C8 | GUA | 18 | 61.142 | 72.077 | 9.483 | 1.00 | 95.26 | |
| ATOM | 389 | C2′ | GUA | 18 | 64.099 | 70.574 | 10.219 | 1.00 | 98.82 | |
| ATOM | 390 | O2′ | GUA | 18 | 65.072 | 70.028 | 11.087 | 1.00 | 98.17 | |
| ATOM | 391 | C3′ | GUA | 18 | 64.597 | 71.870 | 9.605 | 1.00 | 99.71 | |
| ATOM | 392 | O3′ | GUA | 18 | 65.968 | 71.774 | 9.292 | 1.00 | 99.78 | |
| ATOM | 393 | P | GUA | 19 | 66.506 | 72.393 | 7.915 | 1.00 | 101.26 | |
| ATOM | 394 | O1P | GUA | 19 | 65.508 | 72.127 | 6.848 | 1.00 | 100.95 | |
| ATOM | 395 | O2P | GUA | 19 | 66.927 | 73.798 | 8.168 | 1.00 | 102.13 | |
| ATOM | 396 | O5′ | GUA | 19 | 67.802 | 71.510 | 7.635 | 1.00 | 99.33 | |
| ATOM | 397 | C5′ | GUA | 19 | 67.800 | 70.520 | 6.613 | 1.00 | 95.99 | |
| ATOM | 398 | C4′ | GUA | 19 | 67.089 | 69.273 | 7.090 | 1.00 | 93.08 | |
| ATOM | 399 | O4′ | GUA | 19 | 65.742 | 69.601 | 7.482 | 1.00 | 91.65 | |
| ATOM | 400 | C1′ | GUA | 19 | 64.876 | 68.547 | 7.127 | 1.00 | 89.98 | |
| ATOM | 401 | N9 | GUA | 19 | 63.792 | 69.122 | 6.347 | 1.00 | 87.52 | |
| ATOM | 402 | C4 | GUA | 19 | 62.479 | 68.735 | 6.373 | 1.00 | 86.63 | |
| ATOM | 403 | N3 | GUA | 19 | 61.981 | 67.668 | 7.028 | 1.00 | 86.22 | |
| ATOM | 404 | C2 | GUA | 19 | 60.668 | 67.587 | 6.904 | 1.00 | 85.72 | |
| ATOM | 405 | N2 | GUA | 19 | 60.010 | 66.575 | 7.475 | 1.00 | 86.95 | |
| ATOM | 406 | N1 | GUA | 19 | 59.905 | 68.489 | 6.208 | 1.00 | 84.37 | |
| ATOM | 407 | C6 | GUA | 19 | 60.396 | 69.594 | 5.532 | 1.00 | 84.59 | |
| ATOM | 408 | O6 | GUA | 19 | 59.622 | 70.345 | 4.942 | 1.00 | 83.20 | |
| ATOM | 409 | C5 | GUA | 19 | 61.804 | 69.686 | 5.641 | 1.00 | 85.73 | |
| ATOM | 410 | N7 | GUA | 19 | 62.686 | 70.617 | 5.115 | 1.00 | 86.01 | |
| ATOM | 411 | C8 | GUA | 19 | 63.855 | 70.225 | 5.540 | 1.00 | 86.60 | |
| ATOM | 412 | C2′ | GUA | 19 | 65.719 | 67.424 | 6.534 | 1.00 | 90.72 | |
| ATOM | 413 | O2′ | GUA | 19 | 66.112 | 66.586 | 7.585 | 1.00 | 93.46 | |
| ATOM | 414 | C3′ | GUA | 19 | 66.939 | 68.178 | 6.052 | 1.00 | 91.41 | |
| ATOM | 415 | O3′ | GUA | 19 | 68.052 | 67.320 | 6.215 | 1.00 | 90.30 | |
| ATOM | 416 | P | ADE | 20 | 68.088 | 65.930 | 5.446 | 1.00 | 89.56 | |
| ATOM | 417 | O1P | ADE | 20 | 67.295 | 66.170 | 4.209 | 1.00 | 90.66 | |
| ATOM | 418 | O2P | ADE | 20 | 69.494 | 65.467 | 5.355 | 1.00 | 89.16 | |
| ATOM | 419 | O5′ | ADE | 20 | 67.277 | 64.927 | 6.372 | 1.00 | 87.56 | |
| ATOM | 420 | C5′ | ADE | 20 | 67.937 | 64.034 | 7.276 | 1.00 | 85.09 | |
| ATOM | 421 | C4′ | ADE | 20 | 67.165 | 62.728 | 7.364 | 1.00 | 83.03 | |
| ATOM | 422 | O4′ | ADE | 20 | 65.756 | 63.006 | 7.576 | 1.00 | 81.17 | |
| ATOM | 423 | C1′ | ADE | 20 | 64.976 | 62.065 | 6.872 | 1.00 | 79.87 | |
| ATOM | 424 | N9 | ADE | 20 | 63.977 | 62.779 | 6.088 | 1.00 | 76.79 | |
| ATOM | 425 | C4 | ADE | 20 | 62.704 | 62.339 | 5.857 | 1.00 | 75.65 | |
| ATOM | 426 | N3 | ADE | 20 | 62.167 | 61.172 | 6.249 | 1.00 | 75.21 | |
| ATOM | 427 | C2 | ADE | 20 | 60.897 | 61.098 | 5.872 | 1.00 | 74.62 | |
| ATOM | 428 | N1 | ADE | 20 | 60.158 | 61.992 | 5.203 | 1.00 | 73.56 | |
| ATOM | 429 | C6 | ADE | 20 | 60.732 | 63.154 | 4.829 | 1.00 | 74.39 | |
| ATOM | 430 | N6 | ADE | 20 | 59.997 | 64.044 | 4.163 | 1.00 | 74.60 | |
| ATOM | 431 | C5 | ADE | 20 | 62.074 | 63.353 | 5.167 | 1.00 | 74.84 | |
| ATOM | 432 | N7 | ADE | 20 | 62.943 | 64.405 | 4.942 | 1.00 | 74.84 | |
| ATOM | 433 | C8 | ADE | 20 | 64.061 | 64.009 | 5.496 | 1.00 | 76.44 | |
| ATOM | 434 | C2′ | ADE | 20 | 65.910 | 61.114 | 6.136 | 1.00 | 81.05 | |
| ATOM | 435 | O2′ | ADE | 20 | 65.938 | 59.911 | 6.829 | 1.00 | 83.85 | |
| ATOM | 436 | C3′ | ADE | 20 | 67.204 | 61.912 | 6.091 | 1.00 | 82.02 | |
| ATOM | 437 | O3′ | ADE | 20 | 68.413 | 61.139 | 6.142 | 1.00 | 83.54 | |
| ATOM | 438 | P | GUA | 21 | 68.486 | 59.743 | 6.975 | 1.00 | 84.20 | |
| ATOM | 439 | O1P | GUA | 21 | 67.696 | 58.730 | 6.208 | 1.00 | 85.71 | |
| ATOM | 440 | O2P | GUA | 21 | 69.900 | 59.436 | 7.321 | 1.00 | 83.20 | |
| ATOM | 441 | O5′ | GUA | 21 | 67.740 | 60.043 | 8.348 | 1.00 | 80.61 | |
| ATOM | 442 | C5′ | GUA | 21 | 67.351 | 58.974 | 9.203 | 1.00 | 77.25 | |
| ATOM | 443 | C4′ | GUA | 21 | 66.008 | 59.279 | 9.810 | 1.00 | 76.64 | |
| ATOM | 444 | O4′ | GUA | 21 | 65.062 | 59.565 | 8.747 | 1.00 | 76.78 | |
| ATOM | 445 | C1′ | GUA | 21 | 63.798 | 59.027 | 9.075 | 1.00 | 76.36 | |
| ATOM | 446 | N9 | GUA | 21 | 63.594 | 57.815 | 8.286 | 1.00 | 76.44 | |
| ATOM | 447 | C4 | GUA | 21 | 62.405 | 57.150 | 8.138 | 1.00 | 76.82 | |
| ATOM | 448 | N3 | GUA | 21 | 61.229 | 57.533 | 8.661 | 1.00 | 77.21 | |
| ATOM | 449 | C2 | GUA | 21 | 60.264 | 56.688 | 8.383 | 1.00 | 77.43 | |
| ATOM | 450 | N2 | GUA | 21 | 59.038 | 56.936 | 8.852 | 1.00 | 77.66 | |
| ATOM | 451 | N1 | GUA | 21 | 60.431 | 55.542 | 7.633 | 1.00 | 77.41 | |
| ATOM | 452 | C6 | GUA | 21 | 61.631 | 55.120 | 7.077 | 1.00 | 76.61 | |
| ATOM | 453 | O6 | GUA | 21 | 61.665 | 54.070 | 6.415 | 1.00 | 74.86 | |
| ATOM | 454 | C5 | GUA | 21 | 62.693 | 56.033 | 7.384 | 1.00 | 77.01 | |
| ATOM | 455 | N7 | GUA | 21 | 64.041 | 56.005 | 7.052 | 1.00 | 77.39 | |
| ATOM | 456 | C8 | GUA | 21 | 64.532 | 57.086 | 7.598 | 1.00 | 76.36 | |
| ATOM | 457 | C2′ | GUA | 21 | 63.892 | 58.607 | 10.537 | 1.00 | 75.61 | |
| ATOM | 458 | O2′ | GUA | 21 | 63.727 | 59.739 | 11.360 | 1.00 | 75.68 | |
| ATOM | 459 | C3′ | GUA | 21 | 65.337 | 58.184 | 10.610 | 1.00 | 74.80 | |
| ATOM | 460 | O3′ | GUA | 21 | 65.724 | 58.186 | 11.942 | 1.00 | 73.18 | |
| ATOM | 461 | P | GUA | 22 | 65.505 | 56.869 | 12.802 | 1.00 | 73.59 | |
| ATOM | 462 | O1P | GUA | 22 | 66.065 | 55.721 | 12.034 | 1.00 | 72.79 | |
| ATOM | 463 | O2P | GUA | 22 | 66.030 | 57.189 | 14.160 | 1.00 | 74.63 | |
| ATOM | 464 | O5′ | GUA | 22 | 63.921 | 56.718 | 12.894 | 1.00 | 73.06 | |
| ATOM | 465 | C5′ | GUA | 22 | 63.163 | 57.673 | 13.612 | 1.00 | 71.24 | |
| ATOM | 466 | C4′ | GUA | 22 | 61.704 | 57.319 | 13.596 | 1.00 | 69.22 | |
| ATOM | 467 | O4′ | GUA | 22 | 61.263 | 57.279 | 12.212 | 1.00 | 68.19 | |
| ATOM | 468 | C1′ | GUA | 22 | 60.263 | 56.292 | 12.065 | 1.00 | 68.93 | |
| ATOM | 469 | N9 | GUA | 22 | 60.721 | 55.242 | 11.161 | 1.00 | 68.71 | |
| ATOM | 470 | C4 | GUA | 22 | 59.919 | 54.254 | 10.632 | 1.00 | 68.68 | |
| ATOM | 471 | N3 | GUA | 22 | 58.589 | 54.140 | 10.812 | 1.00 | 68.14 | |
| ATOM | 472 | C2 | GUA | 22 | 58.096 | 53.087 | 10.209 | 1.00 | 67.14 | |
| ATOM | 473 | N2 | GUA | 22 | 56.792 | 52.841 | 10.287 | 1.00 | 65.99 | |
| ATOM | 474 | N1 | GUA | 22 | 58.845 | 52.203 | 9.487 | 1.00 | 67.83 | |
| ATOM | 475 | C6 | GUA | 22 | 60.215 | 52.289 | 9.291 | 1.00 | 67.72 | |
| ATOM | 476 | O6 | GUA | 22 | 60.792 | 51.418 | 8.635 | 1.00 | 68.17 | |
| ATOM | 477 | C5 | GUA | 22 | 60.762 | 53.433 | 9.929 | 1.00 | 68.44 | |
| ATOM | 478 | N7 | GUA | 22 | 62.069 | 53.906 | 9.985 | 1.00 | 69.13 | |
| ATOM | 479 | C8 | GUA | 22 | 61.991 | 54.988 | 10.718 | 1.00 | 68.72 | |
| ATOM | 480 | C2′ | GUA | 22 | 60.025 | 55.692 | 13.449 | 1.00 | 68.69 | |
| ATOM | 481 | O2′ | GUA | 22 | 59.026 | 56.449 | 14.083 | 1.00 | 68.16 | |
| ATOM | 482 | C3′ | GUA | 22 | 61.367 | 55.931 | 14.107 | 1.00 | 68.74 | |
| ATOM | 483 | O3′ | GUA | 22 | 61.272 | 55.844 | 15.515 | 1.00 | 68.59 | |
| ATOM | 484 | P | GUA | 23 | 61.522 | 54.427 | 16.236 | 1.00 | 67.90 | |
| ATOM | 485 | O1P | GUA | 23 | 62.634 | 53.778 | 15.515 | 1.00 | 68.57 | |
| ATOM | 486 | O2P | GUA | 23 | 61.648 | 54.697 | 17.687 | 1.00 | 68.81 | |
| ATOM | 487 | O5′ | GUA | 23 | 60.173 | 53.619 | 15.992 | 1.00 | 67.18 | |
| ATOM | 488 | C5′ | GUA | 23 | 58.944 | 54.283 | 16.218 | 1.00 | 68.07 | |
| ATOM | 489 | C4′ | GUA | 23 | 57.746 | 53.406 | 15.927 | 1.00 | 67.20 | |
| ATOM | 490 | O4′ | GUA | 23 | 57.467 | 53.381 | 14.501 | 1.00 | 67.66 | |
| ATOM | 491 | C1′ | GUA | 23 | 56.933 | 52.122 | 14.143 | 1.00 | 65.47 | |
| ATOM | 492 | N9 | GUA | 23 | 57.837 | 51.462 | 13.211 | 1.00 | 65.19 | |
| ATOM | 493 | C4 | GUA | 23 | 57.510 | 50.400 | 12.405 | 1.00 | 63.86 | |
| ATOM | 494 | N3 | GUA | 23 | 56.288 | 49.857 | 12.284 | 1.00 | 63.26 | |
| ATOM | 495 | C2 | GUA | 23 | 56.289 | 48.816 | 11.493 | 1.00 | 61.70 | |
| ATOM | 496 | N2 | GUA | 23 | 55.142 | 48.160 | 11.262 | 1.00 | 60.99 | |
| ATOM | 497 | N1 | GUA | 23 | 57.403 | 48.343 | 10.875 | 1.00 | 61.92 | |
| ATOM | 498 | C6 | GUA | 23 | 58.673 | 48.881 | 10.987 | 1.00 | 62.91 | |
| ATOM | 499 | O6 | GUA | 23 | 59.619 | 48.354 | 10.388 | 1.00 | 64.21 | |
| ATOM | 500 | C5 | GUA | 23 | 58.684 | 50.006 | 11.824 | 1.00 | 63.63 | |
| ATOM | 501 | N7 | GUA | 23 | 59.727 | 50.841 | 12.205 | 1.00 | 65.20 | |
| ATOM | 502 | C8 | GUA | 23 | 59.173 | 51.702 | 13.014 | 1.00 | 65.06 | |
| ATOM | 503 | C2′ | GUA | 23 | 56.785 | 51.322 | 15.434 | 1.00 | 65.23 | |
| ATOM | 504 | O2′ | GUA | 23 | 55.523 | 51.603 | 15.988 | 1.00 | 63.62 | |
| ATOM | 505 | C3′ | GUA | 23 | 57.860 | 51.948 | 16.307 | 1.00 | 67.14 | |
| ATOM | 506 | O3′ | GUA | 23 | 57.584 | 51.754 | 17.683 | 1.00 | 69.44 | |
| ATOM | 507 | P | ADE | 24 | 58.185 | 50.479 | 18.419 | 1.00 | 70.19 | |
| ATOM | 508 | O1P | ADE | 24 | 57.107 | 49.796 | 19.167 | 1.00 | 71.17 | |
| ATOM | 509 | O2P | ADE | 24 | 58.971 | 49.724 | 17.418 | 1.00 | 72.90 | |
| ATOM | 510 | O5′ | ADE | 24 | 59.210 | 51.099 | 19.456 | 1.00 | 72.19 | |
| ATOM | 511 | C5′ | ADE | 24 | 60.399 | 51.763 | 19.027 | 1.00 | 70.68 | |
| ATOM | 512 | C4′ | ADE | 24 | 61.574 | 51.256 | 19.829 | 1.00 | 68.54 | |
| ATOM | 513 | O4′ | ADE | 24 | 61.091 | 50.862 | 21.135 | 1.00 | 66.35 | |
| ATOM | 514 | C1′ | ADE | 24 | 61.790 | 49.729 | 21.592 | 1.00 | 63.51 | |
| ATOM | 515 | N9 | ADE | 24 | 60.810 | 48.689 | 21.834 | 1.00 | 59.62 | |
| ATOM | 516 | C4 | ADE | 24 | 61.001 | 47.522 | 22.524 | 1.00 | 58.95 | |
| ATOM | 517 | N3 | ADE | 24 | 62.140 | 47.079 | 23.068 | 1.00 | 59.41 | |
| ATOM | 518 | C2 | ADE | 24 | 61.931 | 45.919 | 23.706 | 1.00 | 58.50 | |
| ATOM | 519 | N1 | ADE | 24 | 60.797 | 45.221 | 23.859 | 1.00 | 55.98 | |
| ATOM | 520 | C6 | ADE | 24 | 59.675 | 45.703 | 23.300 | 1.00 | 56.73 | |
| ATOM | 521 | N6 | ADE | 24 | 58.541 | 45.021 | 23.478 | 1.00 | 57.38 | |
| ATOM | 522 | C5 | ADE | 24 | 59.767 | 46.906 | 22.577 | 1.00 | 56.87 | |
| ATOM | 523 | N7 | ADE | 24 | 58.830 | 47.652 | 21.894 | 1.00 | 58.63 | |
| ATOM | 524 | C8 | ADE | 24 | 59.505 | 48.693 | 21.463 | 1.00 | 58.72 | |
| ATOM | 525 | C2′ | ADE | 24 | 62.838 | 49.392 | 20.543 | 1.00 | 67.29 | |
| ATOM | 526 | O2′ | ADE | 24 | 64.031 | 50.065 | 20.892 | 1.00 | 68.64 | |
| ATOM | 527 | C3′ | ADE | 24 | 62.208 | 49.990 | 19.294 | 1.00 | 68.98 | |
| ATOM | 528 | O3′ | ADE | 24 | 63.204 | 50.356 | 18.348 | 1.00 | 71.46 | |
| ATOM | 529 | P | CYT | 25 | 63.593 | 49.332 | 17.193 | 1.00 | 71.71 | |
| ATOM | 530 | O1P | CYT | 25 | 62.341 | 49.007 | 16.470 | 1.00 | 74.40 | |
| ATOM | 531 | O2P | CYT | 25 | 64.749 | 49.850 | 16.440 | 1.00 | 72.40 | |
| ATOM | 532 | O5′ | CYT | 25 | 64.023 | 48.046 | 18.013 | 1.00 | 71.74 | |
| ATOM | 533 | C5′ | CYT | 25 | 65.300 | 47.977 | 18.595 | 1.00 | 72.65 | |
| ATOM | 534 | C4′ | CYT | 25 | 65.499 | 46.633 | 19.229 | 1.00 | 74.27 | |
| ATOM | 535 | O4′ | CYT | 25 | 64.462 | 46.425 | 20.227 | 1.00 | 73.08 | |
| ATOM | 536 | C1′ | CYT | 25 | 64.150 | 45.049 | 20.290 | 1.00 | 72.40 | |
| ATOM | 537 | N1 | CYT | 25 | 62.704 | 44.865 | 20.212 | 1.00 | 71.02 | |
| ATOM | 538 | C6 | CYT | 25 | 61.868 | 45.821 | 19.708 | 1.00 | 69.74 | |
| ATOM | 539 | C2 | CYT | 25 | 62.203 | 43.678 | 20.678 | 1.00 | 70.93 | |
| ATOM | 540 | O2 | CYT | 25 | 62.999 | 42.837 | 21.094 | 1.00 | 73.35 | |
| ATOM | 541 | N3 | CYT | 25 | 60.876 | 43.456 | 20.663 | 1.00 | 70.14 | |
| ATOM | 542 | C4 | CYT | 25 | 60.061 | 44.378 | 20.184 | 1.00 | 69.58 | |
| ATOM | 543 | N4 | CYT | 25 | 58.758 | 44.097 | 20.194 | 1.00 | 70.12 | |
| ATOM | 544 | C5 | CYT | 25 | 60.546 | 45.620 | 19.677 | 1.00 | 68.57 | |
| ATOM | 545 | C2′ | CYT | 25 | 64.940 | 44.326 | 19.197 | 1.00 | 74.57 | |
| ATOM | 546 | O2′ | CYT | 25 | 66.064 | 43.676 | 19.747 | 1.00 | 74.57 | |
| ATOM | 547 | C3′ | CYT | 25 | 65.294 | 45.479 | 18.264 | 1.00 | 75.99 | |
| ATOM | 548 | O3′ | CYT | 25 | 66.497 | 45.229 | 17.547 | 1.00 | 80.43 | |
| ATOM | 549 | P | URI | 26 | 66.445 | 44.414 | 16.159 | 1.00 | 84.24 | |
| ATOM | 500 | O1P | URI | 26 | 65.626 | 45.207 | 15.207 | 1.00 | 84.42 | |
| ATOM | 551 | O2P | URI | 26 | 67.846 | 44.052 | 15.794 | 1.00 | 83.16 | |
| ATOM | 552 | O5′ | URI | 26 | 65.660 | 43.077 | 16.531 | 1.00 | 81.54 | |
| ATOM | 553 | C5′ | URI | 26 | 66.343 | 42.032 | 17.194 | 1.00 | 81.62 | |
| ATOM | 554 | C4′ | URI | 26 | 65.695 | 40.706 | 16.905 | 1.00 | 81.45 | |
| ATOM | 555 | O4′ | URI | 26 | 64.533 | 40.520 | 17.745 | 1.00 | 81.80 | |
| ATOM | 556 | C1′ | URI | 26 | 63.547 | 39.794 | 17.043 | 1.00 | 82.17 | |
| ATOM | 557 | N1 | URI | 26 | 62.302 | 40.574 | 17.025 | 1.00 | 83.08 | |
| ATOM | 558 | C6 | URI | 26 | 62.313 | 41.946 | 16.936 | 1.00 | 83.24 | |
| ATOM | 559 | C2 | URI | 26 | 61.107 | 39.869 | 17.114 | 1.00 | 83.66 | |
| ATOM | 560 | O2 | URI | 26 | 61.055 | 38.644 | 17.175 | 1.00 | 83.04 | |
| ATOM | 561 | N3 | URI | 26 | 59.976 | 40.649 | 17.131 | 1.00 | 84.64 | |
| ATOM | 562 | C4 | URI | 26 | 59.917 | 42.031 | 17.060 | 1.00 | 85.35 | |
| ATOM | 563 | O4 | URI | 26 | 58.815 | 42.592 | 17.096 | 1.00 | 86.24 | |
| ATOM | 564 | C5 | URI | 26 | 61.193 | 42.681 | 16.950 | 1.00 | 83.94 | |
| ATOM | 565 | C2′ | URI | 26 | 64.116 | 39.472 | 15.664 | 1.00 | 81.60 | |
| ATOM | 566 | O2′ | URI | 26 | 64.658 | 38.175 | 15.730 | 1.00 | 81.97 | |
| ATOM | 567 | C3′ | URI | 26 | 65.160 | 40.568 | 15.502 | 1.00 | 80.63 | |
| ATOM | 568 | O3′ | URI | 26 | 66.208 | 40.201 | 14.639 | 1.00 | 79.96 | |
| ATOM | 569 | P | GUA | 27 | 66.076 | 40.502 | 13.082 | 1.00 | 80.19 | |
| ATOM | 570 | O1P | GUA | 27 | 65.823 | 41.950 | 12.916 | 1.00 | 81.56 | |
| ATOM | 571 | O2P | GUA | 27 | 67.239 | 39.905 | 12.399 | 1.00 | 82.09 | |
| ATOM | 572 | O5′ | GUA | 27 | 64.772 | 39.677 | 12.692 | 1.00 | 79.08 | |
| ATOM | 573 | C5′ | GUA | 27 | 64.809 | 38.262 | 12.628 | 1.00 | 76.84 | |
| ATOM | 574 | C4′ | GUA | 27 | 63.456 | 37.710 | 12.238 | 1.00 | 77.25 | |
| ATOM | 575 | O4′ | GUA | 27 | 62.532 | 37.841 | 13.361 | 1.00 | 77.35 | |
| ATOM | 576 | C1′ | GUA | 27 | 61.223 | 38.126 | 12.871 | 1.00 | 75.76 | |
| ATOM | 577 | N9 | GUA | 27 | 60.851 | 39.492 | 13.263 | 1.00 | 74.07 | |
| ATOM | 578 | C4 | GUA | 27 | 59.578 | 39.967 | 13.524 | 1.00 | 72.40 | |
| ATOM | 579 | N3 | GUA | 27 | 58.441 | 39.245 | 13.512 | 1.00 | 71.17 | |
| ATOM | 580 | C2 | GUA | 27 | 57.385 | 39.985 | 13.779 | 1.00 | 69.88 | |
| ATOM | 581 | N2 | GUA | 27 | 56.182 | 39.418 | 13.811 | 1.00 | 69.05 | |
| ATOM | 582 | N1 | GUA | 27 | 57.438 | 41.328 | 14.034 | 1.00 | 69.69 | |
| ATOM | 583 | C6 | GUA | 27 | 58.600 | 42.094 | 14.055 | 1.00 | 70.63 | |
| ATOM | 584 | O6 | GUA | 27 | 58.546 | 43.314 | 14.302 | 1.00 | 70.38 | |
| ATOM | 585 | C5 | GUA | 27 | 59.736 | 41.312 | 13.774 | 1.00 | 71.53 | |
| ATOM | 586 | N7 | GUA | 27 | 61.073 | 41.673 | 13.700 | 1.00 | 72.68 | |
| ATOM | 587 | C8 | GUA | 27 | 61.696 | 40.566 | 13.401 | 1.00 | 73.18 | |
| ATOM | 588 | C2′ | GUA | 27 | 61.290 | 37.994 | 11.344 | 1.00 | 75.53 | |
| ATOM | 589 | O2′ | GUA | 27 | 61.054 | 36.659 | 10.969 | 1.00 | 73.69 | |
| ATOM | 590 | C3′ | GUA | 27 | 62.735 | 38.375 | 11.065 | 1.00 | 75.98 | |
| ATOM | 591 | O3′ | GUA | 27 | 63.174 | 37.862 | 9.818 | 1.00 | 75.44 | |
| ATOM | 592 | P | GUA | 28 | 62.453 | 38.316 | 8.446 | 1.00 | 76.14 | |
| ATOM | 593 | O1P | GUA | 28 | 63.079 | 39.549 | 7.865 | 1.00 | 75.21 | |
| ATOM | 594 | O2P | GUA | 28 | 62.400 | 37.070 | 7.624 | 1.00 | 76.48 | |
| ATOM | 595 | O5′ | GUA | 28 | 60.963 | 38.690 | 8.867 | 1.00 | 75.39 | |
| ATOM | 596 | C5′ | GUA | 28 | 60.168 | 39.575 | 8.057 | 1.00 | 73.20 | |
| ATOM | 597 | C4′ | GUA | 28 | 58.703 | 39.460 | 8.432 | 1.00 | 70.70 | |
| ATOM | 598 | O4′ | GUA | 28 | 58.540 | 39.738 | 9.850 | 1.00 | 69.93 | |
| ATOM | 599 | C1′ | GUA | 28 | 57.335 | 40.450 | 10.059 | 1.00 | 69.50 | |
| ATOM | 600 | N9 | GUA | 28 | 57.667 | 41.791 | 10.530 | 1.00 | 68.49 | |
| ATOM | 601 | C4 | GUA | 28 | 56.800 | 42.695 | 11.085 | 1.00 | 67.50 | |
| ATOM | 602 | N3 | GUA | 28 | 55.501 | 42.484 | 11.327 | 1.00 | 68.00 | |
| ATOM | 603 | C2 | GUA | 28 | 54.918 | 43.541 | 11.841 | 1.00 | 67.16 | |
| ATOM | 604 | N2 | GUA | 28 | 53.606 | 43.497 | 12.119 | 1.00 | 66.54 | |
| ATOM | 605 | N1 | GUA | 28 | 55.570 | 44.709 | 12.114 | 1.00 | 67.40 | |
| ATOM | 606 | C6 | GUA | 28 | 56.917 | 44.947 | 11.882 | 1.00 | 67.49 | |
| ATOM | 607 | O6 | GUA | 28 | 57.418 | 46.043 | 12.186 | 1.00 | 67.56 | |
| ATOM | 608 | C5 | GUA | 28 | 57.542 | 43.825 | 11.310 | 1.00 | 67.56 | |
| ATOM | 609 | N7 | GUA | 28 | 58.856 | 43.634 | 10.920 | 1.00 | 68.23 | |
| ATOM | 610 | C8 | GUA | 28 | 58.885 | 42.410 | 10.473 | 1.00 | 68.51 | |
| ATOM | 611 | C2′ | GUA | 28 | 56.599 | 40.488 | 8.718 | 1.00 | 68.94 | |
| ATOM | 612 | O2′ | GUA | 28 | 55.767 | 39.355 | 8.647 | 1.00 | 68.84 | |
| ATOM | 613 | C3′ | GUA | 28 | 57.758 | 40.420 | 7.728 | 1.00 | 69.46 | |
| ATOM | 614 | O3′ | GUA | 28 | 57.341 | 39.854 | 6.481 | 1.00 | 68.96 | |
| ATOM | 615 | P | CYT | 29 | 57.217 | 40.775 | 5.161 | 1.00 | 67.04 | |
| ATOM | 616 | O1P | CYT | 29 | 58.399 | 41.654 | 5.027 | 1.00 | 68.44 | |
| ATOM | 617 | O2P | CYT | 29 | 56.834 | 39.886 | 4.050 | 1.00 | 69.45 | |
| ATOM | 618 | O5′ | CYT | 29 | 55.982 | 41.723 | 5.427 | 1.00 | 65.84 | |
| ATOM | 619 | C5′ | CYT | 29 | 54.705 | 41.194 | 5.670 | 1.00 | 62.81 | |
| ATOM | 620 | C4′ | CYT | 29 | 53.889 | 42.234 | 6.364 | 1.00 | 62.68 | |
| ATOM | 621 | O4′ | CYT | 29 | 54.432 | 42.442 | 7.697 | 1.00 | 60.72 | |
| ATOM | 622 | C1′ | CYT | 29 | 54.326 | 43.806 | 8.039 | 1.00 | 60.35 | |
| ATOM | 623 | N1 | CYT | 29 | 55.668 | 44.354 | 8.161 | 1.00 | 60.32 | |
| ATOM | 624 | C6 | CYT | 29 | 56.759 | 43.728 | 7.613 | 1.00 | 60.56 | |
| ATOM | 625 | C2 | CYT | 29 | 55.811 | 45.553 | 8.831 | 1.00 | 60.02 | |
| ATOM | 626 | O2 | CYT | 29 | 54.815 | 46.067 | 9.319 | 1.00 | 61.68 | |
| ATOM | 627 | N3 | CYT | 29 | 57.024 | 46.128 | 8.931 | 1.00 | 60.30 | |
| ATOM | 628 | C4 | CYT | 29 | 58.089 | 45.534 | 8.385 | 1.00 | 60.53 | |
| ATOM | 629 | N4 | CYT | 29 | 59.263 | 46.156 | 8.495 | 1.00 | 58.64 | |
| ATOM | 630 | C5 | CYT | 29 | 57.986 | 44.278 | 7.699 | 1.00 | 60.04 | |
| ATOM | 631 | C2′ | CYT | 29 | 53.636 | 44.510 | 6.878 | 1.00 | 61.87 | |
| ATOM | 632 | O2′ | CYT | 29 | 52.229 | 44.511 | 7.048 | 1.00 | 62.92 | |
| ATOM | 633 | C3′ | CYT | 29 | 54.016 | 43.603 | 5.728 | 1.00 | 62.72 | |
| ATOM | 634 | O3′ | CYT | 29 | 53.172 | 43.794 | 4.608 | 1.00 | 64.21 | |
| ATOM | 635 | P | CYT | 30 | 53.207 | 45.204 | 3.837 | 1.00 | 65.43 | |
| ATOM | 636 | O1P | CYT | 30 | 54.597 | 45.546 | 3.412 | 1.00 | 63.18 | |
| ATOM | 637 | O2P | CYT | 30 | 52.095 | 45.220 | 2.862 | 1.00 | 65.26 | |
| ATOM | 638 | O5′ | CYT | 30 | 52.832 | 46.261 | 4.954 | 1.00 | 65.67 | |
| ATOM | 639 | C5′ | CYT | 30 | 52.987 | 47.636 | 4.693 | 1.00 | 64.90 | |
| ATOM | 640 | C4′ | CYT | 30 | 52.685 | 48.420 | 5.922 | 1.00 | 63.34 | |
| ATOM | 641 | O4′ | CYT | 30 | 53.471 | 47.910 | 7.018 | 1.00 | 63.18 | |
| ATOM | 642 | C1′ | CYT | 30 | 53.911 | 48.981 | 7.813 | 1.00 | 62.94 | |
| ATOM | 643 | N1 | CYT | 30 | 55.361 | 49.052 | 7.623 | 1.00 | 63.65 | |
| ATOM | 644 | C6 | CYT | 30 | 55.980 | 48.252 | 6.705 | 1.00 | 64.39 | |
| ATOM | 645 | C2 | CYT | 30 | 56.091 | 49.952 | 8.365 | 1.00 | 64.01 | |
| ATOM | 646 | O2 | CYT | 30 | 55.507 | 50.614 | 9.217 | 1.00 | 65.35 | |
| ATOM | 647 | N3 | CYT | 30 | 57.421 | 50.074 | 8.142 | 1.00 | 63.66 | |
| ATOM | 648 | C4 | CYT | 30 | 58.014 | 49.306 | 7.231 | 1.00 | 63.21 | |
| ATOM | 649 | N4 | CYT | 30 | 59.309 | 49.457 | 7.042 | 1.00 | 64.07 | |
| ATOM | 650 | C5 | CYT | 30 | 57.296 | 48.347 | 6.479 | 1.00 | 63.45 | |
| ATOM | 651 | C2′ | CYT | 30 | 53.184 | 50.229 | 7.297 | 1.00 | 63.31 | |
| ATOM | 652 | O2′ | CYT | 30 | 51.896 | 50.279 | 7.875 | 1.00 | 63.35 | |
| ATOM | 653 | C3′ | CYT | 30 | 53.007 | 49.886 | 5.833 | 1.00 | 62.69 | |
| ATOM | 654 | O3′ | CYT | 30 | 51.867 | 50.520 | 5.290 | 1.00 | 62.32 | |
| ATOM | 655 | P | CYT | 31 | 52.051 | 51.748 | 4.283 | 1.00 | 63.51 | |
| ATOM | 656 | O1P | CYT | 31 | 53.215 | 51.463 | 3.410 | 1.00 | 63.63 | |
| ATOM | 657 | O2P | CYT | 31 | 50.736 | 52.121 | 3.692 | 1.00 | 61.59 | |
| ATOM | 658 | O5′ | CYT | 31 | 52.483 | 52.922 | 5.242 | 1.00 | 63.17 | |
| ATOM | 659 | C5′ | CYT | 31 | 51.632 | 53.306 | 6.285 | 1.00 | 62.30 | |
| ATOM | 660 | C4′ | CYT | 31 | 52.314 | 54.332 | 7.105 | 1.00 | 62.39 | |
| ATOM | 661 | O4′ | CYT | 31 | 53.366 | 53.696 | 7.876 | 1.00 | 63.41 | |
| ATOM | 662 | C1′ | CYT | 31 | 54.450 | 54.591 | 7.996 | 1.00 | 62.48 | |
| ATOM | 663 | N1 | CYT | 31 | 55.653 | 53.959 | 7.458 | 1.00 | 63.75 | |
| ATOM | 664 | C6 | CYT | 31 | 55.600 | 52.796 | 6.743 | 1.00 | 64.90 | |
| ATOM | 665 | C2 | CYT | 31 | 56.856 | 54.585 | 7.677 | 1.00 | 63.38 | |
| ATOM | 666 | O2 | CYT | 31 | 56.863 | 55.604 | 8.351 | 1.00 | 64.21 | |
| ATOM | 667 | N3 | CYT | 31 | 57.981 | 54.075 | 7.162 | 1.00 | 63.74 | |
| ATOM | 668 | C4 | CYT | 31 | 57.937 | 52.958 | 6.458 | 1.00 | 64.70 | |
| ATOM | 669 | N4 | CYT | 31 | 59.086 | 52.499 | 5.969 | 1.00 | 66.48 | |
| ATOM | 670 | C5 | CYT | 31 | 56.714 | 52.265 | 6.224 | 1.00 | 65.43 | |
| ATOM | 671 | C2′ | CYT | 31 | 54.073 | 55.861 | 7.232 | 1.00 | 61.19 | |
| ATOM | 672 | O2′ | CYT | 31 | 53.439 | 56.721 | 8.140 | 1.00 | 62.33 | |
| ATOM | 673 | C3′ | CYT | 31 | 53.048 | 55.338 | 6.252 | 1.00 | 60.44 | |
| ATOM | 674 | O3′ | CYT | 31 | 52.145 | 56.337 | 5.825 | 1.00 | 60.08 | |
| ATOM | 675 | P | GUA | 32 | 52.438 | 57.157 | 4.466 | 1.00 | 61.96 | |
| ATOM | 676 | O1P | GUA | 32 | 52.699 | 56.227 | 3.338 | 1.00 | 59.37 | |
| ATOM | 677 | O2P | GUA | 32 | 51.401 | 58.205 | 4.329 | 1.00 | 60.13 | |
| ATOM | 678 | O5′ | GUA | 32 | 53.798 | 57.926 | 4.772 | 1.00 | 62.63 | |
| ATOM | 679 | C5′ | GUA | 32 | 53.783 | 59.103 | 5.553 | 1.00 | 61.55 | |
| ATOM | 680 | C4′ | GUA | 32 | 55.153 | 59.690 | 5.621 | 1.00 | 62.66 | |
| ATOM | 681 | O4′ | GUA | 32 | 56.024 | 58.773 | 6.331 | 1.00 | 64.11 | |
| ATOM | 682 | C1′ | GUA | 32 | 57.334 | 58.848 | 5.804 | 1.00 | 64.32 | |
| ATOM | 683 | N9 | GUA | 32 | 57.719 | 57.539 | 5.309 | 1.00 | 66.27 | |
| ATOM | 684 | C4 | GUA | 32 | 58.985 | 57.147 | 4.940 | 1.00 | 67.79 | |
| ATOM | 685 | N3 | GUA | 32 | 60.117 | 57.881 | 5.057 | 1.00 | 68.93 | |
| ATOM | 686 | C2 | GUA | 32 | 61.172 | 57.242 | 4.579 | 1.00 | 68.89 | |
| ATOM | 687 | N2 | GUA | 32 | 62.394 | 57.793 | 4.657 | 1.00 | 68.43 | |
| ATOM | 688 | N1 | GUA | 32 | 61.108 | 56.010 | 3.997 | 1.00 | 69.08 | |
| ATOM | 689 | C6 | GUA | 32 | 59.952 | 55.259 | 3.844 | 1.00 | 69.01 | |
| ATOM | 690 | O6 | GUA | 32 | 59.996 | 54.192 | 3.242 | 1.00 | 71.45 | |
| ATOM | 691 | C5 | GUA | 32 | 58.834 | 55.895 | 4.407 | 1.00 | 68.37 | |
| ATOM | 692 | N7 | GUA | 32 | 57.517 | 55.469 | 4.511 | 1.00 | 68.89 | |
| ATOM | 693 | C8 | GUA | 32 | 56.894 | 56.477 | 5.056 | 1.00 | 67.91 | |
| ATOM | 694 | C2′ | GUA | 32 | 57.290 | 59.857 | 4.660 | 1.00 | 64.45 | |
| ATOM | 695 | O2′ | GUA | 32 | 57.625 | 61.135 | 5.150 | 1.00 | 64.74 | |
| ATOM | 696 | C3′ | GUA | 32 | 55.826 | 59.816 | 4.275 | 1.00 | 63.92 | |
| ATOM | 697 | O3′ | GUA | 32 | 55.448 | 60.990 | 3.585 | 1.00 | 64.55 | |
| ATOM | 698 | P | ADE | 33 | 55.135 | 60.897 | 2.015 | 1.00 | 66.33 | |
| ATOM | 699 | O1P | ADE | 33 | 56.000 | 59.795 | 1.510 | 1.00 | 66.06 | |
| ATOM | 700 | O2P | ADE | 33 | 53.658 | 60.829 | 1.819 | 1.00 | 64.65 | |
| ATOM | 701 | O5′ | ADE | 33 | 55.604 | 62.294 | 1.398 | 1.00 | 66.86 | |
| ATOM | 702 | C5′ | ADE | 33 | 56.921 | 62.837 | 1.571 | 1.00 | 68.49 | |
| ATOM | 703 | C4′ | ADE | 33 | 57.072 | 64.030 | 0.642 | 1.00 | 70.20 | |
| ATOM | 704 | O4′ | ADE | 33 | 58.270 | 64.788 | 0.944 | 1.00 | 70.84 | |
| ATOM | 705 | C1′ | ADE | 33 | 59.229 | 64.641 | −0.090 | 1.00 | 70.83 | |
| ATOM | 706 | N9 | ADE | 33 | 60.542 | 64.376 | 0.506 | 1.00 | 70.15 | |
| ATOM | 707 | C4 | ADE | 33 | 61.013 | 63.207 | 1.054 | 1.00 | 70.13 | |
| ATOM | 708 | N3 | ADE | 33 | 60.367 | 62.042 | 1.169 | 1.00 | 69.93 | |
| ATOM | 709 | C2 | ADE | 33 | 61.154 | 61.127 | 1.755 | 1.00 | 70.28 | |
| ATOM | 710 | N1 | ADE | 33 | 62.408 | 61.238 | 2.200 | 1.00 | 68.00 | |
| ATOM | 711 | C6 | ADE | 33 | 63.030 | 62.423 | 2.068 | 1.00 | 69.01 | |
| ATOM | 712 | N6 | ADE | 33 | 64.283 | 62.540 | 2.511 | 1.00 | 69.69 | |
| ATOM | 713 | C5 | ADE | 33 | 62.313 | 63.475 | 1.466 | 1.00 | 70.47 | |
| ATOM | 714 | N7 | ADE | 33 | 62.655 | 64.789 | 1.185 | 1.00 | 70.34 | |
| ATOM | 715 | C8 | ADE | 33 | 61.575 | 65.278 | 0.617 | 1.00 | 70.32 | |
| ATOM | 716 | C2′ | ADE | 33 | 58.716 | 63.653 | −1.137 | 1.00 | 70.97 | |
| ATOM | 717 | O2′ | ADE | 33 | 58.937 | 64.159 | −2.436 | 1.00 | 70.72 | |
| ATOM | 718 | C3′ | ADE | 33 | 57.236 | 63.601 | −0.802 | 1.00 | 70.98 | |
| ATOM | 719 | O3′ | ADE | 33 | 56.173 | 63.738 | −1.728 | 1.00 | 72.72 | |
| ATOM | 720 | P | URI | 34 | 55.823 | 65.171 | −2.319 | 1.00 | 72.54 | |
| ATOM | 721 | O1P | URI | 34 | 54.661 | 65.082 | −3.220 | 1.00 | 74.35 | |
| ATOM | 722 | O2P | URI | 34 | 57.080 | 65.774 | −2.790 | 1.00 | 76.43 | |
| ATOM | 723 | O5′ | URI | 34 | 55.312 | 65.977 | −1.061 | 1.00 | 77.55 | |
| ATOM | 724 | C5′ | URI | 34 | 54.493 | 65.351 | −0.097 | 1.00 | 81.27 | |
| ATOM | 725 | C4′ | URI | 34 | 53.054 | 65.656 | −0.363 | 1.00 | 83.26 | |
| ATOM | 726 | O4′ | URI | 34 | 52.658 | 65.043 | −1.608 | 1.00 | 84.96 | |
| ATOM | 727 | C1′ | URI | 34 | 51.308 | 64.631 | −1.513 | 1.00 | 85.81 | |
| ATOM | 728 | N1 | URI | 34 | 51.130 | 63.311 | −2.140 | 1.00 | 86.10 | |
| ATOM | 729 | C6 | URI | 34 | 52.140 | 62.719 | −2.856 | 1.00 | 85.92 | |
| ATOM | 730 | C2 | URI | 34 | 49.894 | 62.702 | −2.023 | 1.00 | 86.59 | |
| ATOM | 731 | O2 | URI | 34 | 48.985 | 63.180 | −1.372 | 1.00 | 87.11 | |
| ATOM | 732 | N3 | URI | 34 | 49.761 | 61.507 | −2.697 | 1.00 | 87.27 | |
| ATOM | 733 | C4 | URI | 34 | 50.729 | 60.861 | −3.449 | 1.00 | 86.98 | |
| ATOM | 734 | O4 | URI | 34 | 50.485 | 59.753 | −3.952 | 1.00 | 85.30 | |
| ATOM | 735 | C5 | URI | 34 | 51.987 | 61.552 | −3.502 | 1.00 | 86.88 | |
| ATOM | 736 | C2′ | URI | 34 | 50.831 | 64.878 | −0.085 | 1.00 | 85.35 | |
| ATOM | 737 | O2′ | URI | 34 | 50.076 | 66.070 | −0.104 | 1.00 | 86.00 | |
| ATOM | 738 | C3′ | URI | 34 | 52.156 | 65.005 | 0.665 | 1.00 | 85.08 | |
| ATOM | 739 | O3′ | URI | 34 | 52.089 | 65.930 | 1.761 | 1.00 | 87.15 | |
| ATOM | 740 | P | GUA | 35 | 52.358 | 65.417 | 3.259 | 1.00 | 86.81 | |
| ATOM | 741 | O1P | GUA | 35 | 51.033 | 65.254 | 3.898 | 1.00 | 88.42 | |
| ATOM | 742 | O2P | GUA | 35 | 53.292 | 64.267 | 3.200 | 1.00 | 88.21 | |
| ATOM | 743 | O5′ | GUA | 35 | 53.106 | 66.604 | 3.992 | 1.00 | 84.49 | |
| ATOM | 744 | C5′ | GUA | 35 | 53.828 | 66.337 | 5.188 | 1.00 | 83.94 | |
| ATOM | 745 | C4′ | GUA | 35 | 54.971 | 65.416 | 4.870 | 1.00 | 81.94 | |
| ATOM | 746 | O4′ | GUA | 35 | 55.453 | 65.777 | 3.566 | 1.00 | 82.71 | |
| ATOM | 747 | C1′ | GUA | 35 | 56.852 | 65.624 | 3.534 | 1.00 | 82.73 | |
| ATOM | 748 | N9 | GUA | 35 | 57.461 | 66.837 | 2.993 | 1.00 | 81.62 | |
| ATOM | 749 | C4 | GUA | 35 | 58.789 | 67.194 | 2.992 | 1.00 | 80.29 | |
| ATOM | 750 | N3 | GUA | 35 | 59.785 | 66.571 | 3.647 | 1.00 | 80.23 | |
| ATOM | 751 | C2 | GUA | 35 | 60.971 | 67.082 | 3.339 | 1.00 | 79.25 | |
| ATOM | 752 | N2 | GUA | 35 | 62.087 | 66.580 | 3.871 | 1.00 | 79.19 | |
| ATOM | 753 | N1 | GUA | 35 | 61.154 | 68.118 | 2.476 | 1.00 | 78.99 | |
| ATOM | 754 | C6 | GUA | 35 | 60.142 | 68.772 | 1.794 | 1.00 | 79.91 | |
| ATOM | 755 | O6 | GUA | 35 | 60.419 | 69.675 | 1.003 | 1.00 | 81.27 | |
| ATOM | 756 | C5 | GUA | 35 | 58.873 | 68.250 | 2.117 | 1.00 | 80.38 | |
| ATOM | 757 | N7 | GUA | 35 | 57.613 | 68.626 | 1.672 | 1.00 | 81.32 | |
| ATOM | 758 | C8 | GUA | 35 | 56.806 | 67.783 | 2.242 | 1.00 | 81.05 | |
| ATOM | 759 | C2′ | GUA | 35 | 57.291 | 64.992 | 4.847 | 1.00 | 82.56 | |
| ATOM | 760 | O2′ | GUA | 35 | 57.197 | 63.611 | 4.577 | 1.00 | 85.76 | |
| ATOM | 761 | C3′ | GUA | 35 | 56.180 | 65.440 | 5.792 | 1.00 | 81.33 | |
| ATOM | 762 | O3′ | GUA | 35 | 56.002 | 64.420 | 6.774 | 1.00 | 79.10 | |
| ATOM | 763 | P | ADE | 36 | 56.392 | 64.687 | 8.311 | 1.00 | 76.10 | |
| ATOM | 764 | O1P | ADE | 36 | 57.292 | 65.863 | 8.400 | 1.00 | 76.28 | |
| ATOM | 765 | O2P | ADE | 36 | 55.074 | 64.698 | 9.002 | 1.00 | 75.02 | |
| ATOM | 766 | O5′ | ADE | 36 | 57.262 | 63.427 | 8.769 | 1.00 | 72.44 | |
| ATOM | 767 | C5′ | ADE | 36 | 56.689 | 62.130 | 8.937 | 1.00 | 69.73 | |
| ATOM | 768 | C4′ | ADE | 36 | 57.768 | 61.156 | 9.349 | 1.00 | 68.51 | |
| ATOM | 769 | O4′ | ADE | 36 | 58.830 | 61.193 | 8.366 | 1.00 | 69.29 | |
| ATOM | 770 | C1′ | ADE | 36 | 60.093 | 61.079 | 9.006 | 1.00 | 68.61 | |
| ATOM | 771 | N9 | ADE | 36 | 60.786 | 62.341 | 8.784 | 1.00 | 68.09 | |
| ATOM | 772 | C4 | ADE | 36 | 61.960 | 62.741 | 9.352 | 1.00 | 67.99 | |
| ATOM | 773 | N3 | ADE | 36 | 62.718 | 62.045 | 10.212 | 1.00 | 68.58 | |
| ATOM | 774 | C2 | ADE | 36 | 63.780 | 62.753 | 10.554 | 1.00 | 69.95 | |
| ATOM | 775 | N1 | ADE | 36 | 64.139 | 63.988 | 10.159 | 1.00 | 70.50 | |
| ATOM | 776 | C6 | ADE | 36 | 63.343 | 64.651 | 9.295 | 1.00 | 68.89 | |
| ATOM | 777 | N6 | ADE | 36 | 63.689 | 65.882 | 8.903 | 1.00 | 69.53 | |
| ATOM | 778 | C5 | ADE | 36 | 62.198 | 64.010 | 8.862 | 1.00 | 67.95 | |
| ATOM | 779 | N7 | ADE | 36 | 61.198 | 64.400 | 7.995 | 1.00 | 68.05 | |
| ATOM | 780 | C8 | ADE | 36 | 60.384 | 63.378 | 7.985 | 1.00 | 68.64 | |
| ATOM | 781 | C2′ | ADE | 36 | 59.830 | 60.835 | 10.492 | 1.00 | 68.54 | |
| ATOM | 782 | O2′ | ADE | 36 | 59.907 | 59.465 | 10.816 | 1.00 | 68.62 | |
| ATOM | 783 | C3′ | ADE | 36 | 58.455 | 61.472 | 10.658 | 1.00 | 68.32 | |
| ATOM | 784 | O3′ | ADE | 36 | 57.745 | 60.838 | 11.688 | 1.00 | 67.44 | |
| ATOM | 785 | P | ADE | 37 | 57.575 | 61.575 | 13.088 | 1.00 | 68.06 | |
| ATOM | 786 | O1P | ADE | 37 | 56.939 | 62.888 | 12.815 | 1.00 | 66.90 | |
| ATOM | 787 | O2P | ADE | 37 | 56.895 | 60.606 | 13.972 | 1.00 | 68.46 | |
| ATOM | 788 | O5′ | ADE | 37 | 59.070 | 61.740 | 13.622 | 1.00 | 69.76 | |
| ATOM | 789 | C5′ | ADE | 37 | 59.756 | 60.609 | 14.156 | 1.00 | 72.94 | |
| ATOM | 790 | C4′ | ADE | 37 | 61.154 | 60.964 | 14.615 | 1.00 | 74.87 | |
| ATOM | 791 | O4′ | ADE | 37 | 61.892 | 61.562 | 13.515 | 1.00 | 75.69 | |
| ATOM | 792 | C1′ | ADE | 37 | 62.810 | 62.516 | 14.016 | 1.00 | 76.23 | |
| ATOM | 793 | N9 | ADE | 37 | 62.552 | 63.812 | 13.406 | 1.00 | 77.82 | |
| ATOM | 794 | C4 | ADE | 37 | 63.425 | 64.867 | 13.432 | 1.00 | 79.71 | |
| ATOM | 795 | N3 | ADE | 37 | 64.632 | 64.905 | 14.019 | 1.00 | 81.14 | |
| ATOM | 796 | C2 | ADE | 37 | 65.213 | 66.088 | 13.817 | 1.00 | 82.14 | |
| ATOM | 797 | N1 | ADE | 37 | 64.758 | 67.157 | 13.146 | 1.00 | 82.16 | |
| ATOM | 798 | C6 | ADE | 37 | 63.536 | 67.086 | 12.576 | 1.00 | 81.25 | |
| ATOM | 799 | N6 | ADE | 37 | 63.082 | 68.154 | 11.911 | 1.00 | 81.85 | |
| ATOM | 800 | C5 | ADE | 37 | 62.815 | 65.880 | 12.720 | 1.00 | 80.27 | |
| ATOM | 801 | N7 | ADE | 37 | 61.570 | 65.474 | 12.269 | 1.00 | 79.41 | |
| ATOM | 802 | C8 | ADE | 37 | 61.464 | 64.244 | 12.706 | 1.00 | 78.94 | |
| ATOM | 803 | C2′ | ADE | 37 | 62.666 | 62.539 | 15.527 | 1.00 | 76.07 | |
| ATOM | 804 | O2′ | ADE | 37 | 63.633 | 61.667 | 16.073 | 1.00 | 75.75 | |
| ATOM | 805 | C3′ | ADE | 37 | 61.246 | 62.022 | 15.695 | 1.00 | 76.36 | |
| ATOM | 806 | O3′ | ADE | 37 | 61.031 | 61.494 | 16.991 | 1.00 | 78.92 | |
| ATOM | 807 | P | ADE | 38 | 60.280 | 62.398 | 18.086 | 1.00 | 80.29 | |
| ATOM | 808 | O1P | ADE | 38 | 58.921 | 62.691 | 17.560 | 1.00 | 80.62 | |
| ATOM | 809 | O2P | ADE | 38 | 60.431 | 61.745 | 19.400 | 1.00 | 81.52 | |
| ATOM | 810 | O5′ | ADE | 38 | 61.124 | 63.747 | 18.122 | 1.00 | 80.09 | |
| ATOM | 811 | C5′ | ADE | 38 | 62.361 | 63.800 | 18.824 | 1.00 | 81.98 | |
| ATOM | 812 | C4′ | ADE | 38 | 62.969 | 65.166 | 18.690 | 1.00 | 82.78 | |
| ATOM | 813 | O4′ | ADE | 38 | 63.264 | 65.409 | 17.291 | 1.00 | 82.57 | |
| ATOM | 814 | C1′ | ADE | 38 | 62.986 | 66.757 | 16.974 | 1.00 | 81.80 | |
| ATOM | 815 | N9 | ADE | 38 | 61.804 | 66.759 | 16.126 | 1.00 | 79.54 | |
| ATOM | 816 | C4 | ADE | 38 | 61.335 | 67.816 | 15.398 | 1.00 | 78.28 | |
| ATOM | 817 | N3 | ADE | 38 | 61.883 | 69.036 | 15.297 | 1.00 | 77.95 | |
| ATOM | 818 | C2 | ADE | 38 | 61.154 | 69.807 | 14.504 | 1.00 | 77.50 | |
| ATOM | 819 | N1 | ADE | 38 | 60.023 | 69.520 | 13.859 | 1.00 | 77.53 | |
| ATOM | 820 | C6 | ADE | 38 | 59.494 | 68.287 | 13.995 | 1.00 | 77.42 | |
| ATOM | 821 | N6 | ADE | 38 | 58.355 | 68.011 | 13.373 | 1.00 | 77.75 | |
| ATOM | 822 | C5 | ADE | 38 | 60.177 | 67.373 | 14.793 | 1.00 | 77.73 | |
| ATOM | 823 | N7 | ADE | 38 | 59.926 | 66.053 | 15.121 | 1.00 | 77.91 | |
| ATOM | 824 | C8 | ADE | 38 | 60.920 | 65.734 | 15.911 | 1.00 | 78.96 | |
| ATOM | 825 | C2′ | ADE | 38 | 62.667 | 67.461 | 18.293 | 1.00 | 83.43 | |
| ATOM | 826 | O2′ | ADE | 38 | 63.875 | 67.915 | 18.871 | 1.00 | 84.66 | |
| ATOM | 827 | C3′ | ADE | 38 | 62.066 | 66.314 | 19.082 | 1.00 | 83.66 | |
| ATOM | 828 | O3′ | ADE | 38 | 62.046 | 66.517 | 20.480 | 1.00 | 84.91 | |
| ATOM | 829 | P | CYT | 39 | 60.640 | 66.801 | 21.201 | 1.00 | 87.62 | |
| ATOM | 830 | O1P | CYT | 39 | 59.568 | 66.207 | 20.374 | 1.00 | 88.09 | |
| ATOM | 831 | O2P | CYT | 39 | 60.752 | 66.407 | 22.625 | 1.00 | 88.44 | |
| ATOM | 832 | O5′ | CYT | 39 | 60.498 | 68.381 | 21.100 | 1.00 | 86.44 | |
| ATOM | 833 | C5′ | CYT | 39 | 61.569 | 69.202 | 21.514 | 1.00 | 85.29 | |
| ATOM | 834 | C4′ | CYT | 39 | 61.421 | 70.589 | 20.957 | 1.00 | 85.25 | |
| ATOM | 835 | O4′ | CYT | 39 | 61.575 | 70.540 | 19.513 | 1.00 | 84.10 | |
| ATOM | 836 | C1′ | CYT | 39 | 60.739 | 71.516 | 18.924 | 1.00 | 84.21 | |
| ATOM | 837 | N1 | CYT | 39 | 59.716 | 70.855 | 18.114 | 1.00 | 83.58 | |
| ATOM | 838 | C6 | CYT | 39 | 59.324 | 69.579 | 18.363 | 1.00 | 83.43 | |
| ATOM | 839 | C2 | CYT | 39 | 59.136 | 71.577 | 17.088 | 1.00 | 84.14 | |
| ATOM | 840 | O2 | CYT | 39 | 59.526 | 72.728 | 16.889 | 1.00 | 86.80 | |
| ATOM | 841 | N3 | CYT | 39 | 58.174 | 71.018 | 16.337 | 1.00 | 83.32 | |
| ATOM | 842 | C4 | CYT | 39 | 57.793 | 69.777 | 16.581 | 1.00 | 83.32 | |
| ATOM | 843 | N4 | CYT | 39 | 56.847 | 69.259 | 15.811 | 1.00 | 83.16 | |
| ATOM | 844 | C5 | CYT | 39 | 58.372 | 69.006 | 17.629 | 1.00 | 83.82 | |
| ATOM | 845 | C2′ | CYT | 39 | 60.079 | 72.294 | 20.057 | 1.00 | 84.89 | |
| ATOM | 846 | O2′ | CYT | 39 | 60.929 | 73.367 | 20.402 | 1.00 | 85.96 | |
| ATOM | 847 | C3′ | CYT | 39 | 60.064 | 71.252 | 21.158 | 1.00 | 85.22 | |
| ATOM | 848 | O3′ | CYT | 39 | 59.916 | 71.851 | 22.443 | 1.00 | 86.04 | |
| ATOM | 849 | P | CYT | 40 | 58.478 | 71.831 | 23.171 | 1.00 | 85.51 | |
| ATOM | 850 | O1P | CYT | 40 | 57.835 | 70.545 | 22.831 | 1.00 | 86.98 | |
| ATOM | 851 | O2P | CYT | 40 | 58.699 | 72.174 | 24.590 | 1.00 | 84.93 | |
| ATOM | 852 | O5′ | CYT | 40 | 57.661 | 72.994 | 22.452 | 1.00 | 84.56 | |
| ATOM | 853 | C5′ | CYT | 40 | 58.127 | 74.330 | 22.513 | 1.00 | 85.44 | |
| ATOM | 854 | C4′ | CYT | 40 | 57.372 | 75.208 | 21.549 | 1.00 | 86.80 | |
| ATOM | 855 | O4′ | CYT | 40 | 57.599 | 74.745 | 20.187 | 1.00 | 86.95 | |
| ATOM | 856 | C1′ | CYT | 40 | 56.431 | 74.971 | 19.409 | 1.00 | 87.86 | |
| ATOM | 857 | N1 | CYT | 40 | 55.930 | 73.705 | 18.837 | 1.00 | 88.22 | |
| ATOM | 858 | C6 | CYT | 40 | 56.355 | 72.484 | 19.285 | 1.00 | 87.97 | |
| ATOM | 859 | C2 | CYT | 40 | 54.963 | 73.785 | 17.825 | 1.00 | 88.26 | |
| ATOM | 860 | O2 | CYT | 40 | 54.607 | 74.910 | 17.423 | 1.00 | 87.17 | |
| ATOM | 861 | N3 | CYT | 40 | 54.438 | 72.645 | 17.316 | 1.00 | 87.62 | |
| ATOM | 862 | C4 | CYT | 40 | 54.842 | 71.465 | 17.773 | 1.00 | 86.66 | |
| ATOM | 863 | N4 | CYT | 40 | 54.279 | 70.379 | 17.247 | 1.00 | 85.82 | |
| ATOM | 864 | C5 | CYT | 40 | 55.837 | 71.349 | 18.789 | 1.00 | 87.07 | |
| ATOM | 865 | C2′ | CYT | 40 | 55.389 | 75.610 | 20.327 | 1.00 | 87.81 | |
| ATOM | 866 | O2′ | CYT | 40 | 55.432 | 77.007 | 20.192 | 1.00 | 89.04 | |
| ATOM | 867 | C3′ | CYT | 40 | 55.863 | 75.155 | 21.695 | 1.00 | 87.75 | |
| ATOM | 868 | O3′ | CYT | 40 | 55.386 | 76.022 | 22.712 | 1.00 | 88.67 | |
| ATOM | 869 | P | CYT | 41 | 54.105 | 75.582 | 23.587 | 1.00 | 91.09 | |
| ATOM | 870 | O1P | CYT | 41 | 54.262 | 74.119 | 23.869 | 1.00 | 91.72 | |
| ATOM | 871 | O2P | CYT | 41 | 53.947 | 76.525 | 24.714 | 1.00 | 91.25 | |
| ATOM | 872 | O5′ | CYT | 41 | 52.845 | 75.795 | 22.632 | 1.00 | 89.14 | |
| ATOM | 873 | C5′ | CYT | 41 | 52.408 | 77.101 | 22.295 | 1.00 | 88.71 | |
| ATOM | 874 | C4′ | CYT | 41 | 51.492 | 77.048 | 21.108 | 1.00 | 88.37 | |
| ATOM | 875 | O4′ | CYT | 41 | 52.190 | 76.405 | 20.008 | 1.00 | 89.29 | |
| ATOM | 876 | C1′ | CYT | 41 | 51.273 | 75.626 | 19.256 | 1.00 | 89.60 | |
| ATOM | 877 | N1 | CYT | 41 | 51.646 | 74.205 | 19.348 | 1.00 | 91.04 | |
| ATOM | 878 | C6 | CYT | 41 | 52.561 | 73.747 | 20.259 | 1.00 | 91.58 | |
| ATOM | 879 | C2 | CYT | 41 | 50.996 | 73.321 | 18.502 | 1.00 | 91.43 | |
| ATOM | 880 | O2 | CYT | 41 | 50.212 | 73.784 | 17.665 | 1.00 | 92.37 | |
| ATOM | 881 | N3 | CYT | 41 | 51.228 | 71.995 | 18.614 | 1.00 | 91.74 | |
| ATOM | 882 | C4 | CYT | 41 | 52.088 | 71.545 | 19.523 | 1.00 | 92.37 | |
| ATOM | 883 | N4 | CYT | 41 | 52.258 | 70.220 | 19.610 | 1.00 | 93.52 | |
| ATOM | 884 | C5 | CYT | 41 | 52.808 | 72.431 | 20.384 | 1.00 | 92.23 | |
| ATOM | 885 | C2′ | CYT | 41 | 49.890 | 75.820 | 19.880 | 1.00 | 88.60 | |
| ATOM | 886 | O2′ | CYT | 41 | 49.181 | 76.836 | 19.219 | 1.00 | 88.99 | |
| ATOM | 887 | C3′ | CYT | 41 | 50.252 | 76.197 | 21.305 | 1.00 | 87.64 | |
| ATOM | 888 | O3′ | CYT | 41 | 49.207 | 76.938 | 21.898 | 1.00 | 85.91 | |
| ATOM | 889 | P | GUA | 42 | 48.291 | 76.244 | 23.009 | 1.00 | 86.55 | |
| ATOM | 890 | O1P | GUA | 42 | 49.233 | 75.429 | 23.810 | 1.00 | 87.91 | |
| ATOM | 891 | O2P | GUA | 42 | 47.411 | 77.221 | 23.689 | 1.00 | 88.51 | |
| ATOM | 892 | O5′ | GUA | 42 | 47.322 | 75.299 | 22.181 | 1.00 | 85.77 | |
| ATOM | 893 | C5′ | GUA | 42 | 46.572 | 75.815 | 21.102 | 1.00 | 83.65 | |
| ATOM | 894 | C4′ | GUA | 42 | 46.081 | 74.691 | 20.219 | 1.00 | 83.03 | |
| ATOM | 895 | O4′ | GUA | 42 | 47.221 | 74.023 | 19.599 | 1.00 | 82.47 | |
| ATOM | 896 | C1′ | GUA | 42 | 46.940 | 72.642 | 19.454 | 1.00 | 82.41 | |
| ATOM | 897 | N9 | GUA | 42 | 47.944 | 71.866 | 20.181 | 1.00 | 83.09 | |
| ATOM | 898 | C4 | GUA | 42 | 47.988 | 70.494 | 20.284 | 1.00 | 83.04 | |
| ATOM | 899 | N3 | GUA | 42 | 47.132 | 69.627 | 19.703 | 1.00 | 83.00 | |
| ATOM | 900 | C2 | GUA | 42 | 47.418 | 68.371 | 20.008 | 1.00 | 83.95 | |
| ATOM | 901 | N2 | OUA | 42 | 46.672 | 67.373 | 19.513 | 1.00 | 84.35 | |
| ATOM | 902 | N1 | OUA | 42 | 48.457 | 67.994 | 20.824 | 1.00 | 83.49 | |
| ATOM | 903 | C6 | GUA | 42 | 49.349 | 68.864 | 21.432 | 1.00 | 83.67 | |
| ATOM | 904 | O6 | GUA | 42 | 50.245 | 68.416 | 22.156 | 1.00 | 84.51 | |
| ATOM | 905 | C5 | GUA | 42 | 49.061 | 70.223 | 21.104 | 1.00 | 83.28 | |
| ATOM | 906 | N7 | GUA | 42 | 49.697 | 71.396 | 21.487 | 1.00 | 83.24 | |
| ATOM | 907 | C8 | GUA | 42 | 49.005 | 72.343 | 20.911 | 1.00 | 82.90 | |
| ATOM | 908 | C2′ | GUA | 42 | 45.523 | 72.413 | 19.980 | 1.00 | 81.95 | |
| ATOM | 909 | O2′ | GUA | 42 | 44.613 | 72.545 | 18.912 | 1.00 | 82.94 | |
| ATOM | 910 | C3′ | GUA | 42 | 45.366 | 73.570 | 20.950 | 1.00 | 81.55 | |
| ATOM | 911 | O3′ | GUA | 42 | 44.003 | 73.863 | 21.197 | 1.00 | 79.48 | |
| ATOM | 912 | P | GUA | 43 | 43.311 | 73.294 | 22.526 | 1.00 | 79.57 | |
| ATOM | 913 | O1P | GUA | 43 | 41.923 | 73.802 | 22.627 | 1.00 | 81.31 | |
| ATOM | 914 | O2P | GUA | 43 | 44.271 | 73.553 | 23.625 | 1.00 | 81.40 | |
| ATOM | 915 | O5′ | GUA | 43 | 43.199 | 71.717 | 22.304 | 1.00 | 78.96 | |
| ATOM | 916 | C5′ | GUA | 43 | 42.378 | 71.192 | 21.269 | 1.00 | 75.51 | |
| ATOM | 917 | C4′ | GUA | 43 | 42.530 | 69.691 | 21.152 | 1.00 | 72.80 | |
| ATOM | 918 | O4′ | GUA | 43 | 43.922 | 69.367 | 20.875 | 1.00 | 71.82 | |
| ATOM | 919 | C1′ | GUA | 43 | 44.236 | 68.119 | 21.463 | 1.00 | 71.99 | |
| ATOM | 920 | N9 | GUA | 43 | 45.387 | 68.259 | 22.340 | 1.00 | 73.13 | |
| ATOM | 921 | C4 | GUA | 43 | 46.110 | 67.227 | 22.889 | 1.00 | 74.60 | |
| ATOM | 922 | N3 | GUA | 43 | 45.872 | 65.913 | 22.714 | 1.00 | 75.27 | |
| ATOM | 923 | C2 | GUA | 43 | 46.725 | 65.163 | 23.384 | 1.00 | 75.13 | |
| ATOM | 924 | N2 | GUA | 43 | 46.613 | 63.851 | 23.342 | 1.00 | 74.63 | |
| ATOM | 925 | N1 | GUA | 43 | 47.743 | 65.659 | 24.150 | 1.00 | 75.79 | |
| ATOM | 926 | C6 | GUA | 43 | 48.013 | 67.009 | 24.338 | 1.00 | 75.56 | |
| ATOM | 927 | O6 | GUA | 43 | 48.963 | 67.351 | 25.045 | 1.00 | 76.20 | |
| ATOM | 928 | C5 | GUA | 43 | 47.094 | 67.826 | 23.638 | 1.00 | 75.10 | |
| ATOM | 929 | N7 | GUA | 43 | 46.999 | 69.205 | 23.557 | 1.00 | 75.83 | |
| ATOM | 930 | C8 | GUA | 43 | 45.970 | 69.415 | 22.777 | 1.00 | 74.59 | |
| ATOM | 931 | C2′ | GUA | 43 | 42.987 | 67.605 | 22.168 | 1.00 | 71.18 | |
| ATOM | 932 | O2′ | GUA | 43 | 42.315 | 66.733 | 21.288 | 1.00 | 69.49 | |
| ATOM | 933 | C3′ | GUA | 43 | 42.214 | 68.893 | 22.409 | 1.00 | 70.80 | |
| ATOM | 934 | O3′ | GUA | 43 | 40.825 | 68.605 | 22.511 | 1.00 | 68.71 | |
| ATOM | 935 | P | CYT | 44 | 40.255 | 67.900 | 23.839 | 1.00 | 68.64 | |
| ATOM | 936 | O1P | CYT | 44 | 40.877 | 68.613 | 24.984 | 1.00 | 70.66 | |
| ATOM | 937 | O2P | CYT | 44 | 38.783 | 67.761 | 23.784 | 1.00 | 68.94 | |
| ATOM | 938 | O5′ | CYT | 44 | 40.878 | 66.434 | 23.823 | 1.00 | 68.91 | |
| ATOM | 939 | C5′ | CYT | 44 | 40.511 | 65.476 | 22.826 | 1.00 | 66.80 | |
| ATOM | 940 | C4′ | CYT | 44 | 41.134 | 64.139 | 23.152 | 1.00 | 64.15 | |
| ATOM | 941 | O4′ | CYT | 44 | 42.574 | 64.264 | 23.119 | 1.00 | 62.13 | |
| ATOM | 942 | C1′ | CYT | 44 | 43.140 | 63.469 | 24.141 | 1.00 | 60.43 | |
| ATOM | 943 | N1 | CYT | 44 | 43.895 | 64.367 | 25.013 | 1.00 | 57.90 | |
| ATOM | 944 | C6 | CYT | 44 | 43.864 | 65.707 | 24.802 | 1.00 | 56.91 | |
| ATOM | 945 | C2 | CYT | 44 | 44.669 | 63.833 | 26.024 | 1.00 | 56.80 | |
| ATOM | 946 | O2 | CYT | 44 | 44.620 | 62.625 | 26.233 | 1.00 | 55.50 | |
| ATOM | 947 | N3 | CYT | 44 | 45.445 | 64.643 | 26.755 | 1.00 | 56.80 | |
| ATOM | 948 | C4 | CYT | 44 | 45.447 | 65.944 | 26.511 | 1.00 | 57.82 | |
| ATOM | 949 | N4 | CYT | 44 | 46.254 | 66.709 | 27.224 | 1.00 | 58.48 | |
| ATOM | 950 | C5 | CYT | 44 | 44.622 | 66.522 | 25.515 | 1.00 | 57.76 | |
| ATOM | 951 | C2′ | CYT | 44 | 41.998 | 62.732 | 24.841 | 1.00 | 61.48 | |
| ATOM | 952 | O2′ | CYT | 44 | 41.760 | 61.468 | 24.271 | 1.00 | 61.47 | |
| ATOM | 953 | C3′ | CYT | 44 | 40.831 | 63.644 | 24.551 | 1.00 | 62.54 | |
| ATOM | 954 | O3′ | CYT | 44 | 39.684 | 62.836 | 24.529 | 1.00 | 63.79 | |
| ATOM | 955 | P | ADE | 45 | 39.014 | 62.409 | 25.914 | 1.00 | 66.89 | |
| ATOM | 956 | O1P | ADE | 45 | 38.560 | 63.712 | 26.464 | 1.00 | 67.71 | |
| ATOM | 957 | O2P | ADE | 45 | 38.030 | 61.286 | 25.740 | 1.00 | 65.87 | |
| ATOM | 958 | O5′ | ADE | 45 | 40.238 | 61.881 | 26.789 | 1.00 | 63.98 | |
| ATOM | 959 | C5′ | ADE | 45 | 40.614 | 60.511 | 26.768 | 1.00 | 61.57 | |
| ATOM | 960 | C4′ | ADE | 45 | 41.581 | 60.247 | 27.876 | 1.00 | 60.53 | |
| ATOM | 961 | O4′ | ADE | 45 | 42.723 | 61.117 | 27.717 | 1.00 | 58.99 | |
| ATOM | 962 | C1′ | ADE | 45 | 43.162 | 61.547 | 28.982 | 1.00 | 57.65 | |
| ATOM | 963 | N9 | ADE | 45 | 43.051 | 62.999 | 28.965 | 1.00 | 57.49 | |
| ATOM | 964 | C4 | ADE | 45 | 43.876 | 63.896 | 29.582 | 1.00 | 56.54 | |
| ATOM | 965 | N3 | ADE | 45 | 44.931 | 63.624 | 30.368 | 1.00 | 55.90 | |
| ATOM | 966 | C2 | ADE | 45 | 45.513 | 64.733 | 30.754 | 1.00 | 56.23 | |
| ATOM | 967 | N1 | ADE | 45 | 45.180 | 65.997 | 30.483 | 1.00 | 57.79 | |
| ATOM | 968 | C6 | ADE | 45 | 44.101 | 66.229 | 29.700 | 1.00 | 57.33 | |
| ATOM | 969 | N6 | ADE | 45 | 43.739 | 67.484 | 29.449 | 1.00 | 57.00 | |
| ATOM | 970 | C5 | ADE | 45 | 43.419 | 65.136 | 29.209 | 1.00 | 56.21 | |
| ATOM | 971 | N7 | ADE | 45 | 42.316 | 65.030 | 28.387 | 1.00 | 56.07 | |
| ATOM | 972 | C8 | ADE | 45 | 42.128 | 63.745 | 28.285 | 1.00 | 56.50 | |
| ATOM | 973 | C2′ | ADE | 45 | 42.308 | 60.837 | 30.039 | 1.00 | 58.16 | |
| ATOM | 974 | O2′ | ADE | 45 | 42.920 | 59.643 | 30.428 | 1.00 | 59.75 | |
| ATOM | 975 | C3′ | ADE | 45 | 41.042 | 60.526 | 29.264 | 1.00 | 59.89 | |
| ATOM | 976 | O3′ | ADE | 45 | 40.528 | 59.285 | 29.711 | 1.00 | 62.06 | |
| ATOM | 977 | P | ADE | 46 | 39.615 | 59.213 | 31.028 | 1.00 | 66.31 | |
| ATOM | 978 | O1P | ADE | 46 | 38.691 | 60.370 | 30.946 | 1.00 | 65.21 | |
| ATOM | 979 | O2P | ADE | 46 | 39.082 | 57.826 | 31.179 | 1.00 | 63.84 | |
| ATOM | 980 | O5′ | ADE | 46 | 40.600 | 59.481 | 32.250 | 1.00 | 67.39 | |
| ATOM | 981 | C5′ | ADE | 46 | 41.166 | 58.419 | 33.024 | 1.00 | 66.57 | |
| ATOM | 982 | C4′ | ADE | 46 | 42.118 | 59.027 | 33.994 | 1.00 | 66.83 | |
| ATOM | 983 | O4′ | ADE | 46 | 42.721 | 60.115 | 33.283 | 1.00 | 67.97 | |
| ATOM | 984 | C1′ | ADE | 46 | 42.902 | 61.202 | 34.150 | 1.00 | 66.90 | |
| ATOM | 985 | N9 | ADE | 46 | 42.144 | 62.278 | 33.562 | 1.00 | 63.68 | |
| ATOM | 986 | C4 | ADE | 46 | 42.591 | 63.550 | 33.366 | 1.00 | 63.94 | |
| ATOM | 987 | N3 | ADE | 46 | 43.751 | 64.068 | 33.779 | 1.00 | 64.11 | |
| ATOM | 988 | C2 | ADE | 46 | 43.876 | 65.317 | 33.351 | 1.00 | 63.21 | |
| ATOM | 989 | N1 | ADE | 46 | 43.045 | 66.037 | 32.616 | 1.00 | 63.40 | |
| ATOM | 990 | C6 | ADE | 46 | 41.888 | 65.480 | 32.222 | 1.00 | 63.37 | |
| ATOM | 991 | N6 | ADE | 46 | 41.058 | 66.203 | 31.479 | 1.00 | 65.98 | |
| ATOM | 992 | C5 | ADE | 46 | 41.629 | 64.176 | 32.610 | 1.00 | 63.27 | |
| ATOM | 993 | N7 | ADE | 46 | 40.559 | 63.325 | 32.377 | 1.00 | 64.01 | |
| ATOM | 994 | C8 | ADE | 46 | 40.908 | 62.217 | 32.987 | 1.00 | 63.56 | |
| ATOM | 995 | C2′ | ADE | 46 | 42.492 | 60.746 | 35.552 | 1.00 | 69.88 | |
| ATOM | 996 | O2′ | ADE | 46 | 43.560 | 60.131 | 36.270 | 1.00 | 73.70 | |
| ATOM | 997 | C3′ | ADE | 46 | 41.508 | 59.649 | 35.239 | 1.00 | 68.18 | |
| ATOM | 998 | O3′ | ADE | 46 | 41.708 | 58.864 | 36.418 | 1.00 | 68.73 | |
| ATOM | 999 | P | CYT | 47 | 42.593 | 57.526 | 36.381 | 1.00 | 68.81 | |
| ATOM | 1000 | O1P | CYT | 47 | 42.991 | 57.200 | 35.000 | 1.00 | 73.24 | |
| ATOM | 1001 | O2P | CYT | 47 | 41.866 | 56.507 | 37.171 | 1.00 | 71.86 | |
| ATOM | 1002 | O5′ | CYT | 47 | 43.938 | 57.788 | 37.191 | 1.00 | 66.01 | |
| ATOM | 1003 | C5′ | CYT | 47 | 44.681 | 56.630 | 37.627 | 1.00 | 63.86 | |
| ATOM | 1004 | C4′ | CYT | 47 | 46.149 | 56.933 | 37.891 | 1.00 | 62.26 | |
| ATOM | 1005 | O4′ | CYT | 47 | 46.832 | 57.320 | 36.671 | 1.00 | 59.06 | |
| ATOM | 1006 | C1′ | CYT | 47 | 47.812 | 58.288 | 36.974 | 1.00 | 57.28 | |
| ATOM | 1007 | N1 | CYT | 47 | 47.478 | 59.535 | 36.307 | 1.00 | 53.11 | |
| ATOM | 1008 | C6 | CYT | 47 | 46.302 | 59.710 | 35.657 | 1.00 | 52.47 | |
| ATOM | 1009 | C2 | CYT | 47 | 48.408 | 60.533 | 36.346 | 1.00 | 52.47 | |
| ATOM | 1010 | O2 | CYT | 47 | 49.457 | 60.316 | 36.970 | 1.00 | 52.47 | |
| ATOM | 1011 | N3 | CYT | 47 | 48.166 | 61.704 | 35.714 | 1.00 | 51.60 | |
| ATOM | 1012 | C4 | CYT | 47 | 47.020 | 61.869 | 35.064 | 1.00 | 52.29 | |
| ATOM | 1013 | N4 | CYT | 47 | 46.810 | 63.020 | 34.436 | 1.00 | 52.68 | |
| ATOM | 1014 | C5 | CYT | 47 | 46.035 | 60.856 | 35.024 | 1.00 | 52.76 | |
| ATOM | 1015 | C2′ | CYT | 47 | 47.832 | 58.483 | 38.482 | 1.00 | 60.42 | |
| ATOM | 1016 | O2′ | CYT | 47 | 48.807 | 57.640 | 39.054 | 1.00 | 61.21 | |
| ATOM | 1017 | C3′ | CYT | 47 | 46.420 | 58.075 | 38.850 | 1.00 | 63.01 | |
| ATOM | 1018 | O3′ | CYT | 47 | 46.354 | 57.610 | 40.183 | 1.00 | 67.44 | |
| ATOM | 1019 | P | CYT | 48 | 45.836 | 58.598 | 41.338 | 1.00 | 70.64 | |
| ATOM | 1020 | O1P | CYT | 48 | 44.758 | 59.475 | 40.771 | 1.00 | 68.91 | |
| ATOM | 1021 | O2P | CYT | 48 | 45.580 | 57.730 | 42.529 | 1.00 | 67.67 | |
| ATOM | 1022 | O5′ | CYT | 48 | 47.079 | 59.544 | 41.636 | 1.00 | 68.89 | |
| ATOM | 1023 | C5′ | CYT | 48 | 48.295 | 59.005 | 42.124 | 1.00 | 67.09 | |
| ATOM | 1024 | C4′ | CYT | 48 | 49.298 | 60.104 | 42.249 | 1.00 | 66.18 | |
| ATOM | 1025 | O4′ | CYT | 48 | 49.580 | 60.632 | 40.934 | 1.00 | 63.10 | |
| ATOM | 1026 | C1′ | CYT | 48 | 49.794 | 62.024 | 41.022 | 1.00 | 60.73 | |
| ATOM | 1027 | N1 | CYT | 48 | 48.827 | 62.688 | 40.166 | 1.00 | 55.29 | |
| ATOM | 1028 | C6 | CYT | 48 | 47.704 | 62.053 | 39.734 | 1.00 | 54.66 | |
| ATOM | 1029 | C2 | CYT | 48 | 49.081 | 63.987 | 39.796 | 1.00 | 55.37 | |
| ATOM | 1030 | O2 | CYT | 48 | 50.105 | 64.541 | 40.239 | 1.00 | 55.83 | |
| ATOM | 1031 | N3 | CYT | 48 | 48.220 | 64.627 | 38.976 | 1.00 | 54.10 | |
| ATOM | 1032 | C4 | CYT | 48 | 47.133 | 64.000 | 38.542 | 1.00 | 52.48 | |
| ATOM | 1033 | N4 | CYT | 48 | 46.329 | 64.661 | 37.724 | 1.00 | 52.66 | |
| ATOM | 1034 | C5 | CYT | 48 | 46.834 | 62.671 | 38.925 | 1.00 | 53.19 | |
| ATOM | 1035 | C2′ | CYT | 48 | 49.611 | 62.432 | 42.477 | 1.00 | 63.65 | |
| ATOM | 1036 | O2′ | CYT | 48 | 50.856 | 62.453 | 43.138 | 1.00 | 62.87 | |
| ATOM | 1037 | C3′ | CYT | 48 | 48.733 | 61.305 | 42.970 | 1.00 | 66.98 | |
| ATOM | 1038 | O3′ | CYT | 48 | 48.794 | 61.136 | 44.361 | 1.00 | 73.12 | |
| ATOM | 1039 | P | ADE | 49 | 47.598 | 61.690 | 45.251 | 1.00 | 75.65 | |
| ATOM | 1040 | O1P | ADE | 49 | 46.365 | 61.403 | 44.480 | 1.00 | 77.90 | |
| ATOM | 1041 | O2P | ADE | 49 | 47.779 | 61.089 | 46.598 | 1.00 | 77.55 | |
| ATOM | 1042 | O5′ | ADE | 49 | 47.879 | 63.257 | 45.283 | 1.00 | 74.70 | |
| ATOM | 1043 | C5′ | ADE | 49 | 48.993 | 63.739 | 46.013 | 1.00 | 75.40 | |
| ATOM | 1044 | C4′ | ADE | 49 | 49.273 | 65.179 | 45.679 | 1.00 | 76.51 | |
| ATOM | 1045 | O4′ | ADE | 49 | 49.405 | 65.301 | 44.239 | 1.00 | 75.68 | |
| ATOM | 1046 | C1′ | ADE | 49 | 48.946 | 66.576 | 43.819 | 1.00 | 74.51 | |
| ATOM | 1047 | N9 | ADE | 49 | 47.844 | 66.405 | 42.889 | 1.00 | 72.07 | |
| ATOM | 1048 | C4 | ADE | 49 | 47.338 | 67.391 | 42.089 | 1.00 | 70.18 | |
| ATOM | 1049 | N3 | ADE | 49 | 47.774 | 68.651 | 41.989 | 1.00 | 70.59 | |
| ATOM | 1050 | C2 | ADE | 49 | 47.019 | 69.343 | 41.130 | 1.00 | 69.94 | |
| ATOM | 1051 | N1 | ADE | 49 | 45.963 | 68.939 | 40.423 | 1.00 | 69.56 | |
| ATOM | 1052 | C6 | ADE | 49 | 45.561 | 67.655 | 40.547 | 1.00 | 69.78 | |
| ATOM | 1053 | N6 | ADE | 49 | 44.520 | 67.237 | 39.833 | 1.00 | 70.85 | |
| ATOM | 1054 | C5 | ADE | 49 | 46.271 | 66.829 | 41.421 | 1.00 | 69.83 | |
| ATOM | 1055 | N7 | ADE | 49 | 46.116 | 65.496 | 41.774 | 1.00 | 71.24 | |
| ATOM | 1056 | C8 | ADE | 49 | 47.082 | 65.291 | 42.645 | 1.00 | 72.60 | |
| ATOM | 1057 | C2′ | ADE | 49 | 48.481 | 67.317 | 45.062 | 1.00 | 77.53 | |
| ATOM | 1058 | O2′ | ADE | 49 | 49.566 | 68.095 | 45.519 | 1.00 | 79.23 | |
| ATOM | 1059 | C3′ | ADE | 49 | 48.150 | 66.154 | 45.990 | 1.00 | 78.90 | |
| ATOM | 1060 | O3′ | ADE | 49 | 48.083 | 66.512 | 47.372 | 1.00 | 82.77 | |
| ATOM | 1061 | P | GUA | 50 | 46.650 | 66.553 | 48.123 | 1.00 | 85.23 | |
| ATOM | 1062 | O1P | GUA | 50 | 45.852 | 65.364 | 47.697 | 1.00 | 85.76 | |
| ATOM | 1063 | O2P | GUA | 50 | 46.903 | 66.767 | 49.572 | 1.00 | 85.84 | |
| ATOM | 1064 | O5′ | GUA | 50 | 45.955 | 67.859 | 47.529 | 1.00 | 84.97 | |
| ATOM | 1065 | C5′ | GUA | 50 | 46.510 | 69.140 | 47.766 | 1.00 | 85.81 | |
| ATOM | 1066 | C4′ | GUA | 50 | 45.665 | 70.210 | 47.114 | 1.00 | 88.01 | |
| ATOM | 1067 | O4′ | GUA | 50 | 45.897 | 70.193 | 45.674 | 1.00 | 86.37 | |
| ATOM | 1068 | C1′ | GUA | 50 | 44.690 | 70.511 | 45.000 | 1.00 | 84.98 | |
| ATOM | 1069 | N9 | GUA | 50 | 44.230 | 69.333 | 44.269 | 1.00 | 80.72 | |
| ATOM | 1070 | C4 | GUA | 50 | 43.404 | 69.351 | 43.181 | 1.00 | 77.06 | |
| ATOM | 1071 | N3 | GUA | 50 | 42.983 | 70.454 | 42.541 | 1.00 | 75.75 | |
| ATOM | 1072 | C2 | GUA | 50 | 42.104 | 70.175 | 41.606 | 1.00 | 75.87 | |
| ATOM | 1073 | N2 | GUA | 50 | 41.559 | 71.166 | 40.884 | 1.00 | 75.39 | |
| ATOM | 1074 | N1 | GUA | 50 | 41.684 | 68.905 | 41.313 | 1.00 | 76.27 | |
| ATOM | 1075 | C6 | GUA | 50 | 42.115 | 67.747 | 41.958 | 1.00 | 76.59 | |
| ATOM | 1076 | O6 | GUA | 50 | 41.647 | 66.642 | 41.632 | 1.00 | 75.60 | |
| ATOM | 1077 | C5 | GUA | 50 | 43.061 | 68.039 | 42.961 | 1.00 | 76.74 | |
| ATOM | 1078 | N7 | GUA | 50 | 43.737 | 67.196 | 43.835 | 1.00 | 78.03 | |
| ATOM | 1079 | C8 | GUA | 50 | 44.439 | 68.007 | 44.580 | 1.00 | 79.39 | |
| ATOM | 1080 | C2′ | GUA | 50 | 43.649 | 70.870 | 46.075 | 1.00 | 87.27 | |
| ATOM | 1081 | O2′ | GUA | 50 | 43.653 | 72.255 | 46.365 | 1.00 | 87.07 | |
| ATOM | 1082 | C3′ | GUA | 50 | 44.152 | 70.060 | 47.258 | 1.00 | 88.85 | |
| ATOM | 1083 | O3′ | GUA | 50 | 43.633 | 70.550 | 48.491 | 1.00 | 93.34 | |
| ATOM | 1084 | P | ADE | 51 | 42.629 | 69.621 | 49.361 | 1.00 | 97.97 | |
| ATOM | 1085 | O1P | ADE | 51 | 43.176 | 68.230 | 49.407 | 1.00 | 97.32 | |
| ATOM | 1086 | O2P | ADE | 51 | 42.329 | 70.327 | 50.639 | 1.00 | 97.70 | |
| ATOM | 1087 | O5′ | ADE | 51 | 41.278 | 69.550 | 48.510 | 1.00 | 97.11 | |
| ATOM | 1088 | C5′ | ADE | 51 | 40.510 | 68.355 | 48.515 | 1.00 | 96.61 | |
| ATOM | 1089 | C4′ | ADE | 51 | 39.028 | 68.650 | 48.497 | 1.00 | 96.63 | |
| ATOM | 1090 | O4′ | ADE | 51 | 38.780 | 69.801 | 49.359 | 1.00 | 95.02 | |
| ATOM | 1091 | C1′ | ADE | 51 | 38.403 | 70.939 | 48.591 | 1.00 | 93.36 | |
| ATOM | 1092 | N9 | ADE | 51 | 39.400 | 71.986 | 48.740 | 1.00 | 89.82 | |
| ATOM | 1093 | C4 | ADE | 51 | 39.221 | 73.260 | 48.282 | 1.00 | 87.57 | |
| ATOM | 1094 | N3 | ADE | 51 | 38.133 | 73.751 | 47.682 | 1.00 | 86.86 | |
| ATOM | 1095 | C2 | ADE | 51 | 38.327 | 75.016 | 47.346 | 1.00 | 86.96 | |
| ATOM | 1096 | N1 | ADE | 51 | 39.395 | 75.786 | 47.544 | 1.00 | 86.26 | |
| ATOM | 1097 | C6 | ADE | 51 | 40.460 | 75.261 | 48.177 | 1.00 | 86.03 | |
| ATOM | 1098 | N6 | ADE | 51 | 41.502 | 76.045 | 48.434 | 1.00 | 85.56 | |
| ATOM | 1099 | C5 | ADE | 51 | 40.394 | 73.924 | 48.550 | 1.00 | 86.81 | |
| ATOM | 1100 | N7 | ADE | 51 | 41.302 | 73.083 | 49.169 | 1.00 | 87.54 | |
| ATOM | 1101 | C8 | ADE | 51 | 40.654 | 71.952 | 49.272 | 1.00 | 88.45 | |
| ATOM | 1102 | C2′ | ADE | 51 | 38.356 | 70.579 | 47.113 | 1.00 | 96.14 | |
| ATOM | 1103 | O2′ | ADE | 51 | 37.152 | 71.070 | 46.550 | 1.00 | 96.69 | |
| ATOM | 1104 | C3′ | ADE | 51 | 38.458 | 69.067 | 47.147 | 1.00 | 97.18 | |
| ATOM | 1105 | O3′ | ADE | 51 | 38.329 | 68.205 | 46.023 | 1.00 | 99.75 | |
| ATOM | 1106 | P | ADE | 52 | 39.320 | 68.396 | 44.761 | 1.00 | 100.35 | |
| ATOM | 1107 | O1P | ADE | 52 | 40.277 | 69.494 | 45.086 | 1.00 | 99.62 | |
| ATOM | 1108 | O2P | ADE | 52 | 39.844 | 67.045 | 44.417 | 1.00 | 99.86 | |
| ATOM | 1109 | O5′ | ADE | 52 | 38.357 | 68.907 | 43.597 | 1.00 | 100.84 | |
| ATOM | 1110 | C5′ | ADE | 52 | 38.859 | 69.666 | 42.494 | 1.00 | 102.85 | |
| ATOM | 1111 | C4′ | ADE | 52 | 38.150 | 70.995 | 42.428 | 1.00 | 104.05 | |
| ATOM | 1112 | O4′ | ADE | 52 | 38.065 | 71.525 | 43.772 | 1.00 | 102.84 | |
| ATOM | 1113 | C1′ | ADE | 52 | 38.253 | 72.919 | 43.741 | 1.00 | 101.65 | |
| ATOM | 1114 | N9 | ADE | 52 | 39.535 | 73.163 | 44.380 | 1.00 | 98.65 | |
| ATOM | 1115 | C4 | ADE | 52 | 40.204 | 74.352 | 44.488 | 1.00 | 97.34 | |
| ATOM | 1116 | N3 | ADE | 52 | 39.809 | 75.554 | 44.039 | 1.00 | 96.49 | |
| ATOM | 1117 | C2 | ADE | 52 | 40.723 | 76.476 | 44.314 | 1.00 | 95.84 | |
| ATOM | 1118 | N1 | ADE | 52 | 41.898 | 76.344 | 44.938 | 1.00 | 95.33 | |
| ATOM | 1119 | C6 | ADE | 52 | 42.262 | 75.121 | 45.373 | 1.00 | 95.42 | |
| ATOM | 1120 | N6 | ADE | 52 | 43.434 | 74.985 | 45.988 | 1.00 | 95.01 | |
| ATOM | 1121 | C5 | ADE | 52 | 41.380 | 74.059 | 45.147 | 1.00 | 96.50 | |
| ATOM | 1122 | N7 | ADE | 52 | 41.444 | 72.709 | 45.458 | 1.00 | 96.88 | |
| ATOM | 1123 | C8 | ADE | 52 | 40.327 | 72.227 | 44.986 | 1.00 | 97.13 | |
| ATOM | 1124 | C2′ | ADE | 52 | 38.218 | 73.346 | 42.269 | 1.00 | 103.84 | |
| ATOM | 1125 | O2′ | ADE | 52 | 36.882 | 73.625 | 41.924 | 1.00 | 105.50 | |
| ATOM | 1126 | C3′ | ADE | 52 | 38.760 | 72.101 | 41.571 | 1.00 | 105.31 | |
| ATOM | 1127 | O3′ | ADE | 52 | 38.309 | 71.995 | 40.204 | 1.00 | 108.14 | |
| ATOM | 1128 | P | ADE | 53 | 38.813 | 73.061 | 39.083 | 1.00 | 110.54 | |
| ATOM | 1129 | O1P | ADE | 53 | 40.294 | 73.062 | 39.046 | 1.00 | 111.14 | |
| ATOM | 1130 | O2P | ADE | 53 | 38.056 | 72.862 | 37.814 | 1.00 | 110.62 | |
| ATOM | 1131 | O5′ | ADE | 53 | 38.371 | 74.478 | 39.648 | 1.00 | 109.49 | |
| ATOM | 1132 | C5′ | ADE | 53 | 37.921 | 75.495 | 38.770 | 1.00 | 109.51 | |
| ATOM | 1133 | C4′ | ADE | 53 | 38.946 | 76.585 | 38.708 | 1.00 | 109.35 | |
| ATOM | 1134 | O4′ | ADE | 53 | 39.349 | 76.877 | 40.065 | 1.00 | 109.96 | |
| ATOM | 1135 | C1′ | ADE | 53 | 40.734 | 77.159 | 40.102 | 1.00 | 110.06 | |
| ATOM | 1136 | N9 | ADE | 53 | 41.369 | 76.060 | 40.815 | 1.00 | 110.37 | |
| ATOM | 1137 | C4 | ADE | 53 | 42.503 | 76.118 | 41.575 | 1.00 | 110.78 | |
| ATOM | 1138 | N3 | ADE | 53 | 43.229 | 77.209 | 41.866 | 1.00 | 111.45 | |
| ATOM | 1139 | C2 | ADE | 53 | 44.278 | 76.882 | 42.612 | 1.00 | 111.91 | |
| ATOM | 1140 | N1 | ADE | 53 | 44.653 | 75.675 | 43.063 | 1.00 | 112.16 | |
| ATOM | 1141 | C6 | ADE | 53 | 43.895 | 74.599 | 42.749 | 1.00 | 111.85 | |
| ATOM | 1142 | N6 | ADE | 53 | 44.267 | 73.392 | 43.195 | 1.00 | 112.22 | |
| ATOM | 1143 | C5 | ADE | 53 | 42.756 | 74.817 | 41.968 | 1.00 | 111.22 | |
| ATOM | 1144 | N7 | ADE | 53 | 41.779 | 73.960 | 41.488 | 1.00 | 110.82 | |
| ATOM | 1145 | C8 | ADE | 53 | 40.978 | 74.749 | 40.823 | 1.00 | 110.65 | |
| ATOM | 1146 | C2′ | ADE | 53 | 41.222 | 77.187 | 38.653 | 1.00 | 109.58 | |
| ATOM | 1147 | O2′ | ADE | 53 | 41.137 | 78.493 | 38.120 | 1.00 | 109.80 | |
| ATOM | 1148 | C3′ | ADE | 53 | 40.239 | 76.235 | 37.997 | 1.00 | 109.23 | |
| ATOM | 1149 | O3′ | ADE | 53 | 40.129 | 76.528 | 36.615 | 1.00 | 108.59 | |
| ATOM | 1150 | P | URI | 54 | 40.623 | 75.449 | 35.539 | 1.00 | 108.15 | |
| ATOM | 1151 | O1P | URI | 54 | 39.807 | 74.218 | 35.767 | 1.00 | 107.91 | |
| ATOM | 1152 | O2P | URI | 54 | 40.603 | 76.113 | 34.210 | 1.00 | 107.80 | |
| ATOM | 1153 | O5′ | URI | 54 | 42.143 | 75.177 | 35.945 | 1.00 | 105.25 | |
| ATOM | 1154 | C5′ | URI | 54 | 43.077 | 76.252 | 35.978 | 1.00 | 101.05 | |
| ATOM | 1155 | C4′ | URI | 54 | 44.203 | 75.954 | 36.942 | 1.00 | 98.65 | |
| ATOM | 1156 | O4′ | URI | 54 | 43.682 | 75.172 | 38.049 | 1.00 | 97.86 | |
| ATOM | 1157 | C1′ | URI | 54 | 44.683 | 74.286 | 38.512 | 1.00 | 96.38 | |
| ATOM | 1158 | N1 | URI | 54 | 44.171 | 72.920 | 38.475 | 1.00 | 95.06 | |
| ATOM | 1159 | C6 | URI | 54 | 43.054 | 72.589 | 37.768 | 1.00 | 94.41 | |
| ATOM | 1160 | C2 | URI | 54 | 44.872 | 71.983 | 39.195 | 1.00 | 93.98 | |
| ATOM | 1161 | O2 | URI | 54 | 45.876 | 72.260 | 39.818 | 1.00 | 94.41 | |
| ATOM | 1162 | N3 | URI | 54 | 44.359 | 70.721 | 39.158 | 1.00 | 92.88 | |
| ATOM | 1163 | C4 | URI | 54 | 43.241 | 70.316 | 38.481 | 1.00 | 93.41 | |
| ATOM | 1164 | O4 | URI | 54 | 42.885 | 69.149 | 38.559 | 1.00 | 92.87 | |
| ATOM | 1165 | C5 | URI | 54 | 42.573 | 71.350 | 37.746 | 1.00 | 94.51 | |
| ATOM | 1166 | C2′ | URI | 54 | 45.913 | 74.453 | 37.631 | 1.00 | 96.75 | |
| ATOM | 1167 | O2′ | URI | 54 | 46.863 | 75.266 | 38.270 | 1.00 | 98.00 | |
| ATOM | 1168 | C3′ | URI | 54 | 45.312 | 75.078 | 36.386 | 1.00 | 97.07 | |
| ATOM | 1169 | O3′ | URI | 54 | 46.310 | 75.879 | 35.782 | 1.00 | 96.20 | |
| ATOM | 1170 | P | GUA | 55 | 47.320 | 75.217 | 34.721 | 1.00 | 96.42 | |
| ATOM | 1171 | O1P | GUA | 55 | 46.479 | 74.245 | 33.957 | 1.00 | 95.73 | |
| ATOM | 1172 | O2P | GUA | 55 | 48.033 | 76.316 | 34.001 | 1.00 | 95.06 | |
| ATOM | 1173 | O5′ | GUA | 55 | 48.387 | 74.405 | 35.596 | 1.00 | 91.98 | |
| ATOM | 1174 | C5′ | GUA | 55 | 49.552 | 75.058 | 36.077 | 1.00 | 87.15 | |
| ATOM | 1175 | C4′ | GUA | 55 | 50.255 | 74.223 | 37.117 | 1.00 | 83.54 | |
| ATOM | 1176 | O4′ | GUA | 55 | 49.265 | 73.602 | 37.979 | 1.00 | 82.94 | |
| ATOM | 1177 | C1′ | GUA | 55 | 49.732 | 72.331 | 38.408 | 1.00 | 81.80 | |
| ATOM | 1178 | N9 | GUA | 55 | 48.795 | 71.298 | 37.988 | 1.00 | 80.76 | |
| ATOM | 1179 | C4 | GUA | 55 | 48.825 | 69.974 | 38.368 | 1.00 | 78.98 | |
| ATOM | 1180 | N3 | GUA | 55 | 49.710 | 69.412 | 39.212 | 1.00 | 78.76 | |
| ATOM | 1181 | C2 | GUA | 55 | 49.487 | 68.114 | 39.376 | 1.00 | 78.70 | |
| ATOM | 1182 | N2 | GUA | 55 | 50.273 | 67.397 | 40.186 | 1.00 | 78.16 | |
| ATOM | 1183 | N1 | GUA | 55 | 48.479 | 67.422 | 38.760 | 1.00 | 77.70 | |
| ATOM | 1184 | C6 | GUA | 55 | 47.558 | 67.977 | 37.885 | 1.00 | 78.14 | |
| ATOM | 1185 | O6 | GUA | 55 | 46.689 | 67.258 | 37.378 | 1.00 | 76.59 | |
| ATOM | 1186 | C5 | GUA | 55 | 47.784 | 69.379 | 37.703 | 1.00 | 78.66 | |
| ATOM | 1187 | N7 | GUA | 55 | 47.103 | 70.310 | 36.929 | 1.00 | 80.83 | |
| ATOM | 1188 | C8 | GUA | 55 | 47.737 | 71.433 | 37.133 | 1.00 | 80.61 | |
| ATOM | 1189 | C2′ | GUA | 55 | 51.086 | 72.106 | 37.760 | 1.00 | 81.72 | |
| ATOM | 1190 | O2′ | GUA | 55 | 52.109 | 72.472 | 38.658 | 1.00 | 83.00 | |
| ATOM | 1191 | C3′ | GUA | 55 | 50.993 | 73.029 | 36.565 | 1.00 | 81.89 | |
| ATOM | 1192 | O3′ | GUA | 55 | 52.279 | 73.358 | 36.118 | 1.00 | 80.00 | |
| ATOM | 1193 | P | GUA | 56 | 52.965 | 72.410 | 35.039 | 1.00 | 79.13 | |
| ATOM | 1194 | O1P | GUA | 56 | 51.888 | 72.211 | 34.034 | 1.00 | 80.68 | |
| ATOM | 1195 | O2P | GUA | 56 | 54.296 | 72.906 | 34.610 | 1.00 | 78.75 | |
| ATOM | 1196 | O5′ | GUA | 56 | 53.176 | 71.045 | 35.823 | 1.00 | 75.87 | |
| ATOM | 1197 | C5′ | GUA | 56 | 53.997 | 71.011 | 36.969 | 1.00 | 70.12 | |
| ATOM | 1198 | C4′ | GUA | 56 | 54.344 | 69.593 | 37.318 | 1.00 | 65.83 | |
| ATOM | 1199 | O4′ | GUA | 56 | 53.193 | 68.922 | 37.907 | 1.00 | 63.10 | |
| ATOM | 1200 | C1′ | GUA | 56 | 53.197 | 67.558 | 37.536 | 1.00 | 60.13 | |
| ATOM | 1201 | N9 | GUA | 56 | 51.936 | 67.251 | 36.871 | 1.00 | 55.06 | |
| ATOM | 1202 | C4 | GUA | 56 | 51.327 | 66.022 | 36.782 | 1.00 | 51.83 | |
| ATOM | 1203 | N3 | GUA | 56 | 51.754 | 64.884 | 37.348 | 1.00 | 51.62 | |
| ATOM | 1204 | C2 | GUA | 56 | 50.954 | 63.865 | 37.063 | 1.00 | 50.36 | |
| ATOM | 1205 | N2 | GUA | 56 | 51.209 | 62.647 | 37.558 | 1.00 | 48.39 | |
| ATOM | 1206 | N1 | GUA | 56 | 49.846 | 63.971 | 36.281 | 1.00 | 49.12 | |
| ATOM | 1207 | C6 | GUA | 56 | 49.404 | 65.146 | 35.692 | 1.00 | 50.48 | |
| ATOM | 1208 | O6 | GUA | 56 | 48.397 | 65.147 | 34.996 | 1.00 | 52.46 | |
| ATOM | 1209 | C5 | GUA | 56 | 50.221 | 66.218 | 35.993 | 1.00 | 50.95 | |
| ATOM | 1210 | N7 | GUA | 56 | 50.110 | 67.543 | 35.614 | 1.00 | 52.75 | |
| ATOM | 1211 | C8 | GUA | 56 | 51.145 | 68.122 | 36.168 | 1.00 | 55.34 | |
| ATOM | 1212 | C2′ | GUA | 56 | 54.419 | 67.346 | 36.634 | 1.00 | 62.42 | |
| ATOM | 1213 | O2′ | GUA | 56 | 55.534 | 66.895 | 37.382 | 1.00 | 60.97 | |
| ATOM | 1214 | C3′ | GUA | 56 | 54.663 | 68.749 | 36.111 | 1.00 | 63.87 | |
| ATOM | 1215 | O3′ | GUA | 56 | 55.999 | 68.915 | 35.678 | 1.00 | 65.82 | |
| ATOM | 1216 | P | URI | 57 | 56.395 | 68.420 | 34.207 | 1.00 | 68.32 | |
| ATOM | 1217 | O1P | URI | 57 | 56.238 | 69.578 | 33.278 | 1.00 | 68.61 | |
| ATOM | 1218 | O2P | URI | 57 | 57.668 | 67.653 | 34.247 | 1.00 | 67.96 | |
| ATOM | 1219 | O5′ | URI | 57 | 55.208 | 67.424 | 33.857 | 1.00 | 66.91 | |
| ATOM | 1220 | C5′ | URI | 57 | 55.323 | 66.459 | 32.834 | 1.00 | 62.89 | |
| ATOM | 1221 | C4′ | URI | 57 | 55.288 | 65.104 | 33.448 | 1.00 | 61.17 | |
| ATOM | 1222 | O4′ | URI | 57 | 54.170 | 65.037 | 34.368 | 1.00 | 59.64 | |
| ATOM | 1223 | C1′ | URI | 57 | 53.624 | 63.744 | 34.344 | 1.00 | 58.14 | |
| ATOM | 1224 | N1 | URI | 57 | 52.299 | 63.823 | 33.738 | 1.00 | 56.82 | |
| ATOM | 1225 | C6 | URI | 57 | 51.839 | 64.957 | 33.104 | 1.00 | 56.14 | |
| ATOM | 1226 | C2 | URI | 57 | 51.549 | 62.684 | 33.792 | 1.00 | 56.41 | |
| ATOM | 1227 | O2 | URI | 57 | 51.938 | 61.686 | 34.367 | 1.00 | 57.08 | |
| ATOM | 1228 | N3 | URI | 57 | 50.341 | 62.749 | 33.147 | 1.00 | 55.29 | |
| ATOM | 1229 | C4 | URI | 57 | 49.828 | 63.831 | 32.473 | 1.00 | 54.85 | |
| ATOM | 1230 | O4 | URI | 57 | 48.742 | 63.726 | 31.936 | 1.00 | 55.74 | |
| ATOM | 1231 | C5 | URI | 57 | 50.656 | 64.999 | 32.477 | 1.00 | 54.31 | |
| ATOM | 1232 | C2′ | URI | 57 | 54.512 | 62.905 | 33.425 | 1.00 | 60.13 | |
| ATOM | 1233 | O2′ | URI | 57 | 55.546 | 62.312 | 34.176 | 1.00 | 60.94 | |
| ATOM | 1234 | C3′ | URI | 57 | 55.056 | 63.968 | 32.486 | 1.00 | 60.39 | |
| ATOM | 1235 | O3′ | URI | 57 | 56.277 | 63.583 | 31.890 | 1.00 | 60.40 | |
| ATOM | 1236 | P | GUA | 58 | 56.396 | 63.526 | 30.290 | 1.00 | 62.47 | |
| ATOM | 1237 | O1P | GUA | 58 | 56.106 | 64.857 | 29.696 | 1.00 | 60.25 | |
| ATOM | 1238 | O2P | GUA | 58 | 57.736 | 62.892 | 30.063 | 1.00 | 61.70 | |
| ATOM | 1239 | O5′ | GUA | 58 | 55.237 | 62.518 | 29.860 | 1.00 | 58.69 | |
| ATOM | 1240 | C5′ | GUA | 58 | 55.253 | 61.214 | 30.385 | 1.00 | 57.05 | |
| ATOM | 1241 | C4′ | GUA | 58 | 53.972 | 60.492 | 30.090 | 1.00 | 57.02 | |
| ATOM | 1242 | O4′ | GUA | 58 | 52.858 | 61.138 | 30.768 | 1.00 | 57.11 | |
| ATOM | 1243 | C1′ | GUA | 58 | 51.683 | 60.983 | 29.991 | 1.00 | 55.56 | |
| ATOM | 1244 | N9 | GUA | 58 | 51.174 | 62.295 | 29.627 | 1.00 | 54.16 | |
| ATOM | 1245 | C4 | GUA | 58 | 49.945 | 62.577 | 29.067 | 1.00 | 52.78 | |
| ATOM | 1246 | N3 | GUA | 58 | 48.966 | 61.690 | 28.807 | 1.00 | 52.89 | |
| ATOM | 1247 | C2 | GUA | 58 | 47.928 | 62.259 | 28.237 | 1.00 | 51.62 | |
| ATOM | 1248 | N2 | GUA | 58 | 46.857 | 61.517 | 27.897 | 1.00 | 51.78 | |
| ATOM | 1249 | N1 | GUA | 58 | 47.862 | 63.586 | 27.954 | 1.00 | 50.67 | |
| ATOM | 1250 | C6 | GUA | 58 | 48.871 | 64.499 | 28.202 | 1.00 | 51.62 | |
| ATOM | 1251 | O6 | GUA | 58 | 48.734 | 65.672 | 27.868 | 1.00 | 53.68 | |
| ATOM | 1252 | C5 | GUA | 58 | 49.966 | 63.911 | 28.810 | 1.00 | 50.86 | |
| ATOM | 1253 | N7 | GUA | 58 | 51.155 | 64.472 | 29.223 | 1.00 | 52.18 | |
| ATOM | 1254 | C8 | GUA | 58 | 51.837 | 63.479 | 29.713 | 1.00 | 53.06 | |
| ATOM | 1255 | C2′ | GUA | 58 | 52.082 | 60.194 | 28.744 | 1.00 | 55.93 | |
| ATOM | 1256 | O2′ | GUA | 58 | 51.952 | 58.829 | 29.058 | 1.00 | 58.80 | |
| ATOM | 1257 | C3′ | GUA | 58 | 53.558 | 60.509 | 28.634 | 1.00 | 55.57 | |
| ATOM | 1258 | O3′ | GUA | 58 | 54.234 | 59.470 | 27.970 | 1.00 | 55.26 | |
| ATOM | 1259 | P | CYT | 59 | 54.751 | 59.681 | 26.472 | 1.00 | 53.94 | |
| ATOM | 1260 | O1P | CYT | 59 | 55.211 | 61.046 | 26.193 | 1.00 | 54.90 | |
| ATOM | 1261 | O2P | CYT | 59 | 55.666 | 58.558 | 26.252 | 1.00 | 56.83 | |
| ATOM | 1262 | O5′ | CYT | 59 | 53.438 | 59.464 | 25.606 | 1.00 | 54.63 | |
| ATOM | 1263 | C5′ | CYT | 59 | 52.726 | 58.245 | 25.673 | 1.00 | 52.58 | |
| ATOM | 1264 | C4′ | CYT | 59 | 51.334 | 58.456 | 25.195 | 1.00 | 52.85 | |
| ATOM | 1265 | O4′ | CYT | 59 | 50.652 | 59.319 | 26.131 | 1.00 | 53.46 | |
| ATOM | 1266 | C1′ | CYT | 59 | 49.757 | 60.160 | 25.424 | 1.00 | 54.66 | |
| ATOM | 1267 | N1 | CYT | 59 | 50.184 | 61.554 | 25.547 | 1.00 | 55.31 | |
| ATOM | 1268 | C6 | CYT | 59 | 51.438 | 61.893 | 25.959 | 1.00 | 56.35 | |
| ATOM | 1269 | C2 | CYT | 59 | 49.285 | 62.524 | 25.200 | 1.00 | 55.52 | |
| ATOM | 1270 | O2 | CYT | 59 | 48.162 | 62.183 | 24.871 | 1.00 | 57.83 | |
| ATOM | 1271 | N3 | CYT | 59 | 49.643 | 63.806 | 25.230 | 1.00 | 56.12 | |
| ATOM | 1272 | C4 | CYT | 59 | 50.859 | 64.142 | 25.629 | 1.00 | 56.26 | |
| ATOM | 1273 | N4 | CYT | 59 | 51.151 | 65.433 | 25.671 | 1.00 | 57.73 | |
| ATOM | 1274 | C5 | CYT | 59 | 51.816 | 63.173 | 26.011 | 1.00 | 56.41 | |
| ATOM | 1275 | C2′ | CYT | 59 | 49.814 | 59.744 | 23.958 | 1.00 | 54.23 | |
| ATOM | 1276 | O2′ | CYT | 59 | 48.881 | 58.695 | 23.786 | 1.00 | 56.89 | |
| ATOM | 1277 | C3′ | CYT | 59 | 51.219 | 59.185 | 23.866 | 1.00 | 53.82 | |
| ATOM | 1278 | O3′ | CYT | 59 | 51.266 | 58.234 | 22.823 | 1.00 | 56.56 | |
| ATOM | 1279 | P | CYT | 60 | 51.920 | 58.607 | 21.413 | 1.00 | 57.74 | |
| ATOM | 1280 | O1P | CYT | 60 | 53.056 | 59.561 | 21.546 | 1.00 | 58.54 | |
| ATOM | 1281 | O2P | CYT | 60 | 52.152 | 57.283 | 20.804 | 1.00 | 59.23 | |
| ATOM | 1282 | O5′ | CYT | 60 | 50.758 | 59.360 | 20.636 | 1.00 | 57.28 | |
| ATOM | 1283 | C5′ | CYT | 60 | 49.558 | 58.697 | 20.338 | 1.00 | 58.91 | |
| ATOM | 1284 | C4′ | CYT | 60 | 48.560 | 59.664 | 19.763 | 1.00 | 61.15 | |
| ATOM | 1285 | O4′ | CYT | 60 | 48.195 | 60.652 | 20.764 | 1.00 | 62.34 | |
| ATOM | 1286 | C1′ | CYT | 60 | 47.967 | 61.893 | 20.127 | 1.00 | 63.87 | |
| ATOM | 1287 | N1 | CYT | 60 | 48.905 | 62.888 | 20.642 | 1.00 | 65.15 | |
| ATOM | 1288 | C6 | CYT | 60 | 50.055 | 62.533 | 21.279 | 1.00 | 65.50 | |
| ATOM | 1289 | C2 | CYT | 60 | 48.589 | 64.206 | 20.464 | 1.00 | 66.62 | |
| ATOM | 1290 | O2 | CYT | 60 | 47.538 | 64.482 | 19.868 | 1.00 | 69.91 | |
| ATOM | 1291 | N3 | CYT | 60 | 49.416 | 65.156 | 20.932 | 1.00 | 67.60 | |
| ATOM | 1292 | C4 | CYT | 60 | 50.535 | 64.807 | 21.560 | 1.00 | 66.68 | |
| ATOM | 1293 | N4 | CYT | 60 | 51.327 | 65.782 | 22.015 | 1.00 | 67.08 | |
| ATOM | 1294 | C5 | CYT | 60 | 50.890 | 63.454 | 21.750 | 1.00 | 65.37 | |
| ATOM | 1295 | C2′ | CYT | 60 | 48.151 | 61.687 | 18.629 | 1.00 | 63.40 | |
| ATOM | 1296 | O2′ | CYT | 60 | 46.888 | 61.366 | 18.100 | 1.00 | 66.97 | |
| ATOM | 1297 | C3′ | CYT | 60 | 49.078 | 60.485 | 18.597 | 1.00 | 62.26 | |
| ATOM | 1298 | O3′ | CYT | 60 | 48.874 | 59.753 | 17.401 | 1.00 | 63.32 | |
| ATOM | 1299 | P | ADE | 61 | 49.983 | 59.777 | 16.237 | 1.00 | 61.79 | |
| ATOM | 1300 | O1P | ADE | 61 | 51.206 | 59.119 | 16.767 | 1.00 | 62.41 | |
| ATOM | 1301 | O2P | ADE | 61 | 50.073 | 61.159 | 15.715 | 1.00 | 63.18 | |
| ATOM | 1302 | O5′ | ADE | 61 | 49.329 | 58.821 | 15.144 | 1.00 | 59.89 | |
| ATOM | 1303 | C5′ | ADE | 61 | 48.968 | 57.499 | 15.509 | 1.00 | 58.67 | |
| ATOM | 1304 | C4′ | ADE | 61 | 48.273 | 56.771 | 14.382 | 1.00 | 57.11 | |
| ATOM | 1305 | O4′ | ADE | 61 | 49.122 | 56.720 | 13.202 | 1.00 | 57.84 | |
| ATOM | 1306 | C1′ | ADE | 61 | 48.930 | 55.488 | 12.531 | 1.00 | 56.69 | |
| ATOM | 1307 | N9 | ADE | 61 | 50.225 | 54.858 | 12.327 | 1.00 | 56.48 | |
| ATOM | 1308 | C4 | ADE | 61 | 50.471 | 53.836 | 11.448 | 1.00 | 56.23 | |
| ATOM | 1309 | N3 | ADE | 61 | 49.588 | 53.226 | 10.650 | 1.00 | 55.56 | |
| ATOM | 1310 | C2 | ADE | 61 | 50.187 | 52.306 | 9.918 | 1.00 | 55.22 | |
| ATOM | 1311 | N1 | ADE | 61 | 51.467 | 51.954 | 9.891 | 1.00 | 55.55 | |
| ATOM | 1312 | C6 | ADE | 61 | 52.328 | 52.589 | 10.697 | 1.00 | 55.52 | |
| ATOM | 1313 | N6 | ADE | 61 | 53.606 | 52.241 | 10.654 | 1.00 | 55.48 | |
| ATOM | 1314 | C5 | ADE | 61 | 51.822 | 53.582 | 11.531 | 1.00 | 56.34 | |
| ATOM | 1315 | N7 | ADE | 61 | 52.422 | 54.413 | 12.465 | 1.00 | 57.42 | |
| ATOM | 1316 | C8 | ADE | 61 | 51.427 | 55.147 | 12.914 | 1.00 | 57.29 | |
| ATOM | 1317 | C2′ | ADE | 61 | 47.986 | 54.640 | 13.371 | 1.00 | 56.99 | |
| ATOM | 1318 | O2′ | ADE | 61 | 46.686 | 54.700 | 12.844 | 1.00 | 59.54 | |
| ATOM | 1319 | C3′ | ADE | 61 | 48.104 | 55.309 | 14.729 | 1.00 | 56.42 | |
| ATOM | 1320 | O3′ | ADE | 61 | 46.960 | 55.089 | 15.529 | 1.00 | 54.24 | |
| ATOM | 1321 | P | ADE | 62 | 46.930 | 53.810 | 16.468 | 1.00 | 52.98 | |
| ATOM | 1322 | O1P | ADE | 62 | 48.242 | 53.789 | 17.166 | 1.00 | 54.04 | |
| ATOM | 1323 | O2P | ADE | 62 | 45.677 | 53.754 | 17.236 | 1.00 | 55.70 | |
| ATOM | 1324 | O5′ | ADE | 62 | 46.887 | 52.609 | 15.447 | 1.00 | 53.41 | |
| ATOM | 1325 | C5′ | ADE | 62 | 45.728 | 52.367 | 14.685 | 1.00 | 53.18 | |
| ATOM | 1326 | C4′ | ADE | 62 | 45.944 | 51.154 | 13.852 | 1.00 | 54.12 | |
| ATOM | 1327 | O4′ | ADE | 62 | 47.006 | 51.438 | 12.906 | 1.00 | 53.87 | |
| ATOM | 1328 | C1′ | ADE | 62 | 47.788 | 50.280 | 12.721 | 1.00 | 53.97 | |
| ATOM | 1329 | N9 | ADE | 62 | 49.142 | 50.616 | 13.151 | 1.00 | 53.88 | |
| ATOM | 1330 | C4 | ADE | 62 | 50.332 | 50.184 | 12.607 | 1.00 | 53.44 | |
| ATOM | 1331 | N3 | ADE | 62 | 50.506 | 49.360 | 11.567 | 1.00 | 51.04 | |
| ATOM | 1332 | C2 | ADE | 62 | 51.783 | 49.129 | 11.372 | 1.00 | 51.72 | |
| ATOM | 1333 | N1 | ADE | 62 | 52.833 | 49.589 | 12.033 | 1.00 | 51.73 | |
| ATOM | 1334 | C6 | ADE | 62 | 52.629 | 50.428 | 13.062 | 1.00 | 52.48 | |
| ATOM | 1335 | N6 | ADE | 62 | 53.682 | 50.913 | 13.718 | 1.00 | 51.56 | |
| ATOM | 1336 | C5 | ADE | 62 | 51.322 | 50.748 | 13.380 | 1.00 | 53.29 | |
| ATOM | 1337 | N7 | ADE | 62 | 50.777 | 51.537 | 14.378 | 1.00 | 54.02 | |
| ATOM | 1338 | C8 | ADE | 62 | 49.483 | 51.425 | 14.196 | 1.00 | 53.66 | |
| ATOM | 1339 | C2′ | ADE | 62 | 47.142 | 49.160 | 13.554 | 1.00 | 54.57 | |
| ATOM | 1340 | O2′ | ADE | 62 | 46.169 | 48.467 | 12.807 | 1.00 | 56.26 | |
| ATOM | 1341 | C3′ | ADE | 62 | 46.425 | 49.946 | 14.634 | 1.00 | 53.80 | |
| ATOM | 1342 | O3′ | ADE | 62 | 45.288 | 49.231 | 15.067 | 1.00 | 52.14 | |
| ATOM | 1343 | P | URI | 63 | 45.452 | 47.764 | 15.708 | 1.00 | 55.62 | |
| ATOM | 1344 | O1P | URI | 63 | 44.126 | 47.083 | 15.553 | 1.00 | 52.41 | |
| ATOM | 1345 | O2P | URI | 63 | 46.687 | 47.094 | 15.199 | 1.00 | 53.35 | |
| ATOM | 1346 | O5′ | URI | 63 | 45.637 | 48.055 | 17.268 | 1.00 | 52.50 | |
| ATOM | 1347 | C5′ | URI | 63 | 44.645 | 48.789 | 17.963 | 1.00 | 53.50 | |
| ATOM | 1348 | C4′ | URI | 63 | 44.657 | 48.458 | 19.435 | 1.00 | 53.87 | |
| ATOM | 1349 | O4′ | URI | 63 | 44.222 | 47.097 | 19.638 | 1.00 | 56.21 | |
| ATOM | 1350 | C1′ | URI | 63 | 45.292 | 46.287 | 20.076 | 1.00 | 57.15 | |
| ATOM | 1351 | N1 | URI | 63 | 45.310 | 45.051 | 19.300 | 1.00 | 60.83 | |
| ATOM | 1352 | C6 | URI | 63 | 45.528 | 45.046 | 17.945 | 1.00 | 62.54 | |
| ATOM | 1353 | C2 | URI | 63 | 45.115 | 43.889 | 19.999 | 1.00 | 62.38 | |
| ATOM | 1354 | O2 | URI | 63 | 44.870 | 43.887 | 21.176 | 1.00 | 63.48 | |
| ATOM | 1355 | N3 | URI | 63 | 45.195 | 42.733 | 19.261 | 1.00 | 65.26 | |
| ATOM | 1356 | C4 | URI | 63 | 45.426 | 42.638 | 17.900 | 1.00 | 65.70 | |
| ATOM | 1357 | O4 | URI | 63 | 45.482 | 41.525 | 17.366 | 1.00 | 65.29 | |
| ATOM | 1358 | C5 | URI | 63 | 45.591 | 43.909 | 17.235 | 1.00 | 65.22 | |
| ATOM | 1359 | C2′ | URI | 63 | 46.585 | 47.075 | 20.007 | 1.00 | 54.78 | |
| ATOM | 1360 | O2′ | URI | 63 | 47.362 | 46.660 | 21.083 | 1.00 | 55.60 | |
| ATOM | 1361 | C3′ | URI | 63 | 46.057 | 48.503 | 19.996 | 1.00 | 54.61 | |
| ATOM | 1362 | O3′ | URI | 63 | 46.524 | 49.676 | 20.668 | 1.00 | 52.14 | |
| ATOM | 1363 | P | URI | 64 | 46.495 | 49.758 | 22.241 | 1.00 | 47.16 | |
| ATOM | 1364 | O1P | URI | 64 | 45.488 | 48.829 | 22.728 | 1.00 | 50.61 | |
| ATOM | 1365 | O2P | URI | 64 | 46.259 | 51.175 | 22.377 | 1.00 | 52.89 | |
| ATOM | 1366 | O5′ | URI | 64 | 47.974 | 49.293 | 22.704 | 1.00 | 48.21 | |
| ATOM | 1367 | C5′ | URI | 64 | 49.127 | 50.043 | 22.295 | 1.00 | 49.00 | |
| ATOM | 1368 | C4′ | URI | 64 | 50.424 | 49.215 | 22.264 | 1.00 | 49.69 | |
| ATOM | 1369 | O4′ | URI | 64 | 50.752 | 48.705 | 23.582 | 1.00 | 51.32 | |
| ATOM | 1370 | C1′ | URI | 64 | 51.502 | 47.527 | 23.425 | 1.00 | 48.92 | |
| ATOM | 1371 | N1 | URI | 64 | 51.387 | 46.641 | 24.575 | 1.00 | 46.53 | |
| ATOM | 1372 | C6 | URI | 64 | 50.251 | 46.555 | 25.329 | 1.00 | 47.51 | |
| ATOM | 1373 | C2 | URI | 64 | 52.513 | 45.917 | 24.883 | 1.00 | 45.36 | |
| ATOM | 1374 | O2 | URI | 64 | 53.518 | 45.952 | 24.191 | 1.00 | 41.89 | |
| ATOM | 1375 | N3 | URI | 64 | 52.416 | 45.161 | 26.021 | 1.00 | 44.24 | |
| ATOM | 1376 | C4 | URI | 64 | 51.309 | 45.060 | 26.850 | 1.00 | 46.49 | |
| ATOM | 1377 | O4 | URI | 64 | 51.397 | 44.439 | 27.899 | 1.00 | 49.41 | |
| ATOM | 1378 | C5 | URI | 64 | 50.176 | 45.806 | 26.434 | 1.00 | 45.90 | |
| ATOM | 1379 | C2′ | URI | 64 | 51.378 | 47.023 | 22.005 | 1.00 | 50.53 | |
| ATOM | 1380 | O2′ | URI | 64 | 52.586 | 47.198 | 21.311 | 1.00 | 55.33 | |
| ATOM | 1381 | C3′ | URI | 64 | 50.320 | 47.949 | 21.432 | 1.00 | 51.44 | |
| ATOM | 1382 | O3′ | URI | 64 | 50.896 | 48.097 | 20.133 | 1.00 | 54.21 | |
| ATOM | 1383 | P | CYT | 65 | 51.317 | 49.531 | 19.527 | 1.00 | 53.17 | |
| ATOM | 1384 | O1P | CYT | 65 | 50.384 | 50.624 | 19.859 | 1.00 | 55.94 | |
| ATOM | 1385 | O2P | CYT | 65 | 52.765 | 49.732 | 19.678 | 1.00 | 52.59 | |
| ATOM | 1386 | O5′ | CYT | 65 | 50.960 | 49.263 | 18.006 | 1.00 | 53.37 | |
| ATOM | 1387 | C5′ | CYT | 65 | 49.601 | 48.993 | 17.681 | 1.00 | 53.06 | |
| ATOM | 1388 | C4′ | CYT | 65 | 49.485 | 47.895 | 16.662 | 1.00 | 52.33 | |
| ATOM | 1389 | O4′ | CYT | 65 | 50.188 | 48.278 | 15.454 | 1.00 | 53.26 | |
| ATOM | 1390 | C1′ | CYT | 65 | 50.746 | 47.130 | 14.856 | 1.00 | 51.98 | |
| ATOM | 1391 | N1 | CYT | 65 | 52.185 | 47.288 | 14.767 | 1.00 | 50.86 | |
| ATOM | 1392 | C6 | CYT | 65 | 52.860 | 48.201 | 15.517 | 1.00 | 50.95 | |
| ATOM | 1393 | C2 | CYT | 65 | 52.841 | 46.474 | 13.912 | 1.00 | 50.54 | |
| ATOM | 1394 | O2 | CYT | 65 | 52.180 | 45.668 | 13.289 | 1.00 | 51.03 | |
| ATOM | 1395 | N3 | CYT | 65 | 54.176 | 46.569 | 13.778 | 1.00 | 51.62 | |
| ATOM | 1396 | C4 | CYT | 65 | 54.846 | 47.461 | 14.496 | 1.00 | 51.59 | |
| ATOM | 1397 | N4 | CYT | 65 | 56.159 | 47.552 | 14.311 | 1.00 | 49.49 | |
| ATOM | 1398 | C5 | CYT | 65 | 54.190 | 48.312 | 15.424 | 1.00 | 51.97 | |
| ATOM | 1399 | C2′ | CYT | 65 | 50.366 | 45.930 | 15.713 | 1.00 | 51.84 | |
| ATOM | 1400 | O2′ | CYT | 65 | 49.194 | 45.396 | 15.175 | 1.00 | 53.41 | |
| ATOM | 1401 | C3′ | CYT | 65 | 50.108 | 46.582 | 17.060 | 1.00 | 50.91 | |
| ATOM | 1402 | O3′ | CYT | 65 | 49.129 | 45.857 | 17.768 | 1.00 | 50.92 | |
| ATOM | 1403 | P | CYT | 66 | 49.562 | 44.593 | 18.655 | 1.00 | 48.98 | |
| ATOM | 1404 | O1P | CYT | 66 | 50.826 | 44.962 | 19.331 | 1.00 | 51.50 | |
| ATOM | 1405 | O2P | CYT | 66 | 48.390 | 44.151 | 19.452 | 1.00 | 48.66 | |
| ATOM | 1406 | O5′ | CYT | 66 | 49.913 | 43.489 | 17.582 | 1.00 | 49.32 | |
| ATOM | 1407 | C5′ | CYT | 66 | 48.881 | 42.819 | 16.869 | 1.00 | 49.81 | |
| ATOM | 1408 | C4′ | CYT | 66 | 49.493 | 41.817 | 15.923 | 1.00 | 49.64 | |
| ATOM | 1409 | O4′ | CYT | 66 | 50.398 | 42.503 | 15.040 | 1.00 | 49.04 | |
| ATOM | 1410 | C1′ | CYT | 66 | 51.491 | 41.672 | 14.748 | 1.00 | 49.15 | |
| ATOM | 1411 | N1 | CYT | 66 | 52.690 | 42.355 | 15.147 | 1.00 | 49.29 | |
| ATOM | 1412 | C6 | CYT | 66 | 52.670 | 43.422 | 15.996 | 1.00 | 49.77 | |
| ATOM | 1413 | C2 | CYT | 66 | 53.847 | 41.903 | 14.628 | 1.00 | 49.70 | |
| ATOM | 1414 | O2 | CYT | 66 | 53.783 | 40.941 | 13.875 | 1.00 | 52.12 | |
| ATOM | 1415 | N3 | CYT | 66 | 55.005 | 42.513 | 14.932 | 1.00 | 49.35 | |
| ATOM | 1416 | C4 | CYT | 66 | 55.000 | 43.573 | 15.723 | 1.00 | 49.49 | |
| ATOM | 1417 | N4 | CYT | 66 | 56.161 | 44.176 | 15.956 | 1.00 | 50.87 | |
| ATOM | 1418 | C5 | CYT | 66 | 53.800 | 44.068 | 16.299 | 1.00 | 50.40 | |
| ATOM | 1419 | C2′ | CYT | 66 | 51.335 | 40.391 | 15.534 | 1.00 | 50.40 | |
| ATOM | 1420 | O2′ | CYT | 66 | 50.768 | 39.402 | 14.712 | 1.00 | 55.45 | |
| ATOM | 1421 | C3′ | CYT | 66 | 50.401 | 40.845 | 16.619 | 1.00 | 50.61 | |
| ATOM | 1422 | O3′ | CYT | 66 | 49.654 | 39.771 | 17.066 | 1.00 | 53.83 | |
| ATOM | 1423 | P | URI | 67 | 50.198 | 38.958 | 18.309 | 1.00 | 60.29 | |
| ATOM | 1424 | O1P | URI | 67 | 50.458 | 40.026 | 19.327 | 1.00 | 60.61 | |
| ATOM | 1425 | O2P | URI | 67 | 49.233 | 37.845 | 18.604 | 1.00 | 58.42 | |
| ATOM | 1426 | O5′ | URI | 67 | 51.635 | 38.440 | 17.831 | 1.00 | 57.51 | |
| ATOM | 1427 | C5′ | URI | 67 | 51.757 | 37.391 | 16.908 | 1.00 | 58.65 | |
| ATOM | 1428 | C4′ | URI | 67 | 53.201 | 37.032 | 16.721 | 1.00 | 61.28 | |
| ATOM | 1429 | O4′ | URI | 67 | 53.940 | 38.184 | 16.235 | 1.00 | 61.89 | |
| ATOM | 1430 | C1′ | URI | 67 | 55.248 | 38.167 | 16.769 | 1.00 | 60.63 | |
| ATOM | 1431 | N1 | URI | 67 | 55.452 | 39.449 | 17.455 | 1.00 | 60.26 | |
| ATOM | 1432 | C6 | URI | 67 | 54.438 | 40.042 | 18.169 | 1.00 | 59.35 | |
| ATOM | 1433 | C2 | URI | 67 | 56.672 | 40.067 | 17.326 | 1.00 | 60.95 | |
| ATOM | 1434 | O2 | URI | 67 | 57.623 | 39.546 | 16.788 | 1.00 | 63.34 | |
| ATOM | 1435 | N3 | URI | 67 | 56.741 | 41.323 | 17.865 | 1.00 | 59.53 | |
| ATOM | 1436 | C4 | URI | 67 | 55.746 | 41.990 | 18.533 | 1.00 | 58.08 | |
| ATOM | 1437 | O4 | URI | 67 | 55.907 | 43.170 | 18.808 | 1.00 | 59.78 | |
| ATOM | 1438 | C5 | URI | 67 | 54.547 | 41.258 | 18.703 | 1.00 | 57.16 | |
| ATOM | 1439 | C2′ | URI | 67 | 55.367 | 36.909 | 17.637 | 1.00 | 61.57 | |
| ATOM | 1440 | O2′ | URI | 67 | 55.845 | 35.839 | 16.881 | 1.00 | 62.01 | |
| ATOM | 1441 | C3′ | URI | 67 | 53.922 | 36.616 | 17.984 | 1.00 | 62.55 | |
| ATOM | 1442 | O3′ | URI | 67 | 53.727 | 35.213 | 18.130 | 1.00 | 67.86 | |
| ATOM | 1443 | P | GUA | 68 | 54.264 | 34.436 | 19.440 | 1.00 | 72.38 | |
| ATOM | 1444 | O1P | GUA | 68 | 53.770 | 35.152 | 20.655 | 1.00 | 71.00 | |
| ATOM | 1445 | O2P | GUA | 68 | 53.893 | 33.003 | 19.254 | 1.00 | 71.11 | |
| ATOM | 1446 | O5′ | GUA | 68 | 55.852 | 34.618 | 19.347 | 1.00 | 72.46 | |
| ATOM | 1447 | C5′ | GUA | 68 | 56.705 | 33.576 | 18.884 | 1.00 | 76.11 | |
| ATOM | 1448 | C4′ | GUA | 68 | 58.114 | 33.823 | 19.375 | 1.00 | 80.18 | |
| ATOM | 1449 | O4′ | GUA | 68 | 58.509 | 35.142 | 18.923 | 1.00 | 80.39 | |
| ATOM | 1450 | C1′ | GUA | 68 | 59.293 | 35.770 | 19.921 | 1.00 | 82.59 | |
| ATOM | 1451 | N9 | GUA | 68 | 58.629 | 37.008 | 20.322 | 1.00 | 82.78 | |
| ATOM | 1452 | C4 | GUA | 68 | 59.218 | 38.247 | 20.382 | 1.00 | 82.04 | |
| ATOM | 1453 | N3 | GUA | 68 | 60.510 | 38.512 | 20.128 | 1.00 | 82.48 | |
| ATOM | 1454 | C2 | GUA | 68 | 60.783 | 39.796 | 20.230 | 1.00 | 82.61 | |
| ATOM | 1455 | N2 | GUA | 68 | 62.043 | 40.223 | 20.024 | 1.00 | 82.03 | |
| ATOM | 1456 | N1 | GUA | 68 | 59.852 | 40.750 | 20.542 | 1.00 | 82.74 | |
| ATOM | 1457 | C6 | GUA | 68 | 58.517 | 40.501 | 20.811 | 1.00 | 82.22 | |
| ATOM | 1458 | O6 | GUA | 68 | 57.772 | 41.441 | 21.085 | 1.00 | 83.18 | |
| ATOM | 1459 | C5 | GUA | 68 | 58.214 | 39.117 | 20.720 | 1.00 | 81.67 | |
| ATOM | 1460 | N7 | GUA | 68 | 57.018 | 38.436 | 20.911 | 1.00 | 82.42 | |
| ATOM | 1461 | C8 | GUA | 68 | 57.313 | 37.185 | 20.674 | 1.00 | 82.57 | |
| ATOM | 1462 | C2′ | GUA | 68 | 59.497 | 34.769 | 21.056 | 1.00 | 83.40 | |
| ATOM | 1463 | O2′ | GUA | 68 | 60.727 | 34.088 | 20.861 | 1.00 | 86.48 | |
| ATOM | 1464 | C3′ | GUA | 68 | 58.279 | 33.869 | 20.893 | 1.00 | 82.31 | |
| ATOM | 1465 | O3′ | GUA | 68 | 58.587 | 32.612 | 21.544 | 1.00 | 83.68 | |
| ATOM | 1466 | P | CYT | 69 | 59.369 | 31.399 | 20.769 | 1.00 | 84.23 | |
| ATOM | 1467 | O1P | CYT | 69 | 58.420 | 30.647 | 19.889 | 1.00 | 83.58 | |
| ATOM | 1468 | O2P | CYT | 69 | 60.716 | 31.808 | 20.230 | 1.00 | 80.34 | |
| ATOM | 1469 | O5′ | CYT | 69 | 59.625 | 30.383 | 21.968 | 1.00 | 82.21 | |
| ATOM | 1470 | C5′ | CYT | 69 | 60.461 | 30.737 | 23.061 | 1.00 | 78.76 | |
| ATOM | 1471 | C4′ | CYT | 69 | 59.732 | 30.511 | 24.355 | 1.00 | 76.22 | |
| ATOM | 1472 | O4′ | CYT | 69 | 59.054 | 31.736 | 24.754 | 1.00 | 74.55 | |
| ATOM | 1473 | C1′ | CYT | 69 | 57.846 | 31.403 | 25.421 | 1.00 | 73.25 | |
| ATOM | 1474 | N1 | CYT | 69 | 56.716 | 32.049 | 24.752 | 1.00 | 71.20 | |
| ATOM | 1475 | C6 | CYT | 69 | 56.858 | 32.670 | 23.547 | 1.00 | 69.73 | |
| ATOM | 1476 | C2 | CYT | 69 | 55.475 | 32.026 | 25.395 | 1.00 | 70.95 | |
| ATOM | 1477 | O2 | CYT | 69 | 55.374 | 31.413 | 26.476 | 1.00 | 71.58 | |
| ATOM | 1478 | N3 | CYT | 69 | 54.428 | 32.653 | 24.830 | 1.00 | 68.71 | |
| ATOM | 1479 | C4 | CYT | 69 | 54.585 | 33.273 | 23.664 | 1.00 | 68.96 | |
| ATOM | 1480 | N4 | CYT | 69 | 53.533 | 33.895 | 23.150 | 1.00 | 70.36 | |
| ATOM | 1481 | C5 | CYT | 69 | 55.832 | 33.287 | 22.973 | 1.00 | 68.05 | |
| ATOM | 1482 | C2′ | CYT | 69 | 57.716 | 29.882 | 25.429 | 1.00 | 74.80 | |
| ATOM | 1483 | O2′ | CYT | 69 | 58.217 | 29.348 | 26.644 | 1.00 | 74.46 | |
| ATOM | 1484 | C3′ | CYT | 69 | 58.597 | 29.504 | 24.251 | 1.00 | 75.79 | |
| ATOM | 1485 | O3′ | CYT | 69 | 59.017 | 28.156 | 24.376 | 1.00 | 76.78 | |
| ATOM | 1486 | P | ADE | 70 | 57.989 | 26.969 | 23.996 | 1.00 | 76.95 | |
| ATOM | 1487 | O1P | ADE | 70 | 57.499 | 27.272 | 22.618 | 1.00 | 77.97 | |
| ATOM | 1488 | O2P | ADE | 70 | 58.641 | 25.672 | 24.283 | 1.00 | 76.26 | |
| ATOM | 1489 | O5′ | ADE | 70 | 56.782 | 27.097 | 25.027 | 1.00 | 73.11 | |
| ATOM | 1490 | C5′ | ADE | 70 | 56.891 | 26.459 | 26.268 | 1.00 | 71.25 | |
| ATOM | 1491 | C4′ | ADE | 70 | 55.612 | 26.557 | 27.033 | 1.00 | 70.06 | |
| ATOM | 1492 | O4′ | ADE | 70 | 55.235 | 27.954 | 27.125 | 1.00 | 69.60 | |
| ATOM | 1493 | C1′ | ADE | 70 | 53.824 | 28.065 | 27.118 | 1.00 | 67.58 | |
| ATOM | 1494 | N9 | ADE | 70 | 53.424 | 28.901 | 25.988 | 1.00 | 63.61 | |
| ATOM | 1495 | C4 | ADE | 70 | 52.169 | 29.419 | 25.812 | 1.00 | 60.89 | |
| ATOM | 1496 | N3 | ADE | 70 | 51.112 | 29.286 | 26.638 | 1.00 | 60.32 | |
| ATOM | 1497 | C2 | ADE | 70 | 50.036 | 29.902 | 26.138 | 1.00 | 57.84 | |
| ATOM | 1498 | N1 | ADE | 70 | 49.913 | 30.575 | 24.998 | 1.00 | 57.55 | |
| ATOM | 1499 | C6 | ADE | 70 | 51.004 | 30.702 | 24.191 | 1.00 | 59.94 | |
| ATOM | 1500 | N6 | ADE | 70 | 50.888 | 31.396 | 23.045 | 1.00 | 59.94 | |
| ATOM | 1501 | C5 | ADE | 70 | 52.204 | 30.096 | 24.611 | 1.00 | 60.12 | |
| ATOM | 1502 | N7 | ADE | 70 | 53.468 | 30.028 | 24.045 | 1.00 | 60.62 | |
| ATOM | 1503 | C8 | ADE | 70 | 54.156 | 29.309 | 24.902 | 1.00 | 62.12 | |
| ATOM | 1504 | C2′ | ADE | 70 | 53.257 | 26.645 | 27.024 | 1.00 | 69.11 | |
| ATOM | 1505 | O2′ | ADE | 70 | 52.988 | 26.150 | 28.313 | 1.00 | 68.84 | |
| ATOM | 1506 | C3′ | ADE | 70 | 54.417 | 25.902 | 26.374 | 1.00 | 70.69 | |
| ATOM | 1507 | O3′ | ADE | 70 | 54.378 | 24.499 | 26.631 | 1.00 | 72.73 | |
| ATOM | 1508 | P | GUA | 71 | 53.276 | 23.580 | 25.896 | 1.00 | 72.87 | |
| ATOM | 1509 | O1P | GUA | 71 | 53.264 | 23.884 | 24.445 | 1.00 | 75.88 | |
| ATOM | 1510 | O2P | GUA | 71 | 53.442 | 22.190 | 26.343 | 1.00 | 76.18 | |
| ATOM | 1511 | O5′ | GUA | 71 | 51.901 | 24.057 | 26.498 | 1.00 | 71.48 | |
| ATOM | 1512 | C5′ | GUA | 71 | 51.534 | 23.643 | 27.776 | 1.00 | 71.45 | |
| ATOM | 1513 | C4′ | GUA | 71 | 50.139 | 24.073 | 28.047 | 1.00 | 73.04 | |
| ATOM | 1514 | O4′ | GUA | 71 | 50.058 | 25.500 | 27.788 | 1.00 | 73.17 | |
| ATOM | 1515 | C1′ | GUA | 71 | 48.778 | 25.812 | 27.260 | 1.00 | 73.14 | |
| ATOM | 1516 | N9 | GUA | 71 | 48.932 | 26.515 | 25.980 | 1.00 | 72.35 | |
| ATOM | 1517 | C4 | GUA | 71 | 47.928 | 27.175 | 25.303 | 1.00 | 71.25 | |
| ATOM | 1518 | N3 | GUA | 71 | 46.633 | 27.233 | 25.678 | 1.00 | 71.18 | |
| ATOM | 1519 | C2 | GUA | 71 | 45.907 | 27.970 | 24.846 | 1.00 | 71.64 | |
| ATOM | 1520 | N2 | GUA | 71 | 44.587 | 28.135 | 25.086 | 1.00 | 70.78 | |
| ATOM | 1521 | N1 | GUA | 71 | 46.418 | 28.598 | 23.728 | 1.00 | 70.00 | |
| ATOM | 1522 | C6 | GUA | 71 | 47.747 | 28.545 | 23.317 | 1.00 | 70.90 | |
| ATOM | 1523 | O6 | GUA | 71 | 48.099 | 29.146 | 22.286 | 1.00 | 70.86 | |
| ATOM | 1524 | C5 | GUA | 71 | 48.538 | 27.757 | 24.206 | 1.00 | 70.76 | |
| ATOM | 1525 | N7 | GUA | 71 | 49.893 | 27.443 | 24.172 | 1.00 | 71.38 | |
| ATOM | 1526 | C8 | GUA | 71 | 50.081 | 26.696 | 25.236 | 1.00 | 72.00 | |
| ATOM | 1527 | C2′ | GUA | 71 | 47.999 | 24.500 | 27.188 | 1.00 | 73.72 | |
| ATOM | 1528 | O2′ | GUA | 71 | 47.325 | 24.284 | 28.417 | 1.00 | 72.42 | |
| ATOM | 1529 | C3′ | GUA | 71 | 49.135 | 23.510 | 27.067 | 1.00 | 73.71 | |
| ATOM | 1530 | O3′ | GUA | 71 | 48.757 | 22.183 | 27.341 | 1.00 | 75.61 | |
| ATOM | 1531 | P | CYT | 72 | 48.600 | 21.161 | 26.116 | 1.00 | 76.77 | |
| ATOM | 1532 | O1P | CYT | 72 | 49.717 | 21.470 | 25.171 | 1.00 | 76.59 | |
| ATOM | 1533 | O2P | CYT | 72 | 48.454 | 19.787 | 26.646 | 1.00 | 78.76 | |
| ATOM | 1534 | O5′ | CYT | 72 | 47.170 | 21.529 | 25.531 | 1.00 | 75.72 | |
| ATOM | 1535 | C5′ | CYT | 72 | 46.022 | 21.217 | 26.299 | 1.00 | 74.12 | |
| ATOM | 1536 | C4′ | CYT | 72 | 44.819 | 21.972 | 25.801 | 1.00 | 74.81 | |
| ATOM | 1537 | O4′ | CYT | 72 | 45.131 | 23.392 | 25.774 | 1.00 | 73.56 | |
| ATOM | 1538 | C1′ | CYT | 72 | 44.468 | 24.018 | 24.682 | 1.00 | 69.92 | |
| ATOM | 1539 | N1 | CYT | 72 | 45.486 | 24.530 | 23.760 | 1.00 | 64.17 | |
| ATOM | 1540 | C6 | CYT | 72 | 46.773 | 24.101 | 23.845 | 1.00 | 60.21 | |
| ATOM | 1541 | C2 | CYT | 72 | 45.113 | 25.481 | 22.790 | 1.00 | 61.28 | |
| ATOM | 1542 | O2 | CYT | 72 | 43.928 | 25.861 | 22.746 | 1.00 | 59.77 | |
| ATOM | 1543 | N3 | CYT | 72 | 46.047 | 25.959 | 21.945 | 1.00 | 57.28 | |
| ATOM | 1544 | C4 | CYT | 72 | 47.303 | 25.530 | 22.044 | 1.00 | 57.49 | |
| ATOM | 1545 | N4 | CYT | 72 | 48.204 | 26.015 | 21.189 | 1.00 | 57.41 | |
| ATOM | 1546 | C5 | CYT | 72 | 47.702 | 24.575 | 23.023 | 1.00 | 58.34 | |
| ATOM | 1547 | C2′ | CYT | 72 | 43.600 | 22.952 | 24.033 | 1.00 | 73.14 | |
| ATOM | 1548 | O2′ | CYT | 72 | 42.348 | 22.991 | 24.703 | 1.00 | 73.44 | |
| ATOM | 1549 | C3′ | CYT | 72 | 44.407 | 21.707 | 24.369 | 1.00 | 75.16 | |
| ATOM | 1550 | O3′ | CYT | 72 | 43.744 | 20.475 | 24.191 | 1.00 | 78.26 | |
| ATOM | 1551 | P | GUA | 73 | 44.244 | 19.510 | 23.012 | 1.00 | 80.74 | |
| ATOM | 1552 | O1P | GUA | 73 | 45.708 | 19.778 | 22.826 | 1.00 | 79.68 | |
| ATOM | 1553 | O2P | GUA | 73 | 43.777 | 18.122 | 23.296 | 1.00 | 81.29 | |
| ATOM | 1554 | O5′ | GUA | 73 | 43.456 | 20.082 | 21.750 | 1.00 | 79.14 | |
| ATOM | 1555 | C5′ | GUA | 73 | 42.097 | 20.488 | 21.888 | 1.00 | 76.72 | |
| ATOM | 1556 | C4′ | GUA | 73 | 41.706 | 21.420 | 20.768 | 1.00 | 75.29 | |
| ATOM | 1557 | O4′ | GUA | 73 | 42.431 | 22.676 | 20.868 | 1.00 | 73.14 | |
| ATOM | 1558 | C1′ | GUA | 73 | 42.538 | 23.256 | 19.582 | 1.00 | 70.85 | |
| ATOM | 1559 | N9 | GUA | 73 | 43.941 | 23.537 | 19.290 | 1.00 | 67.16 | |
| ATOM | 1560 | C4 | GUA | 73 | 44.378 | 24.361 | 18.285 | 1.00 | 65.56 | |
| ATOM | 1561 | N3 | GUA | 73 | 43.590 | 25.041 | 17.437 | 1.00 | 65.02 | |
| ATOM | 1562 | C2 | GUA | 73 | 44.286 | 25.733 | 16.550 | 1.00 | 64.36 | |
| ATOM | 1563 | N2 | GUA | 73 | 43.631 | 26.443 | 15.633 | 1.00 | 65.45 | |
| ATOM | 1564 | N1 | GUA | 73 | 45.658 | 25.774 | 16.493 | 1.00 | 61.57 | |
| ATOM | 1565 | C6 | GUA | 73 | 46.507 | 25.096 | 17.352 | 1.00 | 62.56 | |
| ATOM | 1566 | O6 | GUA | 73 | 47.741 | 25.209 | 17.204 | 1.00 | 60.52 | |
| ATOM | 1567 | C5 | GUA | 73 | 45.757 | 24.320 | 18.336 | 1.00 | 64.44 | |
| ATOM | 1568 | N7 | GUA | 73 | 46.182 | 23.487 | 19.374 | 1.00 | 63.69 | |
| ATOM | 1569 | C8 | GUA | 73 | 45.068 | 23.051 | 19.914 | 1.00 | 65.34 | |
| ATOM | 1570 | C2′ | GUA | 73 | 41.915 | 22.260 | 18.599 | 1.00 | 73.56 | |
| ATOM | 1571 | O2′ | GUA | 73 | 40.549 | 22.580 | 18.423 | 1.00 | 73.84 | |
| ATOM | 1572 | C3′ | GUA | 73 | 42.066 | 20.959 | 19.372 | 1.00 | 74.93 | |
| ATOM | 1573 | O3′ | GUA | 73 | 41.170 | 19.956 | 18.926 | 1.00 | 75.65 | |
| ATOM | 1574 | P | GUA | 74 | 41.755 | 18.537 | 18.438 | 1.00 | 76.59 | |
| ATOM | 1575 | O1P | GUA | 74 | 43.134 | 18.335 | 18.985 | 1.00 | 74.18 | |
| ATOM | 1576 | O2P | GUA | 74 | 40.680 | 17.519 | 18.681 | 1.00 | 77.25 | |
| ATOM | 1577 | O5′ | GUA | 74 | 41.946 | 18.711 | 16.875 | 1.00 | 74.44 | |
| ATOM | 1578 | C5′ | GUA | 74 | 42.942 | 19.584 | 16.384 | 1.00 | 72.28 | |
| ATOM | 1579 | C4′ | GUA | 74 | 42.523 | 20.110 | 15.061 | 1.00 | 69.30 | |
| ATOM | 1580 | O4′ | GUA | 74 | 42.815 | 21.529 | 15.065 | 1.00 | 69.43 | |
| ATOM | 1581 | C1′ | GUA | 74 | 43.928 | 21.824 | 14.235 | 1.00 | 66.32 | |
| ATOM | 1582 | N9 | GUA | 74 | 45.069 | 22.121 | 15.094 | 1.00 | 62.89 | |
| ATOM | 1583 | C4 | GUA | 74 | 46.252 | 22.742 | 14.730 | 1.00 | 60.17 | |
| ATOM | 1584 | N3 | GUA | 74 | 46.491 | 23.412 | 13.584 | 1.00 | 58.28 | |
| ATOM | 1585 | C2 | GUA | 74 | 47.766 | 23.777 | 13.483 | 1.00 | 59.10 | |
| ATOM | 1586 | N2 | GUA | 74 | 48.213 | 24.468 | 12.420 | 1.00 | 59.53 | |
| ATOM | 1587 | N1 | GUA | 74 | 48.710 | 23.495 | 14.416 | 1.00 | 59.95 | |
| ATOM | 1588 | C6 | GUA | 74 | 48.476 | 22.811 | 15.599 | 1.00 | 61.04 | |
| ATOM | 1589 | O6 | GUA | 74 | 49.401 | 22.600 | 16.374 | 1.00 | 64.54 | |
| ATOM | 1590 | C5 | GUA | 74 | 47.134 | 22.435 | 15.733 | 1.00 | 60.25 | |
| ATOM | 1591 | N7 | GUA | 74 | 46.496 | 21.764 | 16.765 | 1.00 | 59.39 | |
| ATOM | 1592 | C8 | GUA | 74 | 45.261 | 21.646 | 16.363 | 1.00 | 60.14 | |
| ATOM | 1593 | C2′ | GUA | 74 | 44.338 | 20.550 | 13.480 | 1.00 | 67.90 | |
| ATOM | 1594 | O2′ | GUA | 74 | 44.244 | 20.810 | 12.091 | 1.00 | 67.95 | |
| ATOM | 1595 | C3′ | GUA | 74 | 43.274 | 19.548 | 13.881 | 1.00 | 68.61 | |
| ATOM | 1596 | O3′ | GUA | 74 | 42.954 | 18.335 | 13.203 | 1.00 | 71.06 | |
| ATOM | 1597 | P | ADE | 75 | 43.965 | 17.056 | 13.292 | 1.00 | 72.54 | |
| ATOM | 1598 | O1P | ADE | 75 | 44.354 | 16.785 | 14.697 | 1.00 | 73.48 | |
| ATOM | 1599 | O2P | ADE | 75 | 43.315 | 15.988 | 12.498 | 1.00 | 74.45 | |
| ATOM | 1600 | O5′ | ADE | 75 | 45.321 | 17.464 | 12.551 | 1.00 | 69.45 | |
| ATOM | 1601 | C5′ | ADE | 75 | 46.455 | 16.600 | 12.592 | 1.00 | 66.56 | |
| ATOM | 1602 | C4′ | ADE | 75 | 47.390 | 16.916 | 11.449 | 1.00 | 66.48 | |
| ATOM | 1603 | O4′ | ADE | 75 | 46.796 | 16.534 | 10.188 | 1.00 | 67.66 | |
| ATOM | 1604 | C1′ | ADE | 75 | 46.920 | 17.591 | 9.249 | 1.00 | 65.79 | |
| ATOM | 1605 | N9 | ADE | 75 | 45.583 | 18.117 | 9.033 | 1.00 | 65.93 | |
| ATOM | 1606 | C4 | ADE | 75 | 45.142 | 18.909 | 8.008 | 1.00 | 66.83 | |
| ATOM | 1607 | N3 | ADE | 75 | 45.860 | 19.389 | 6.984 | 1.00 | 69.04 | |
| ATOM | 1608 | C2 | ADE | 75 | 45.096 | 20.123 | 6.174 | 1.00 | 68.75 | |
| ATOM | 1609 | N1 | ADE | 75 | 43.793 | 20.408 | 6.272 | 1.00 | 69.10 | |
| ATOM | 1610 | C6 | ADE | 75 | 43.103 | 19.914 | 7.317 | 1.00 | 67.87 | |
| ATOM | 1611 | N6 | ADE | 75 | 41.807 | 20.224 | 7.423 | 1.00 | 68.55 | |
| ATOM | 1612 | C5 | ADE | 75 | 43.799 | 19.109 | 8.239 | 1.00 | 67.73 | |
| ATOM | 1613 | N7 | ADE | 75 | 43.399 | 18.446 | 9.392 | 1.00 | 68.62 | |
| ATOM | 1614 | C8 | ADE | 75 | 44.493 | 17.883 | 9.825 | 1.00 | 65.84 | |
| ATOM | 1615 | C2′ | ADE | 75 | 47.892 | 18.598 | 9.836 | 1.00 | 65.58 | |
| ATOM | 1616 | O2′ | ADE | 75 | 49.202 | 18.242 | 9.484 | 1.00 | 67.52 | |
| ATOM | 1617 | C3′ | ADE | 75 | 47.674 | 18.393 | 11.322 | 1.00 | 66.29 | |
| ATOM | 1618 | O3′ | ADE | 75 | 48.847 | 18.682 | 12.037 | 1.00 | 64.47 | |
| ATOM | 1619 | P | ADE | 76 | 48.942 | 20.053 | 12.823 | 1.00 | 63.80 | |
| ATOM | 1620 | O1P | ADE | 76 | 47.615 | 20.189 | 13.506 | 1.00 | 60.84 | |
| ATOM | 1621 | O2P | ADE | 76 | 50.194 | 20.075 | 13.623 | 1.00 | 64.05 | |
| ATOM | 1622 | O5′ | ADE | 76 | 49.103 | 21.110 | 11.638 | 1.00 | 62.31 | |
| ATOM | 1623 | C5′ | ADE | 76 | 50.353 | 21.268 | 10.971 | 1.00 | 59.37 | |
| ATOM | 1624 | C4′ | ADE | 76 | 50.144 | 21.990 | 9.672 | 1.00 | 58.58 | |
| ATOM | 1625 | O4′ | ADE | 76 | 48.985 | 21.401 | 9.022 | 1.00 | 58.49 | |
| ATOM | 1626 | C1′ | ADE | 76 | 48.264 | 22.397 | 8.334 | 1.00 | 55.92 | |
| ATOM | 1627 | N9 | ADE | 76 | 46.916 | 22.378 | 8.865 | 1.00 | 54.24 | |
| ATOM | 1628 | C4 | ADE | 76 | 45.781 | 22.771 | 8.203 | 1.00 | 54.53 | |
| ATOM | 1629 | N3 | ADE | 76 | 45.704 | 23.298 | 6.968 | 1.00 | 53.24 | |
| ATOM | 1630 | C2 | ADE | 76 | 44.439 | 23.539 | 6.649 | 1.00 | 53.99 | |
| ATOM | 1631 | N1 | ADE | 76 | 43.321 | 23.317 | 7.367 | 1.00 | 54.54 | |
| ATOM | 1632 | C6 | ADE | 76 | 43.439 | 22.770 | 8.599 | 1.00 | 53.56 | |
| ATOM | 1633 | N6 | ADE | 76 | 42.333 | 22.538 | 9.301 | 1.00 | 53.61 | |
| ATOM | 1634 | C5 | ADE | 76 | 44.726 | 22.482 | 9.058 | 1.00 | 53.52 | |
| ATOM | 1635 | N7 | ADE | 76 | 45.194 | 21.943 | 10.242 | 1.00 | 53.28 | |
| ATOM | 1636 | C8 | ADE | 76 | 46.500 | 21.917 | 10.084 | 1.00 | 53.25 | |
| ATOM | 1637 | C2′ | ADE | 76 | 49.020 | 23.715 | 8.499 | 1.00 | 57.18 | |
| ATOM | 1638 | O2′ | ADE | 76 | 49.845 | 23.921 | 7.361 | 1.00 | 54.08 | |
| ATOM | 1639 | C3′ | ADE | 76 | 49.787 | 23.460 | 9.797 | 1.00 | 57.81 | |
| ATOM | 1640 | O3′ | ADE | 76 | 50.988 | 24.224 | 9.855 | 1.00 | 58.36 | |
| ATOM | 1641 | P | ADE | 77 | 51.005 | 25.633 | 10.638 | 1.00 | 58.98 | |
| ATOM | 1642 | O1P | ADE | 77 | 50.449 | 25.423 | 12.011 | 1.00 | 57.84 | |
| ATOM | 1643 | O2P | ADE | 77 | 52.349 | 26.222 | 10.476 | 1.00 | 57.35 | |
| ATOM | 1644 | O5′ | ADE | 77 | 50.003 | 26.527 | 9.774 | 1.00 | 59.45 | |
| ATOM | 1645 | C5′ | ADE | 77 | 50.461 | 27.157 | 8.572 | 1.00 | 57.78 | |
| ATOM | 1646 | C4′ | ADE | 77 | 49.294 | 27.665 | 7.746 | 1.00 | 57.17 | |
| ATOM | 1647 | O4′ | ADE | 77 | 48.353 | 26.582 | 7.507 | 1.00 | 57.75 | |
| ATOM | 1648 | C1′ | ADE | 77 | 47.031 | 27.084 | 7.520 | 1.00 | 55.96 | |
| ATOM | 1649 | N9 | ADE | 77 | 46.342 | 26.527 | 8.686 | 1.00 | 54.59 | |
| ATOM | 1650 | C4 | ADE | 77 | 44.992 | 26.301 | 8.800 | 1.00 | 53.65 | |
| ATOM | 1651 | N3 | ADE | 77 | 44.048 | 26.566 | 7.892 | 1.00 | 54.88 | |
| ATOM | 1652 | C2 | ADE | 77 | 42.840 | 26.196 | 8.343 | 1.00 | 55.07 | |
| ATOM | 1653 | N1 | ADE | 77 | 42.508 | 25.637 | 9.512 | 1.00 | 54.24 | |
| ATOM | 1654 | C6 | ADE | 77 | 43.491 | 25.385 | 10.394 | 1.00 | 53.79 | |
| ATOM | 1655 | N6 | ADE | 77 | 43.164 | 24.825 | 11.537 | 1.00 | 55.70 | |
| ATOM | 1656 | C5 | ADE | 77 | 44.804 | 25.727 | 10.039 | 1.00 | 53.30 | |
| ATOM | 1657 | N7 | ADE | 77 | 46.011 | 25.607 | 10.710 | 1.00 | 54.75 | |
| ATOM | 1658 | C8 | ADE | 77 | 46.893 | 26.102 | 9.867 | 1.00 | 54.30 | |
| ATOM | 1659 | C2′ | ADE | 77 | 47.157 | 28.599 | 7.570 | 1.00 | 56.34 | |
| ATOM | 1660 | O2′ | ADE | 77 | 47.447 | 29.027 | 6.260 | 1.00 | 55.87 | |
| ATOM | 1661 | C3′ | ADE | 77 | 48.437 | 28.756 | 8.352 | 1.00 | 56.26 | |
| ATOM | 1662 | O3′ | ADE | 77 | 48.989 | 30.016 | 8.084 | 1.00 | 56.11 | |
| ATOM | 1663 | P | CYT | 78 | 49.363 | 30.973 | 9.304 | 1.00 | 56.18 | |
| ATOM | 1664 | O1P | CYT | 78 | 50.427 | 30.316 | 10.101 | 1.00 | 57.36 | |
| ATOM | 1665 | O2P | CYT | 78 | 49.611 | 32.316 | 8.727 | 1.00 | 54.95 | |
| ATOM | 1666 | O5′ | CYT | 78 | 48.044 | 30.952 | 10.194 | 1.00 | 54.79 | |
| ATOM | 1667 | C5′ | CYT | 78 | 46.964 | 31.806 | 9.887 | 1.00 | 53.51 | |
| ATOM | 1668 | C4′ | CYT | 78 | 45.682 | 31.260 | 10.453 | 1.00 | 52.70 | |
| ATOM | 1669 | O4′ | CYT | 78 | 45.756 | 29.813 | 10.463 | 1.00 | 53.28 | |
| ATOM | 1670 | C1′ | CYT | 78 | 45.106 | 29.301 | 11.608 | 1.00 | 50.29 | |
| ATOM | 1671 | N1 | CYT | 78 | 46.149 | 28.756 | 12.478 | 1.00 | 48.57 | |
| ATOM | 1672 | C6 | CYT | 78 | 47.464 | 29.027 | 12.241 | 1.00 | 47.31 | |
| ATOM | 1673 | C2 | CYT | 78 | 45.775 | 27.947 | 13.545 | 1.00 | 47.01 | |
| ATOM | 1674 | O2 | CYT | 78 | 44.578 | 27.760 | 13.738 | 1.00 | 46.27 | |
| ATOM | 1675 | N3 | CYT | 78 | 46.724 | 27.401 | 14.337 | 1.00 | 46.39 | |
| ATOM | 1676 | C4 | CYT | 78 | 48.011 | 27.651 | 14.090 | 1.00 | 47.75 | |
| ATOM | 1677 | N4 | CYT | 78 | 48.935 | 27.078 | 14.879 | 1.00 | 47.41 | |
| ATOM | 1678 | C5 | CYT | 78 | 48.420 | 28.500 | 13.013 | 1.00 | 48.62 | |
| ATOM | 1679 | C2′ | CYT | 78 | 44.421 | 30.480 | 12.275 | 1.00 | 52.24 | |
| ATOM | 1680 | O2′ | CYT | 78 | 43.149 | 30.633 | 11.667 | 1.00 | 53.32 | |
| ATOM | 1681 | C3′ | CYT | 78 | 45.370 | 31.602 | 11.888 | 1.00 | 52.36 | |
| ATOM | 1682 | O3′ | CYT | 78 | 44.767 | 32.867 | 11.972 | 1.00 | 52.96 | |
| ATOM | 1683 | P | GUA | 79 | 45.055 | 33.781 | 13.249 | 1.00 | 52.25 | |
| ATOM | 1684 | O1P | GUA | 79 | 46.463 | 33.556 | 13.687 | 1.00 | 53.81 | |
| ATOM | 1685 | O2P | GUA | 79 | 44.643 | 35.139 | 12.850 | 1.00 | 54.53 | |
| ATOM | 1686 | O5′ | GUA | 79 | 44.035 | 33.188 | 14.317 | 1.00 | 50.97 | |
| ATOM | 1687 | C5′ | GUA | 79 | 42.703 | 32.922 | 13.927 | 1.00 | 51.93 | |
| ATOM | 1688 | C4′ | GUA | 79 | 42.013 | 32.059 | 14.939 | 1.00 | 52.48 | |
| ATOM | 1689 | O4′ | GUA | 79 | 42.658 | 30.760 | 14.942 | 1.00 | 54.85 | |
| ATOM | 1690 | C1′ | GUA | 79 | 42.625 | 30.222 | 16.255 | 1.00 | 53.92 | |
| ATOM | 1691 | N9 | GUA | 79 | 43.974 | 29.859 | 16.686 | 1.00 | 50.75 | |
| ATOM | 1692 | C4 | GUA | 79 | 44.255 | 29.058 | 17.761 | 1.00 | 49.73 | |
| ATOM | 1693 | N3 | GUA | 79 | 43.348 | 28.483 | 18.555 | 1.00 | 50.01 | |
| ATOM | 1694 | C2 | GUA | 79 | 43.907 | 27.768 | 19.502 | 1.00 | 48.64 | |
| ATOM | 1695 | N2 | GUA | 79 | 43.115 | 27.131 | 20.374 | 1.00 | 52.64 | |
| ATOM | 1696 | N1 | GUA | 79 | 45.248 | 27.624 | 19.667 | 1.00 | 44.22 | |
| ATOM | 1697 | C6 | GUA | 79 | 46.212 | 28.212 | 18.867 | 1.00 | 46.79 | |
| ATOM | 1698 | O6 | GUA | 79 | 47.429 | 28.022 | 19.114 | 1.00 | 46.43 | |
| ATOM | 1699 | C5 | GUA | 79 | 45.623 | 28.987 | 17.829 | 1.00 | 47.92 | |
| ATOM | 1700 | N7 | GUA | 79 | 46.204 | 29.727 | 16.807 | 1.00 | 48.58 | |
| ATOM | 1701 | C8 | GUA | 79 | 45.183 | 30.227 | 16.152 | 1.00 | 49.85 | |
| ATOM | 1702 | C2′ | GUA | 79 | 41.945 | 31.261 | 17.149 | 1.00 | 54.76 | |
| ATOM | 1703 | O2′ | GUA | 79 | 40.553 | 31.024 | 17.166 | 1.00 | 57.98 | |
| ATOM | 1704 | C3′ | GUA | 79 | 42.190 | 32.529 | 16.366 | 1.00 | 54.12 | |
| ATOM | 1705 | O3′ | GUA | 79 | 41.254 | 33.519 | 16.725 | 1.00 | 55.51 | |
| ATOM | 1706 | P | URI | 80 | 41.739 | 34.747 | 17.638 | 1.00 | 55.85 | |
| ATOM | 1707 | O1P | URI | 80 | 40.670 | 35.757 | 17.569 | 1.00 | 55.96 | |
| ATOM | 1708 | O2P | URI | 80 | 43.122 | 35.105 | 17.248 | 1.00 | 53.77 | |
| ATOM | 1709 | O5′ | URI | 80 | 41.750 | 34.105 | 19.093 | 1.00 | 58.51 | |
| ATOM | 1710 | C5′ | URI | 80 | 40.581 | 33.443 | 19.578 | 1.00 | 59.91 | |
| ATOM | 1711 | C4′ | URI | 80 | 40.904 | 32.601 | 20.786 | 1.00 | 60.24 | |
| ATOM | 1712 | O4′ | URI | 80 | 41.720 | 31.463 | 20.400 | 1.00 | 60.36 | |
| ATOM | 1713 | C1′ | URI | 80 | 42.612 | 31.142 | 21.450 | 1.00 | 57.81 | |
| ATOM | 1714 | N1 | URI | 80 | 43.974 | 31.258 | 20.959 | 1.00 | 55.40 | |
| ATOM | 1715 | C6 | URI | 80 | 44.318 | 32.107 | 19.944 | 1.00 | 53.91 | |
| ATOM | 1716 | C2 | URI | 80 | 44.898 | 30.475 | 21.584 | 1.00 | 53.83 | |
| ATOM | 1717 | O2 | URI | 80 | 44.601 | 29.743 | 22.496 | 1.00 | 54.26 | |
| ATOM | 1718 | N3 | URI | 80 | 46.176 | 30.590 | 21.117 | 1.00 | 50.91 | |
| ATOM | 1719 | C4 | URI | 80 | 46.599 | 31.407 | 20.117 | 1.00 | 50.65 | |
| ATOM | 1720 | O4 | URI | 80 | 47.786 | 31.426 | 19.837 | 1.00 | 51.41 | |
| ATOM | 1721 | C5 | URI | 80 | 45.573 | 32.208 | 19.509 | 1.00 | 52.40 | |
| ATOM | 1722 | C2′ | URI | 80 | 42.402 | 32.151 | 22.563 | 1.00 | 59.90 | |
| ATOM | 1723 | O2′ | URI | 80 | 41.537 | 31.574 | 23.505 | 1.00 | 61.19 | |
| ATOM | 1724 | C3′ | URI | 80 | 41.775 | 33.307 | 21.800 | 1.00 | 61.84 | |
| ATOM | 1725 | O3′ | URI | 80 | 40.981 | 34.135 | 22.620 | 1.00 | 65.89 | |
| ATOM | 1726 | P | URI | 81 | 41.688 | 35.116 | 23.670 | 1.00 | 69.02 | |
| ATOM | 1727 | O1P | URI | 81 | 40.553 | 35.866 | 24.272 | 1.00 | 69.25 | |
| ATOM | 1728 | O2P | URI | 81 | 42.836 | 35.848 | 23.067 | 1.00 | 67.08 | |
| ATOM | 1729 | O5′ | URI | 81 | 42.298 | 34.137 | 24.766 | 1.00 | 68.61 | |
| ATOM | 1730 | C5′ | URI | 81 | 41.473 | 33.574 | 25.778 | 1.00 | 68.80 | |
| ATOM | 1731 | C4′ | URI | 81 | 42.327 | 32.807 | 26.752 | 1.00 | 69.26 | |
| ATOM | 1732 | O4′ | URI | 81 | 43.045 | 31.785 | 26.020 | 1.00 | 69.66 | |
| ATOM | 1733 | C1′ | URI | 81 | 44.332 | 31.612 | 26.584 | 1.00 | 68.52 | |
| ATOM | 1734 | N1 | URI | 81 | 45.344 | 31.952 | 25.589 | 1.00 | 66.30 | |
| ATOM | 1735 | C6 | URI | 81 | 45.093 | 32.756 | 24.508 | 1.00 | 66.26 | |
| ATOM | 1736 | C2 | URI | 81 | 46.570 | 31.439 | 25.810 | 1.00 | 65.61 | |
| ATOM | 1737 | O2 | URI | 81 | 46.802 | 30.741 | 26.772 | 1.00 | 66.73 | |
| ATOM | 1738 | N3 | URI | 81 | 47.525 | 31.775 | 24.886 | 1.00 | 63.48 | |
| ATOM | 1739 | C4 | URI | 81 | 47.367 | 32.570 | 23.801 | 1.00 | 61.53 | |
| ATOM | 1740 | O4 | URI | 81 | 48.322 | 32.780 | 23.082 | 1.00 | 60.99 | |
| ATOM | 1741 | C5 | URI | 81 | 46.048 | 33.081 | 23.625 | 1.00 | 64.25 | |
| ATOM | 1742 | C2′ | URI | 81 | 44.459 | 32.566 | 27.765 | 1.00 | 69.95 | |
| ATOM | 1743 | O2′ | URI | 81 | 44.201 | 31.895 | 28.980 | 1.00 | 71.21 | |
| ATOM | 1744 | C3′ | URI | 81 | 43.437 | 33.627 | 27.389 | 1.00 | 70.46 | |
| ATOM | 1745 | O3′ | URI | 81 | 42.987 | 34.354 | 28.522 | 1.00 | 72.50 | |
| ATOM | 1746 | P | GUA | 82 | 43.822 | 35.626 | 29.035 | 1.00 | 73.86 | |
| ATOM | 1747 | O1P | GUA | 82 | 44.150 | 36.503 | 27.883 | 1.00 | 75.42 | |
| ATOM | 1748 | O2P | GUA | 82 | 42.997 | 36.165 | 30.130 | 1.00 | 76.05 | |
| ATOM | 1749 | O5′ | GUA | 82 | 45.167 | 35.004 | 29.634 | 1.00 | 72.13 | |
| ATOM | 1750 | C5′ | GUA | 82 | 45.091 | 34.076 | 30.713 | 1.00 | 72.12 | |
| ATOM | 1751 | C4′ | GUA | 82 | 46.454 | 33.510 | 31.053 | 1.00 | 72.41 | |
| ATOM | 1752 | O4′ | GUA | 82 | 46.972 | 32.715 | 29.948 | 1.00 | 71.27 | |
| ATOM | 1753 | C1′ | GUA | 82 | 48.383 | 32.815 | 29.918 | 1.00 | 69.55 | |
| ATOM | 1754 | N9 | GUA | 82 | 48.807 | 33.207 | 28.585 | 1.00 | 68.11 | |
| ATOM | 1755 | C4 | GUA | 82 | 50.035 | 32.951 | 27.998 | 1.00 | 65.75 | |
| ATOM | 1756 | N3 | GUA | 82 | 51.050 | 32.248 | 28.542 | 1.00 | 64.37 | |
| ATOM | 1757 | C2 | GUA | 82 | 52.097 | 32.209 | 27.738 | 1.00 | 64.74 | |
| ATOM | 1758 | N2 | GUA | 82 | 53.207 | 31.553 | 28.101 | 1.00 | 65.43 | |
| ATOM | 1759 | N1 | GUA | 82 | 52.141 | 32.811 | 26.512 | 1.00 | 63.80 | |
| ATOM | 1760 | C6 | GUA | 82 | 51.104 | 33.530 | 25.946 | 1.00 | 63.80 | |
| ATOM | 1761 | O6 | GUA | 82 | 51.244 | 34.037 | 24.833 | 1.00 | 65.67 | |
| ATOM | 1762 | C5 | GUA | 82 | 49.988 | 33.574 | 26.781 | 1.00 | 64.12 | |
| ATOM | 1763 | N7 | GUA | 82 | 48.756 | 34.179 | 26.585 | 1.00 | 66.32 | |
| ATOM | 1764 | C8 | GUA | 82 | 48.086 | 33.929 | 27.677 | 1.00 | 66.82 | |
| ATOM | 1765 | C2′ | GUA | 82 | 48.808 | 33.809 | 30.993 | 1.00 | 70.32 | |
| ATOM | 1766 | O2′ | GUA | 82 | 49.261 | 33.147 | 32.142 | 1.00 | 70.43 | |
| ATOM | 1767 | O3′ | GUA | 82 | 47.518 | 34.571 | 31.246 | 1.00 | 72.50 | |
| ATOM | 1768 | C3′ | GUA | 82 | 47.487 | 35.070 | 32.578 | 1.00 | 73.64 | |
| ATOM | 1769 | P | ADE | 83 | 48.574 | 36.151 | 33.031 | 1.00 | 75.34 | |
| ATOM | 1770 | O1P | ADE | 83 | 48.599 | 37.236 | 31.999 | 1.00 | 75.09 | |
| ATOM | 1771 | O2P | ADE | 83 | 48.310 | 36.491 | 34.453 | 1.00 | 76.21 | |
| ATOM | 1772 | O5′ | ADE | 83 | 49.954 | 35.355 | 32.955 | 1.00 | 72.44 | |
| ATOM | 1773 | C5′ | ADE | 83 | 51.176 | 36.050 | 32.898 | 1.00 | 70.52 | |
| ATOM | 1774 | C4′ | ADE | 83 | 52.288 | 35.132 | 32.458 | 1.00 | 71.38 | |
| ATOM | 1775 | O4′ | ADE | 83 | 51.956 | 34.480 | 31.201 | 1.00 | 70.99 | |
| ATOM | 1776 | C1′ | ADE | 83 | 53.132 | 34.356 | 30.409 | 1.00 | 69.22 | |
| ATOM | 1777 | N9 | ADE | 83 | 52.928 | 35.064 | 29.148 | 1.00 | 66.75 | |
| ATOM | 1778 | C4 | ADE | 83 | 53.887 | 35.197 | 28.174 | 1.00 | 64.40 | |
| ATOM | 1779 | N3 | ADE | 83 | 55.143 | 34.718 | 28.202 | 1.00 | 63.01 | |
| ATOM | 1780 | C2 | ADE | 83 | 55.784 | 35.031 | 27.090 | 1.00 | 62.65 | |
| ATOM | 1781 | N1 | ADE | 83 | 55.347 | 35.718 | 26.031 | 1.00 | 63.95 | |
| ATOM | 1782 | C6 | ADE | 83 | 54.078 | 36.191 | 26.043 | 1.00 | 64.20 | |
| ATOM | 1783 | N6 | ADE | 83 | 53.636 | 36.884 | 24.991 | 1.00 | 64.73 | |
| ATOM | 1784 | C5 | ADE | 83 | 53.296 | 35.925 | 27.164 | 1.00 | 63.85 | |
| ATOM | 1785 | N7 | ADE | 83 | 51.987 | 36.253 | 27.490 | 1.00 | 64.21 | |
| ATOM | 1786 | C8 | ADE | 83 | 51.815 | 35.718 | 28.675 | 1.00 | 65.25 | |
| ATOM | 1787 | C2′ | ADE | 83 | 54.295 | 34.978 | 31.185 | 1.00 | 70.57 | |
| ATOM | 1788 | O2′ | ADE | 83 | 55.115 | 34.014 | 31.805 | 1.00 | 71.46 | |
| ATOM | 1789 | C3′ | ADE | 83 | 53.559 | 35.895 | 32.144 | 1.00 | 71.71 | |
| ATOM | 1790 | O3′ | ADE | 83 | 54.284 | 36.075 | 33.334 | 1.00 | 73.71 | |
| ATOM | 1791 | P | ADE | 84 | 55.215 | 37.356 | 33.492 | 1.00 | 75.40 | |
| ATOM | 1792 | O1P | ADE | 84 | 54.328 | 38.543 | 33.307 | 1.00 | 73.29 | |
| ATOM | 1793 | O2P | ADE | 84 | 55.951 | 37.177 | 34.783 | 1.00 | 76.08 | |
| ATOM | 1794 | O5′ | ADE | 84 | 56.245 | 37.207 | 32.277 | 1.00 | 72.22 | |
| ATOM | 1795 | C5′ | ADE | 84 | 57.266 | 36.212 | 32.305 | 1.00 | 70.53 | |
| ATOM | 1796 | C4′ | ADE | 84 | 58.240 | 36.441 | 31.174 | 1.00 | 69.60 | |
| ATOM | 1797 | O4′ | ADE | 84 | 57.673 | 35.976 | 29.932 | 1.00 | 68.64 | |
| ATOM | 1798 | C1′ | ADE | 84 | 58.025 | 36.864 | 28.891 | 1.00 | 66.28 | |
| ATOM | 1799 | N9 | ADE | 84 | 56.809 | 37.578 | 28.538 | 1.00 | 62.60 | |
| ATOM | 1800 | C4 | ADE | 84 | 56.596 | 38.297 | 27.393 | 1.00 | 59.58 | |
| ATOM | 1801 | N3 | ADE | 84 | 57.456 | 38.469 | 26.378 | 1.00 | 59.08 | |
| ATOM | 1802 | C2 | ADE | 84 | 56.907 | 39.214 | 25.437 | 1.00 | 58.05 | |
| ATOM | 1803 | N1 | ADE | 84 | 55.689 | 39.767 | 25.402 | 1.00 | 57.22 | |
| ATOM | 1804 | C6 | ADE | 84 | 54.860 | 39.572 | 26.437 | 1.00 | 56.28 | |
| ATOM | 1805 | N6 | ADE | 84 | 53.654 | 40.125 | 26.387 | 1.00 | 56.50 | |
| ATOM | 1806 | C5 | ADE | 84 | 55.322 | 38.801 | 27.498 | 1.00 | 57.61 | |
| ATOM | 1807 | N7 | ADE | 84 | 54.741 | 38.416 | 28.698 | 1.00 | 59.83 | |
| ATOM | 1808 | C8 | ADE | 84 | 55.661 | 37.688 | 29.273 | 1.00 | 60.69 | |
| ATOM | 1809 | C2′ | ADE | 84 | 59.040 | 37.854 | 29.457 | 1.00 | 68.38 | |
| ATOM | 1810 | O2′ | ADE | 84 | 60.371 | 37.436 | 29.227 | 1.00 | 69.05 | |
| ATOM | 1811 | C3′ | ADE | 84 | 58.625 | 37.882 | 30.918 | 1.00 | 69.64 | |
| ATOM | 1812 | O3′ | ADE | 84 | 59.694 | 38.236 | 31.761 | 1.00 | 72.21 | |
| ATOM | 1813 | P | ADE | 85 | 60.055 | 39.778 | 31.975 | 1.00 | 73.21 | |
| ATOM | 1814 | O1P | ADE | 85 | 58.953 | 40.467 | 32.711 | 1.00 | 72.97 | |
| ATOM | 1815 | O2P | ADE | 85 | 61.446 | 39.796 | 32.522 | 1.00 | 74.91 | |
| ATOM | 1816 | O5′ | ADE | 85 | 60.106 | 40.355 | 30.500 | 1.00 | 70.60 | |
| ATOM | 1817 | C5′ | ADE | 85 | 61.274 | 40.229 | 29.720 | 1.00 | 66.97 | |
| ATOM | 1818 | C4′ | ADE | 85 | 61.116 | 41.046 | 28.465 | 1.00 | 66.53 | |
| ATOM | 1819 | O4′ | ADE | 85 | 59.958 | 40.555 | 27.713 | 1.00 | 64.13 | |
| ATOM | 1820 | C1′ | ADE | 85 | 59.293 | 41.639 | 27.100 | 1.00 | 59.44 | |
| ATOM | 1821 | N9 | ADE | 85 | 57.997 | 41.776 | 27.755 | 1.00 | 55.49 | |
| ATOM | 1822 | C4 | ADE | 85 | 56.900 | 42.446 | 27.263 | 1.00 | 52.55 | |
| ATOM | 1823 | N3 | ADE | 85 | 56.804 | 43.102 | 26.094 | 1.00 | 51.73 | |
| ATOM | 1824 | C2 | ADE | 85 | 55.612 | 43.626 | 25.952 | 1.00 | 49.86 | |
| ATOM | 1825 | N1 | ADE | 85 | 54.574 | 43.578 | 26.770 | 1.00 | 50.86 | |
| ATOM | 1826 | C6 | ADE | 85 | 54.689 | 42.902 | 27.927 | 1.00 | 50.76 | |
| ATOM | 1827 | N6 | ADE | 85 | 53.624 | 42.843 | 28.735 | 1.00 | 49.94 | |
| ATOM | 1828 | C5 | ADE | 85 | 55.918 | 42.301 | 28.205 | 1.00 | 50.46 | |
| ATOM | 1829 | N7 | ADE | 85 | 56.374 | 41.549 | 29.277 | 1.00 | 52.22 | |
| ATOM | 1830 | C5 | ADE | 85 | 57.613 | 41.267 | 28.962 | 1.00 | 53.39 | |
| ATOM | 1831 | C2′ | ADE | 85 | 60.141 | 42.877 | 27.360 | 1.00 | 62.75 | |
| ATOM | 1832 | O2′ | ADE | 85 | 61.142 | 42.989 | 26.361 | 1.00 | 63.18 | |
| ATOM | 1833 | C3′ | ADE | 85 | 60.781 | 42.508 | 28.685 | 1.00 | 64.50 | |
| ATOM | 1834 | O3′ | ADE | 85 | 61.914 | 43.304 | 28.944 | 1.00 | 65.50 | |
| ATOM | 1835 | P | GUA | 86 | 61.734 | 44.672 | 29.761 | 1.00 | 65.91 | |
| ATOM | 1836 | O1P | GUA | 86 | 60.991 | 44.326 | 31.009 | 1.00 | 64.38 | |
| ATOM | 1837 | O2P | GUA | 86 | 63.074 | 45.312 | 29.819 | 1.00 | 65.95 | |
| ATOM | 1838 | O5′ | GUA | 86 | 60.813 | 45.582 | 28.835 | 1.00 | 63.79 | |
| ATOM | 1839 | C5′ | GUA | 86 | 61.313 | 46.022 | 27.588 | 1.00 | 59.94 | |
| ATOM | 1840 | C4′ | GUA | 86 | 60.239 | 46.700 | 26.765 | 1.00 | 57.11 | |
| ATOM | 1841 | O4′ | GUA | 86 | 59.063 | 45.841 | 26.677 | 1.00 | 55.61 | |
| ATOM | 1842 | C1′ | GUA | 86 | 57.893 | 46.643 | 26.583 | 1.00 | 52.76 | |
| ATOM | 1843 | N9 | GUA | 86 | 56.989 | 46.244 | 27.643 | 1.00 | 50.33 | |
| ATOM | 1844 | C4 | GUA | 86 | 55.706 | 46.699 | 27.862 | 1.00 | 49.46 | |
| ATOM | 1845 | N3 | GUA | 86 | 55.051 | 47.642 | 27.144 | 1.00 | 48.57 | |
| ATOM | 1846 | C2 | GUA | 86 | 53.809 | 47.831 | 27.603 | 1.00 | 48.63 | |
| ATOM | 1847 | N2 | GUA | 86 | 52.995 | 48.731 | 27.055 | 1.00 | 47.89 | |
| ATOM | 1848 | N1 | GUA | 86 | 53.267 | 47.149 | 28.650 | 1.00 | 50.62 | |
| ATOM | 1849 | C6 | GUA | 86 | 53.929 | 46.186 | 29.397 | 1.00 | 50.83 | |
| ATOM | 1850 | O6 | GUA | 86 | 53.347 | 45.636 | 30.326 | 1.00 | 53.41 | |
| ATOM | 1851 | C5 | GUA | 86 | 55.247 | 45.977 | 28.940 | 1.00 | 49.00 | |
| ATOM | 1852 | N7 | GUA | 86 | 56.224 | 45.112 | 29.405 | 1.00 | 49.65 | |
| ATOM | 1853 | C8 | GUA | 86 | 57.242 | 45.309 | 28.609 | 1.00 | 49.95 | |
| ATOM | 1854 | C2′ | GUA | 86 | 58.331 | 48.102 | 26.659 | 1.00 | 54.64 | |
| ATOM | 1855 | O2′ | GUA | 86 | 58.496 | 48.591 | 25.342 | 1.00 | 53.47 | |
| ATOM | 1856 | C3′ | GUA | 86 | 59.668 | 47.966 | 27.375 | 1.00 | 55.15 | |
| ATOM | 1857 | O3′ | GUA | 86 | 60.524 | 49.074 | 27.209 | 1.00 | 53.47 | |
| ATOM | 1858 | P | ADE | 87 | 60.507 | 50.233 | 28.312 | 1.00 | 50.30 | |
| ATOM | 1859 | O1P | ADE | 87 | 60.524 | 49.594 | 29.626 | 1.00 | 52.25 | |
| ATOM | 1860 | O2P | ADE | 87 | 61.527 | 51.229 | 27.973 | 1.00 | 53.20 | |
| ATOM | 1861 | O5′ | ADE | 87 | 59.103 | 50.927 | 28.069 | 1.00 | 49.73 | |
| ATOM | 1862 | C5′ | ADE | 87 | 58.937 | 51.804 | 26.969 | 1.00 | 46.19 | |
| ATOM | 1863 | C4′ | ADE | 87 | 57.523 | 52.302 | 26.908 | 1.00 | 44.70 | |
| ATOM | 1864 | O4′ | ADE | 87 | 56.653 | 51.165 | 27.051 | 1.00 | 43.85 | |
| ATOM | 1865 | C1′ | ADE | 87 | 55.473 | 51.539 | 27.742 | 1.00 | 42.06 | |
| ATOM | 1866 | N9 | ADE | 87 | 55.325 | 50.642 | 28.866 | 1.00 | 39.42 | |
| ATOM | 1867 | C4 | ADE | 87 | 54.185 | 50.441 | 29.593 | 1.00 | 38.57 | |
| ATOM | 1868 | N3 | ADE | 87 | 53.013 | 51.065 | 29.438 | 1.00 | 39.50 | |
| ATOM | 1869 | C2 | ADE | 87 | 52.122 | 50.598 | 30.308 | 1.00 | 39.89 | |
| ATOM | 1870 | N1 | ADE | 87 | 52.267 | 49.642 | 31.235 | 1.00 | 39.74 | |
| ATOM | 1871 | C6 | ADE | 87 | 53.460 | 49.032 | 31.343 | 1.00 | 37.28 | |
| ATOM | 1872 | N6 | ADE | 87 | 53.591 | 48.076 | 32.239 | 1.00 | 37.66 | |
| ATOM | 1873 | C5 | ADE | 87 | 54.480 | 49.446 | 30.496 | 1.00 | 37.04 | |
| ATOM | 1874 | N7 | ADE | 87 | 55.792 | 49.039 | 30.354 | 1.00 | 40.30 | |
| ATOM | 1875 | C8 | ADE | 87 | 56.255 | 49.791 | 29.380 | 1.00 | 39.46 | |
| ATOM | 1876 | C2′ | ADE | 87 | 55.610 | 52.997 | 28.129 | 1.00 | 45.04 | |
| ATOM | 1877 | O2′ | ADE | 87 | 54.977 | 53.803 | 27.158 | 1.00 | 47.02 | |
| ATOM | 1878 | C3′ | ADE | 87 | 57.119 | 53.140 | 28.103 | 1.00 | 47.03 | |
| ATOM | 1879 | O3′ | ADE | 87 | 57.556 | 54.471 | 27.997 | 1.00 | 48.62 | |
| ATOM | 1880 | P | URI | 88 | 58.202 | 55.161 | 29.279 | 1.00 | 48.31 | |
| ATOM | 1881 | O1P | URI | 88 | 58.842 | 54.087 | 30.082 | 1.00 | 47.18 | |
| ATOM | 1882 | O2P | URI | 88 | 59.028 | 56.264 | 28.718 | 1.00 | 48.10 | |
| ATOM | 1883 | O5′ | URI | 88 | 56.903 | 55.702 | 30.033 | 1.00 | 47.72 | |
| ATOM | 1884 | C5′ | URI | 88 | 56.114 | 56.709 | 29.417 | 1.00 | 48.06 | |
| ATOM | 1885 | C4′ | URI | 88 | 54.832 | 56.909 | 30.168 | 1.00 | 48.17 | |
| ATOM | 1886 | O4′ | URI | 88 | 54.061 | 55.688 | 30.086 | 1.00 | 48.56 | |
| ATOM | 1887 | C1′ | URI | 88 | 53.342 | 55.492 | 31.285 | 1.00 | 46.52 | |
| ATOM | 1888 | N1 | URI | 88 | 53.832 | 54.260 | 31.899 | 1.00 | 44.89 | |
| ATOM | 1889 | C6 | URI | 88 | 54.977 | 53.660 | 31.480 | 1.00 | 44.74 | |
| ATOM | 1890 | C2 | URI | 88 | 53.093 | 53.741 | 32.918 | 1.00 | 45.35 | |
| ATOM | 1891 | O2 | URI | 88 | 52.068 | 54.262 | 33.292 | 1.00 | 48.00 | |
| ATOM | 1892 | N3 | URI | 88 | 53.588 | 52.598 | 33.487 | 1.00 | 43.30 | |
| ATOM | 1893 | C4 | URI | 88 | 54.727 | 51.943 | 33.127 | 1.00 | 44.05 | |
| ATOM | 1894 | O4 | URI | 88 | 55.064 | 50.932 | 33.743 | 1.00 | 46.21 | |
| ATOM | 1895 | C5 | URI | 88 | 55.442 | 52.541 | 32.040 | 1.00 | 44.32 | |
| ATOM | 1896 | C2′ | URI | 88 | 53.616 | 56.705 | 32.164 | 1.00 | 47.51 | |
| ATOM | 1897 | O2′ | URI | 88 | 52.731 | 57.724 | 31.775 | 1.00 | 48.89 | |
| ATOM | 1898 | C3′ | URI | 88 | 54.984 | 57.111 | 31.661 | 1.00 | 48.60 | |
| ATOM | 1899 | O3′ | URI | 88 | 55.292 | 58.445 | 32.005 | 1.00 | 48.21 | |
| ATOM | 1900 | P | GUA | 89 | 56.099 | 58.726 | 33.360 | 1.00 | 46.62 | |
| ATOM | 1901 | O1P | GUA | 89 | 57.054 | 57.603 | 33.470 | 1.00 | 43.08 | |
| ATOM | 1902 | O2P | GUA | 89 | 56.593 | 60.121 | 33.289 | 1.00 | 46.71 | |
| ATOM | 1903 | O5′ | GUA | 89 | 54.981 | 58.579 | 34.492 | 1.00 | 46.33 | |
| ATOM | 1904 | C5′ | GUA | 89 | 53.812 | 59.384 | 34.464 | 1.00 | 46.68 | |
| ATOM | 1905 | C4′ | GUA | 89 | 52.835 | 58.957 | 35.540 | 1.00 | 47.99 | |
| ATOM | 1906 | O4′ | GUA | 89 | 52.428 | 57.576 | 35.329 | 1.00 | 48.45 | |
| ATOM | 1907 | C1′ | GUA | 89 | 52.197 | 56.947 | 36.589 | 1.00 | 45.89 | |
| ATOM | 1908 | N9 | GUA | 89 | 53.032 | 55.754 | 36.678 | 1.00 | 42.60 | |
| ATOM | 1909 | C4 | GUA | 89 | 52.803 | 54.636 | 37.449 | 1.00 | 39.75 | |
| ATOM | 1910 | N3 | GUA | 89 | 51.793 | 54.464 | 38.301 | 1.00 | 41.19 | |
| ATOM | 1911 | C2 | GUA | 89 | 51.842 | 53.263 | 38.883 | 1.00 | 41.82 | |
| ATOM | 1912 | N2 | GUA | 89 | 50.904 | 52.900 | 39.752 | 1.00 | 45.31 | |
| ATOM | 1913 | N1 | GUA | 89 | 52.803 | 52.330 | 38.658 | 1.00 | 38.19 | |
| ATOM | 1914 | C6 | GUA | 89 | 53.852 | 52.493 | 37.790 | 1.00 | 38.45 | |
| ATOM | 1915 | O6 | GUA | 89 | 54.667 | 51.579 | 37.661 | 1.00 | 37.98 | |
| ATOM | 1916 | C5 | GUA | 89 | 53.816 | 53.764 | 37.148 | 1.00 | 38.40 | |
| ATOM | 1917 | N7 | GUA | 89 | 54.682 | 54.326 | 36.228 | 1.00 | 41.24 | |
| ATOM | 1918 | C8 | GUA | 89 | 54.183 | 55.510 | 35.984 | 1.00 | 42.16 | |
| ATOM | 1919 | C2′ | GUA | 89 | 52.451 | 57.991 | 37.674 | 1.00 | 47.11 | |
| ATOM | 1920 | O2′ | GUA | 89 | 51.227 | 58.632 | 38.023 | 1.00 | 44.62 | |
| ATOM | 1921 | C3′ | GUA | 89 | 53.394 | 58.942 | 36.955 | 1.00 | 48.11 | |
| ATOM | 1922 | O3′ | GUA | 89 | 53.321 | 60.237 | 37.514 | 1.00 | 50.91 | |
| ATOM | 1923 | P | ADE | 90 | 54.287 | 60.635 | 38.725 | 1.00 | 51.44 | |
| ATOM | 1924 | O1P | ADE | 90 | 55.686 | 60.461 | 38.300 | 1.00 | 52.67 | |
| ATOM | 1925 | O2P | ADE | 90 | 53.807 | 61.982 | 39.098 | 1.00 | 53.59 | |
| ATOM | 1926 | O5′ | ADE | 90 | 53.906 | 59.593 | 39.871 | 1.00 | 50.44 | |
| ATOM | 1927 | C5′ | ADE | 90 | 52.759 | 59.848 | 40.687 | 1.00 | 49.68 | |
| ATOM | 1928 | C4′ | ADE | 90 | 52.605 | 58.790 | 41.742 | 1.00 | 49.60 | |
| ATOM | 1929 | O4′ | ADE | 90 | 52.416 | 57.527 | 41.087 | 1.00 | 48.50 | |
| ATOM | 1930 | C1′ | ADE | 90 | 53.027 | 56.504 | 41.831 | 1.00 | 46.22 | |
| ATOM | 1931 | N9 | ADE | 90 | 54.083 | 55.930 | 41.016 | 1.00 | 42.57 | |
| ATOM | 1932 | C4 | ADE | 90 | 54.525 | 54.647 | 41.137 | 1.00 | 39.21 | |
| ATOM | 1933 | N3 | ADE | 90 | 54.075 | 53.742 | 41.992 | 1.00 | 39.75 | |
| ATOM | 1934 | C2 | ADE | 90 | 54.743 | 52.614 | 41.857 | 1.00 | 41.35 | |
| ATOM | 1935 | N1 | ADE | 90 | 55.733 | 52.320 | 41.024 | 1.00 | 40.92 | |
| ATOM | 1936 | C6 | ADE | 90 | 56.151 | 53.270 | 40.172 | 1.00 | 40.07 | |
| ATOM | 1937 | N6 | ADE | 90 | 57.135 | 52.976 | 39.340 | 1.00 | 41.92 | |
| ATOM | 1938 | C5 | ADE | 90 | 55.527 | 54.497 | 40.220 | 1.00 | 37.41 | |
| ATOM | 1939 | N7 | ADE | 90 | 55.724 | 55.660 | 39.515 | 1.00 | 39.02 | |
| ATOM | 1940 | C8 | ADE | 90 | 54.837 | 56.492 | 40.024 | 1.00 | 41.35 | |
| ATOM | 1941 | C2′ | ADE | 90 | 53.579 | 57.137 | 43.095 | 1.00 | 50.09 | |
| ATOM | 1942 | O2′ | ADE | 90 | 52.574 | 57.092 | 44.090 | 1.00 | 53.88 | |
| ATOM | 1943 | C3′ | ADE | 90 | 53.817 | 58.550 | 42.614 | 1.00 | 51.43 | |
| ATOM | 1944 | O3′ | ADE | 90 | 53.844 | 59.463 | 43.690 | 1.00 | 55.88 | |
| ATOM | 1945 | P | GUA | 91 | 55.249 | 59.891 | 44.330 | 1.00 | 58.76 | |
| ATOM | 1946 | O1P | GUA | 91 | 56.324 | 60.018 | 43.305 | 1.00 | 57.54 | |
| ATOM | 1947 | O2P | GUA | 91 | 54.901 | 61.084 | 45.161 | 1.00 | 59.32 | |
| ATOM | 1948 | O5′ | GUA | 91 | 55.587 | 58.606 | 45.211 | 1.00 | 56.24 | |
| ATOM | 1949 | C5′ | GUA | 91 | 54.654 | 58.130 | 46.147 | 1.00 | 55.27 | |
| ATOM | 1950 | C4′ | GUA | 91 | 55.108 | 56.813 | 46.705 | 1.00 | 57.42 | |
| ATOM | 1951 | O4′ | GUA | 91 | 54.952 | 55.779 | 45.701 | 1.00 | 57.61 | |
| ATOM | 1952 | C1′ | GUA | 91 | 55.996 | 54.822 | 45.842 | 1.00 | 55.92 | |
| ATOM | 1953 | N9 | GUA | 91 | 56.783 | 54.766 | 44.621 | 1.00 | 50.86 | |
| ATOM | 1954 | C4 | GUA | 91 | 57.531 | 53.698 | 44.210 | 1.00 | 47.48 | |
| ATOM | 1955 | N3 | GUA | 91 | 57.643 | 52.518 | 44.853 | 1.00 | 45.65 | |
| ATOM | 1956 | C2 | GUA | 91 | 58.458 | 51.684 | 44.217 | 1.00 | 45.38 | |
| ATOM | 1957 | N2 | GUA | 91 | 58.697 | 50.459 | 44.712 | 1.00 | 43.63 | |
| ATOM | 1958 | N1 | GUA | 91 | 59.095 | 51.986 | 43.053 | 1.00 | 45.16 | |
| ATOM | 1959 | C6 | GUA | 91 | 58.974 | 53.193 | 42.372 | 1.00 | 47.02 | |
| ATOM | 1960 | O6 | GUA | 91 | 59.569 | 53.361 | 41.302 | 1.00 | 48.12 | |
| ATOM | 1961 | C5 | GUA | 91 | 58.123 | 54.095 | 43.046 | 1.00 | 46.49 | |
| ATOM | 1962 | N7 | GUA | 91 | 57.747 | 55.390 | 42.716 | 1.00 | 47.05 | |
| ATOM | 1963 | C8 | GUA | 91 | 56.948 | 55.748 | 43.680 | 1.00 | 48.36 | |
| ATOM | 1964 | C2′ | GUA | 91 | 56.861 | 55.264 | 47.009 | 1.00 | 57.24 | |
| ATOM | 1965 | O2′ | GUA | 91 | 56.321 | 54.656 | 48.149 | 1.00 | 61.28 | |
| ATOM | 1966 | C3′ | GUA | 91 | 56.585 | 56.752 | 47.031 | 1.00 | 58.70 | |
| ATOM | 1967 | O3′ | GUA | 91 | 56.869 | 57.300 | 48.297 | 1.00 | 61.96 | |
| ATOM | 1968 | P | CYT | 92 | 58.404 | 57.505 | 48.726 | 1.00 | 66.23 | |
| ATOM | 1969 | O1P | CYT | 92 | 59.060 | 58.388 | 47.712 | 1.00 | 65.98 | |
| ATOM | 1970 | O2P | CYT | 92 | 58.486 | 57.865 | 50.170 | 1.00 | 64.76 | |
| ATOM | 1971 | O5′ | CYT | 92 | 58.979 | 56.040 | 48.553 | 1.00 | 61.89 | |
| ATOM | 1972 | C5′ | CYT | 92 | 60.213 | 55.707 | 49.107 | 1.00 | 58.84 | |
| ATOM | 1973 | C4′ | CYT | 92 | 60.482 | 54.271 | 48.863 | 1.00 | 56.69 | |
| ATOM | 1974 | O4′ | CYT | 92 | 59.823 | 53.883 | 47.652 | 1.00 | 56.03 | |
| ATOM | 1975 | C1′ | CYT | 92 | 60.657 | 52.995 | 46.932 | 1.00 | 53.96 | |
| ATOM | 1976 | N1 | CYT | 92 | 61.064 | 53.674 | 45.704 | 1.00 | 51.64 | |
| ATOM | 1977 | C6 | CYT | 92 | 60.786 | 54.990 | 45.500 | 1.00 | 51.38 | |
| ATOM | 1978 | C2 | CYT | 92 | 61.729 | 52.950 | 44.760 | 1.00 | 51.67 | |
| ATOM | 1979 | O2 | CYT | 92 | 61.953 | 51.762 | 44.983 | 1.00 | 54.04 | |
| ATOM | 1980 | N3 | CYT | 92 | 62.119 | 53.537 | 43.626 | 1.00 | 51.70 | |
| ATOM | 1981 | C4 | CYT | 92 | 61.842 | 54.814 | 43.420 | 1.00 | 51.10 | |
| ATOM | 1982 | N4 | CYT | 92 | 62.243 | 55.346 | 42.283 | 1.00 | 51.69 | |
| ATOM | 1983 | C5 | CYT | 92 | 61.145 | 55.592 | 44.375 | 1.00 | 51.24 | |
| ATOM | 1984 | C2′ | CYT | 92 | 61.863 | 52.692 | 47.811 | 1.00 | 54.28 | |
| ATOM | 1985 | O2′ | CYT | 92 | 61.568 | 51.559 | 48.588 | 1.00 | 54.76 | |
| ATOM | 1986 | C3′ | CYT | 92 | 61.933 | 53.965 | 48.629 | 1.00 | 55.63 | |
| ATOM | 1987 | O3′ | CYT | 92 | 62.585 | 53.839 | 49.868 | 1.00 | 56.30 | |
| ATOM | 1988 | P | CYT | 93 | 63.981 | 54.580 | 50.073 | 1.00 | 56.73 | |
| ATOM | 1989 | O1P | CYT | 93 | 63.923 | 55.872 | 49.347 | 1.00 | 55.93 | |
| ATOM | 1990 | O2P | CYT | 93 | 64.249 | 54.575 | 51.523 | 1.00 | 58.16 | |
| ATOM | 1991 | O5′ | CYT | 93 | 65.006 | 53.595 | 49.360 | 1.00 | 55.12 | |
| ATOM | 1992 | C5′ | CYT | 93 | 65.172 | 52.279 | 49.873 | 1.00 | 56.61 | |
| ATOM | 1993 | C4′ | CYT | 93 | 66.005 | 51.466 | 48.926 | 1.00 | 58.55 | |
| ATOM | 1994 | O4′ | CYT | 93 | 65.358 | 51.441 | 47.624 | 1.00 | 57.23 | |
| ATOM | 1995 | C1′ | CYT | 93 | 66.341 | 51.453 | 46.601 | 1.00 | 54.85 | |
| ATOM | 1996 | N1 | CYT | 93 | 66.078 | 52.594 | 45.719 | 1.00 | 50.99 | |
| ATOM | 1997 | C6 | CYT | 93 | 65.354 | 53.668 | 46.156 | 1.00 | 49.07 | |
| ATOM | 1998 | C2 | CYT | 93 | 66.548 | 52.547 | 44.411 | 1.00 | 48.99 | |
| ATOM | 1999 | O2 | CYT | 93 | 67.243 | 51.583 | 44.060 | 1.00 | 48.91 | |
| ATOM | 2000 | N3 | CYT | 93 | 66.238 | 53.548 | 43.565 | 1.00 | 46.69 | |
| ATOM | 2001 | C4 | CYT | 93 | 65.501 | 54.577 | 43.990 | 1.00 | 45.84 | |
| ATOM | 2002 | N4 | CYT | 93 | 65.185 | 55.530 | 43.108 | 1.00 | 47.85 | |
| ATOM | 2003 | C5 | CYT | 93 | 65.046 | 54.673 | 45.325 | 1.00 | 46.14 | |
| ATOM | 2004 | C2′ | CYT | 93 | 67.705 | 51.481 | 47.274 | 1.00 | 57.26 | |
| ATOM | 2005 | O2′ | CYT | 93 | 68.148 | 50.159 | 47.411 | 1.00 | 57.46 | |
| ATOM | 2006 | C3′ | CYT | 93 | 67.357 | 52.080 | 48.630 | 1.00 | 59.82 | |
| ATOM | 2007 | O3′ | CYT | 93 | 68.308 | 51.705 | 49.612 | 1.00 | 62.69 | |
| ATOM | 2008 | P | ADE | 94 | 69.630 | 52.593 | 49.789 | 1.00 | 65.51 | |
| ATOM | 2009 | O1P | ADE | 94 | 70.404 | 52.063 | 50.948 | 1.00 | 65.46 | |
| ATOM | 2010 | O2P | ADE | 94 | 69.146 | 54.011 | 49.790 | 1.00 | 66.18 | |
| ATOM | 2011 | O5′ | ADE | 94 | 70.443 | 52.372 | 48.430 | 1.00 | 64.50 | |
| ATOM | 2012 | C5′ | ADE | 94 | 71.228 | 51.211 | 48.196 | 1.00 | 64.02 | |
| ATOM | 2013 | C4′ | ADE | 94 | 72.114 | 51.432 | 46.988 | 1.00 | 65.24 | |
| ATOM | 2014 | O4′ | ADE | 94 | 71.258 | 51.549 | 45.833 | 1.00 | 64.94 | |
| ATOM | 2015 | C1′ | ADE | 94 | 71.789 | 52.487 | 44.927 | 1.00 | 64.48 | |
| ATOM | 2016 | N9 | ADE | 94 | 70.781 | 53.527 | 44.738 | 1.00 | 62.23 | |
| ATOM | 2017 | C4 | ADE | 94 | 70.436 | 54.140 | 43.555 | 1.00 | 60.89 | |
| ATOM | 2018 | N3 | ADE | 94 | 70.939 | 53.905 | 42.328 | 1.00 | 60.57 | |
| ATOM | 2019 | C2 | ADE | 94 | 70.372 | 54.715 | 41.425 | 1.00 | 60.30 | |
| ATOM | 2020 | N1 | ADE | 94 | 69.434 | 55.653 | 41.607 | 1.00 | 59.12 | |
| ATOM | 2021 | C6 | ADE | 94 | 68.963 | 55.857 | 42.859 | 1.00 | 58.92 | |
| ATOM | 2022 | N6 | ADE | 94 | 68.058 | 56.803 | 43.063 | 1.00 | 59.62 | |
| ATOM | 2023 | C5 | ADE | 94 | 69.467 | 55.069 | 43.886 | 1.00 | 59.56 | |
| ATOM | 2024 | N7 | ADE | 94 | 69.185 | 55.028 | 45.241 | 1.00 | 60.03 | |
| ATOM | 2025 | C8 | ADE | 94 | 69.989 | 54.100 | 45.701 | 1.00 | 61.80 | |
| ATOM | 2026 | C2′ | ADE | 94 | 73.128 | 52.977 | 45.495 | 1.00 | 66.31 | |
| ATOM | 2027 | O2′ | ADE | 94 | 74.237 | 52.248 | 44.963 | 1.00 | 68.43 | |
| ATOM | 2028 | C3′ | ADE | 94 | 72.943 | 52.714 | 46.979 | 1.00 | 65.98 | |
| ATOM | 2029 | O3′ | ADE | 94 | 74.226 | 52.525 | 47.585 | 1.00 | 66.98 | |
| ATOM | 2030 | IR | IRI | 201 | 53.554 | 56.817 | −0.651 | 1.00 | 93.89 | |
| ATOM | 2031 | N1 | IRI | 201 | 52.182 | 58.075 | −1.938 | 1.00 | 93.42 | |
| ATOM | 2032 | N2 | IRI | 201 | 53.802 | 58.505 | 0.777 | 1.00 | 93.21 | |
| ATOM | 2033 | N3 | IRI | 201 | 54.908 | 55.460 | 0.552 | 1.00 | 94.09 | |
| ATOM | 2034 | N4 | IRI | 201 | 53.310 | 55.134 | −2.151 | 1.00 | 93.69 | |
| ATOM | 2035 | N5 | IRI | 201 | 51.753 | 56.080 | 0.473 | 1.00 | 93.32 | |
| ATOM | 2036 | N6 | IRI | 201 | 55.385 | 57.497 | −1.746 | 1.00 | 93.59 | |
| ATOM | 2037 | IR | IRI | 202 | 53.625 | 69.093 | 10.763 | 1.00 | 85.85 | |
| ATOM | 2038 | N1 | IRI | 202 | 52.087 | 70.413 | 9.778 | 1.00 | 85.66 | |
| ATOM | 2039 | N2 | IRI | 202 | 53.399 | 70.206 | 12.681 | 1.00 | 85.98 | |
| ATOM | 2040 | N3 | IRI | 202 | 55.160 | 67.721 | 11.697 | 1.00 | 84.58 | |
| ATOM | 2041 | N4 | IRI | 202 | 53.842 | 67.986 | 8.809 | 1.00 | 85.72 | |
| ATOM | 2042 | N5 | IRI | 202 | 51.949 | 67.717 | 11.391 | 1.00 | 85.26 | |
| ATOM | 2043 | N6 | IRI | 202 | 55.268 | 70.465 | 10.114 | 1.00 | 85.66 | |
| ATOM | 2044 | IR | IRI | 203 | 61.806 | 45.927 | 13.616 | 1.00 | 88.54 | |
| ATOM | 2045 | N1 | IRI | 203 | 61.031 | 48.049 | 13.783 | 1.00 | 89.40 | |
| ATOM | 2046 | N2 | IRI | 203 | 60.336 | 45.210 | 15.141 | 1.00 | 89.42 | |
| ATOM | 2047 | N3 | IRI | 203 | 62.638 | 43.838 | 13.368 | 1.00 | 89.77 | |
| ATOM | 2048 | N4 | IRI | 203 | 63.284 | 46.635 | 12.057 | 1.00 | 88.78 | |
| ATOM | 2049 | N5 | IRI | 203 | 60.295 | 45.522 | 11.996 | 1.00 | 89.15 | |
| ATOM | 2050 | N6 | IRI | 203 | 63.307 | 46.287 | 15.236 | 1.00 | 89.17 | |
| ATOM | 2051 | IR | IRI | 204 | 58.660 | 49.443 | 35.319 | 1.00 | 50.78 | |
| ATOM | 2052 | N1 | IRI | 204 | 57.805 | 49.869 | 33.303 | 1.00 | 53.99 | |
| ATOM | 2053 | N2 | IRI | 204 | 56.738 | 49.927 | 36.321 | 1.00 | 52.39 | |
| ATOM | 2054 | N3 | IRI | 204 | 59.592 | 49.024 | 37.340 | 1.00 | 54.34 | |
| ATOM | 2055 | N4 | IRI | 204 | 60.565 | 48.944 | 34.315 | 1.00 | 52.56 | |
| ATOM | 2056 | N5 | IRI | 204 | 58.146 | 47.272 | 35.175 | 1.00 | 53.83 | |
| ATOM | 2057 | N6 | IRI | 204 | 59.288 | 51.571 | 35.562 | 1.00 | 55.19 | |
| ATOM | 2058 | OH2 | TIP | 485 | 51.739 | 71.316 | 25.471 | 1.00 | 46.30 | |
| ATOM | 2059 | MG + 2 | MG2 | 302 | 44.558 | 51.948 | 20.995 | 1.00 | 53.79 | |
| ATOM | 2060 | OH2 | TIP | 486 | 49.722 | 29.399 | 20.192 | 1.00 | 54.76 | |
| ATOM | 2061 | OH2 | TIP | 487 | 39.670 | 50.220 | 26.095 | 1.00 | 61.74 | |
| ATOM | 2062 | OH2 | TIP | 488 | 65.301 | 68.258 | 4.116 | 1.00 | 63.32 | |
| ATOM | 2063 | MG + 2 | MG2 | 306 | 58.035 | 37.893 | 33.008 | 1.00 | 79.25 | |
| ATOM | 2064 | OH2 | TIP | 489 | 48.921 | 37.182 | 24.451 | 1.00 | 58.02 | |
| ATOM | 2065 | N | SAM | 4633 | 48.978 | 58.213 | 29.468 | 1.00 | 70.48 | |
| ATOM | 2066 | CA | SAM | 4633 | 49.017 | 57.559 | 28.161 | 1.00 | 70.71 | |
| ATOM | 2067 | C | SAM | 4633 | 47.811 | 57.997 | 27.341 | 1.00 | 71.98 | |
| ATOM | 2068 | O | SAM | 4633 | 47.672 | 57.591 | 26.201 | 1.00 | 74.00 | |
| ATOM | 2069 | OXT | SAM | 4633 | 46.990 | 58.794 | 27.797 | 1.00 | 72.37 | |
| ATOM | 2070 | CB | SAM | 4633 | 49.007 | 56.031 | 28.341 | 1.00 | 69.18 | |
| ATOM | 2071 | CG | SAM | 4633 | 50.053 | 55.658 | 29.384 | 1.00 | 66.96 | |
| ATOM | 2072 | SD | SAM | 4633 | 49.983 | 53.908 | 29.936 | 1.00 | 67.12 | |
| ATOM | 2073 | CE | SAM | 4633 | 49.248 | 52.995 | 28.555 | 1.00 | 66.84 | |
| ATOM | 2074 | C5* | SAM | 4633 | 48.609 | 54.077 | 31.057 | 1.00 | 63.06 | |
| ATOM | 2075 | C4* | SAM | 4633 | 49.089 | 54.531 | 32.434 | 1.00 | 59.91 | |
| ATOM | 2076 | O4* | SAM | 4633 | 49.681 | 55.840 | 32.308 | 1.00 | 57.86 | |
| ATOM | 2077 | C3* | SAM | 4633 | 47.842 | 54.726 | 33.192 | 1.00 | 57.88 | |
| ATOM | 2078 | O3* | SAM | 4633 | 47.612 | 53.550 | 33.943 | 1.00 | 59.36 | |
| ATOM | 2079 | C2* | SAM | 4633 | 48.163 | 55.893 | 34.105 | 1.00 | 56.70 | |
| ATOM | 2080 | O2* | SAM | 4633 | 48.495 | 55.463 | 35.409 | 1.00 | 59.46 | |
| ATOM | 2081 | C1* | SAM | 4633 | 49.391 | 56.633 | 33.494 | 1.00 | 55.27 | |
| ATOM | 2082 | N9 | SAM | 4633 | 49.045 | 57.972 | 33.005 | 1.00 | 51.63 | |
| ATOM | 2083 | C8 | SAM | 4633 | 49.875 | 59.041 | 32.964 | 1.00 | 49.13 | |
| ATOM | 2084 | N7 | SAM | 4633 | 49.251 | 60.083 | 32.487 | 1.00 | 49.33 | |
| ATOM | 2085 | C5 | SAM | 4633 | 47.979 | 59.734 | 32.176 | 1.00 | 50.31 | |
| ATOM | 2086 | C6 | SAM | 4633 | 46.824 | 60.386 | 31.629 | 1.00 | 49.74 | |
| ATOM | 2087 | N6 | SAM | 4633 | 46.816 | 61.719 | 31.274 | 1.00 | 49.48 | |
| ATOM | 2088 | N1 | SAM | 4633 | 45.726 | 59.668 | 31.461 | 1.00 | 50.34 | |
| ATOM | 2089 | C2 | SAM | 4633 | 45.652 | 58.380 | 31.789 | 1.00 | 49.54 | |
| ATOM | 2090 | N3 | SAM | 4633 | 46.671 | 57.755 | 32.312 | 1.00 | 49.83 | |
| ATOM | 2091 | C4 | SAM | 4633 | 47.839 | 58.372 | 32.513 | 1.00 | 50.25 | |
| ATOM | 2092 | OH2 | TIP | 401 | 57.591 | 67.639 | 5.524 | 1.00 | 67.84 | HOH |
| ATOM | 2093 | OH2 | TIP | 402 | 65.844 | 60.703 | 3.699 | 1.00 | 95.02 | HOH |
| ATOM | 2094 | OH2 | TIP | 403 | 42.141 | 28.563 | 12.608 | 1.00 | 71.25 | HOH |
| ATOM | 2095 | OH2 | TIP | 404 | 58.828 | 32.584 | 28.726 | 1.00 | 76.38 | HOH |
| ATOM | 2096 | OH2 | TIP | 405 | 42.470 | 78.500 | 33.938 | 1.00 | 65.46 | HOH |
| ATOM | 2097 | OH2 | TIP | 406 | 38.950 | 52.732 | 26.905 | 1.00 | 50.16 | HOH |
| ATOM | 2098 | OH2 | TIP | 407 | 60.495 | 26.999 | 22.724 | 1.00 | 49.53 | HOH |
| ATOM | 2099 | OH2 | TIP | 408 | 66.956 | 61.261 | 13.329 | 1.00 | 70.52 | HOH |
| ATOM | 2100 | OH2 | TIP | 409 | 54.581 | 44.687 | 21.836 | 1.00 | 48.89 | HOH |
| ATOM | 2101 | OH2 | TIP | 410 | 44.746 | 72.284 | 12.109 | 1.00 | 55.74 | HOH |
| ATOM | 2102 | OH2 | TIP | 411 | 41.215 | 64.623 | 43.219 | 1.00 | 73.60 | HOH |
| ATOM | 2103 | OH2 | TIP | 412 | 53.716 | 40.580 | 10.559 | 1.00 | 59.05 | HOH |
| ATOM | 2104 | OH2 | TIP | 413 | 59.951 | 37.704 | 24.777 | 1.00 | 66.80 | HOH |
| ATOM | 2105 | OH2 | TIP | 415 | 51.273 | 54.322 | 20.276 | 1.00 | 63.37 | HOH |
| ATOM | 2106 | OH2 | TIP | 416 | 37.853 | 76.117 | 41.745 | 1.00 | 99.78 | HOH |
| ATOM | 2107 | OH2 | TIP | 417 | 53.179 | 38.183 | 12.315 | 1.00 | 73.56 | HOH |
| ATOM | 2108 | OH2 | TIP | 418 | 68.733 | 75.988 | 9.076 | 1.00 | 92.29 | HOH |
| ATOM | 2109 | OH2 | TIP | 419 | 58.212 | 36.803 | 16.351 | 1.00 | 58.80 | HOH |
| ATOM | 2110 | OH2 | TIP | 420 | 51.901 | 53.232 | 16.541 | 1.00 | 55.72 | HOH |
| ATOM | 2111 | OH2 | TIP | 421 | 46.416 | 42.000 | 39.001 | 1.00 | 74.52 | HOH |
| ATOM | 2112 | OH2 | TIP | 422 | 40.169 | 47.590 | 33.707 | 1.00 | 84.18 | HOH |
| ATOM | 2113 | OH2 | TIP | 423 | 55.341 | 30.516 | 22.476 | 1.00 | 156.00 | HOH |
| ATOM | 2114 | OH2 | TIP | 424 | 62.161 | 39.189 | 35.898 | 1.00 | 90.20 | HOH |
| ATOM | 2115 | OH2 | TIP | 425 | 52.797 | 29.045 | 10.246 | 1.00 | 70.31 | HOH |
| ATOM | 2116 | OH2 | TIP | 426 | 46.083 | 72.202 | 14.724 | 1.00 | 89.63 | HOH |
| ATOM | 2117 | OH2 | TIP | 427 | 43.895 | 24.483 | 14.003 | 1.00 | 94.83 | HOH |
| ATOM | 2118 | OH2 | TIP | 428 | 64.042 | 51.756 | 7.838 | 1.00 | 94.93 | HOH |
| ATOM | 2119 | OH2 | TIP | 429 | 62.436 | 42.733 | 34.126 | 1.00 | 64.70 | HOH |
| ATOM | 2120 | OH2 | TIP | 430 | 41.282 | 42.563 | 19.692 | 1.00 | 87.83 | HOH |
| ATOM | 2121 | OH2 | TIP | 431 | 51.722 | 28.440 | 29.839 | 1.00 | 84.32 | HOH |
| ATOM | 2122 | OH2 | TIP | 432 | 56.273 | 41.983 | 42.811 | 1.00 | 75.94 | HOH |
| ATOM | 2123 | OH2 | TIP | 433 | 70.950 | 70.381 | 6.009 | 1.00 | 107.54 | HOH |
| ATOM | 2124 | OH2 | TIP | 434 | 43.999 | 36.134 | 19.108 | 1.00 | 59.50 | HOH |
| ATOM | 2125 | OH2 | TIP | 435 | 43.366 | 79.824 | 37.802 | 1.00 | 91.07 | HOH |
| ATOM | 2126 | OH2 | TIP | 436 | 56.922 | 36.378 | 23.973 | 1.00 | 61.98 | HOH |
| ATOM | 2127 | OH2 | TIP | 437 | 50.863 | 54.791 | 35.358 | 1.00 | 99.14 | HOH |
| ATOM | 2128 | OH2 | TIP | 438 | 42.619 | 37.414 | 16.099 | 1.00 | 97.96 | HOH |
| ATOM | 2129 | OH2 | TIP | 439 | 52.071 | 24.753 | 7.814 | 1.00 | 76.96 | HOH |
| ATOM | 2130 | OH2 | TIP | 440 | 44.787 | 70.103 | 16.931 | 1.00 | 60.20 | HOH |
| ATOM | 2131 | OH2 | TIP | 441 | 42.346 | 49.622 | 26.359 | 1.00 | 72.15 | HOH |
| ATOM | 2132 | OH2 | TIP | 442 | 43.472 | 46.657 | 12.316 | 1.00 | 90.65 | HOH |
| ATOM | 2133 | OH2 | TIP | 443 | 56.988 | 56.781 | 10.994 | 1.00 | 57.28 | HOH |
| ATOM | 2134 | OH2 | TIP | 444 | 41.847 | 29.243 | 24.223 | 1.00 | 70.54 | HOH |
| ATOM | 2135 | OH2 | TIP | 445 | 66.234 | 68.063 | 9.499 | 1.00 | 99.73 | HOH |
| ATOM | 2136 | OH2 | TIP | 446 | 44.722 | 44.081 | 37.578 | 1.00 | 62.79 | HOH |
| ATOM | 2137 | OH2 | TIP | 447 | 56.138 | 29.423 | 20.395 | 1.00 | 86.90 | HOH |
| ATOM | 2138 | OH2 | TIP | 448 | 57.334 | 59.211 | −2.014 | 1.00 | 100.61 | HOH |
| ATOM | 2139 | OH2 | TIP | 449 | 59.137 | 59.655 | 31.382 | 1.00 | 68.16 | HOH |
| ATOM | 2140 | OH2 | TIP | 450 | 42.746 | 15.877 | 21.799 | 1.00 | 92.93 | HOH |
| ATOM | 2141 | OH2 | TIP | 451 | 42.596 | 49.176 | 14.838 | 1.00 | 76.32 | HOH |
| ATOM | 2142 | OH2 | TIP | 452 | 70.669 | 48.131 | 38.000 | 1.00 | 90.22 | HOH |
| ATOM | 2143 | OH2 | TIP | 453 | 61.839 | 64.252 | 22.057 | 1.00 | 87.42 | HOH |
| ATOM | 2144 | OH2 | TIP | 454 | 47.453 | 51.753 | 25.474 | 1.00 | 79.75 | HOH |
| ATOM | 2145 | OH2 | TIP | 455 | 54.702 | 46.214 | 19.429 | 1.00 | 58.14 | HOH |
| ATOM | 2146 | OH2 | TIP | 456 | 38.160 | 46.739 | 20.938 | 1.00 | 67.56 | HOH |
| ATOM | 2147 | OH2 | TIP | 457 | 61.061 | 36.001 | 31.494 | 1.00 | 101.80 | HOH |
| ATOM | 2148 | OH2 | TIP | 458 | 44.977 | 54.138 | 34.897 | 1.00 | 83.87 | HOH |
| ATOM | 2149 | OH2 | TIP | 459 | 60.010 | 55.418 | 19.665 | 1.00 | 61.11 | HOH |
| ATOM | 2150 | OH2 | TIP | 460 | 40.810 | 49.621 | 31.081 | 1.00 | 78.05 | HOH |
| ATOM | 2151 | OH2 | TIP | 461 | 48.584 | 73.015 | 7.165 | 1.00 | 121.06 | HOH |
| ATOM | 2152 | OH2 | TIP | 462 | 39.329 | 32.085 | 23.252 | 1.00 | 88.90 | HOH |
| ATOM | 2153 | OH2 | TIP | 463 | 44.796 | 67.103 | 17.850 | 1.00 | 64.87 | HOH |
| ATOM | 2154 | OH2 | TIP | 464 | 60.056 | 60.238 | 28.863 | 1.00 | 95.98 | HOH |
| ATOM | 2155 | OH2 | TIP | 465 | 47.116 | 63.996 | 49.111 | 1.00 | 88.59 | HOH |
| ATOM | 2156 | OH2 | TIP | 466 | 41.752 | 47.571 | 27.734 | 1.00 | 60.60 | HOH |
| ATOM | 2157 | OH2 | TIP | 467 | 55.220 | 41.411 | 33.367 | 1.00 | 85.36 | HOH |
| ATOM | 2158 | OH2 | TIP | 468 | 46.419 | 34.729 | 34.906 | 1.00 | 101.53 | HOH |
| ATOM | 2159 | OH2 | TIP | 469 | 50.094 | 44.653 | 11.195 | 1.00 | 82.72 | HOH |
| ATOM | 2160 | OH2 | TIP | 470 | 54.910 | 24.423 | 9.210 | 1.00 | 69.52 | HOH |
| ATOM | 2161 | OH2 | TIP | 471 | 59.146 | 74.355 | 4.977 | 1.00 | 91.07 | HOH |
| ATOM | 2162 | OH2 | TIP | 472 | 51.468 | 40.328 | 41.242 | 1.00 | 85.30 | HOH |
| ATOM | 2163 | OH2 | TIP | 473 | 66.233 | 42.338 | 35.321 | 1.00 | 108.12 | HOH |
| ATOM | 2164 | OH2 | TIP | 474 | 55.690 | 63.785 | 36.962 | 1.00 | 92.70 | HOH |
| ATOM | 2165 | OH2 | TIP | 475 | 57.139 | 52.985 | 35.810 | 1.00 | 46.44 | HOH |
| ATOM | 2166 | OH2 | TIP | 476 | 48.166 | 19.757 | 7.460 | 1.00 | 100.21 | HOH |
| ATOM | 2167 | OH2 | TIP | 477 | 61.591 | 73.629 | 4.085 | 1.00 | 85.16 | HOH |
| ATOM | 2168 | OH2 | TIP | 478 | 57.898 | 68.572 | −1.694 | 1.00 | 70.21 | HOH |
| ATOM | 2169 | OH2 | TIP | 479 | 41.290 | 44.965 | 20.298 | 1.00 | 92.57 | HOH |
| ATOM | 2170 | OH2 | TIP | 480 | 55.825 | 58.833 | 22.615 | 1.00 | 105.63 | HOH |
| ATOM | 2171 | OH2 | TIP | 481 | 53.569 | 43.394 | 45.890 | 1.00 | 115.00 | HOH |
| ATOM | 2172 | OH2 | TIP | 482 | 58.863 | 65.274 | 17.791 | 1.00 | 65.42 | HOH |
| ATOM | 2173 | OH2 | TIP | 483 | 61.288 | 41.859 | 40.100 | 1.00 | 83.23 | HOH |
| ATOM | 2174 | OH2 | TIP | 484 | 48.702 | 33.156 | 17.506 | 1.00 | 92.02 | HOH |
| END | | | | | | | | | | |

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for identifying a compound that associates with a SAM-I riboswitch comprising:
modeling at least a portion of the SAM-I riboswitch atomic structure depicted in at least one of FIG. 2A or FIG. 2B with a test compound; and
determining the association between the test compound and the SAM-I riboswitch.

2. The method of claim 1, further comprising identifying the test compound that associates with the SAM-I riboswitch and reduces bacterial gene expression.

3. The method of claim 1, further comprising identifying the test compound that associates with the SAM-I riboswitch and induces bacterial gene expression.

4. The method of claim 1, wherein atomic coordinates of the atomic structure comprise at least a portion of the atomic coordinates listed in Table 1 for atoms depicted in FIG. 2A or 2B.

5. The method of claim 1, wherein said association determination step comprises determining a minimum interaction energy, a binding constant, a dissociation constant, or a combination thereof, for the test compound in the model of the SAM-I riboswitch.

6. The method of claim 1, wherein said association determination step comprises determining the interaction of the test compound with a nucleotide of SAM-I riboswitch comprising A6, U6, G11, A45, C47, U57, G58, A86, U87, or a combination thereof.

7. The method of claim 1, wherein said association determination step comprises determining the interaction of the test compound with a S-adenosyl-methionine moiety comprising a ribose sugar, a methionine side chain, a sulfur, an adenine moiety or combination thereof.

8. The method of claim 1, wherein said association determination step comprises determining the interaction of the test compound with a nucleotide of SAM-I riboswitch comprising A45, G11, C44, G58 and U57 or a combination thereof.

9. The method of claim 1, wherein said association determination step comprises determining the interaction of the test compound with a P3 helix region of the SAM-I riboswitch.

10. The method of claim 1, wherein said association determination step comprises determining the interaction of the test compound within a pocket created between a P1 and P3 helices of the SAM-I riboswitch.

11. The method of claim 1, wherein said association determination step comprises determining the interaction of the test compound with a minor groove faces of a P1 and P3 helices of the SAM-I riboswitch.

12. The method of claim 1, wherein the test compound reduces formation of an antiterminator conformation of the SAM-I riboswitch.

13. A method of regulating gene expression in a cell by modulating an mRNA, said method comprising administering a SAM-I riboswitch modulating compound to the cell to modulate the SAM-I riboswitch activity of the mRNA.

14. The method of claim 13, wherein the gene expression is stimulated.

15. The method of claim 13, wherein the gene expression is inhibited.

16. The method of claim 13, wherein the SAM-I riboswitch modulating compound forms a complex with the SAM-I riboswitch preventing the mRNA from forming an antiterminator element.

17. The method of claim 13, wherein the cell is a bacterial cell.

18. The method of claim 13, wherein the bacterial cell is selected from the group consisting of Staphylococcus spp., Bacillus spp., Listeria spp., Clostridia spp., Streptomyces spp., Thermoanaerobacteria spp. and a combination thereof.

19. A SAM-I riboswitch, wherein one or more of the nucleotides listed in “Tertiary contacts” section of Table 2 is modified.

20. The SAM-I riboswitch of claim 19, wherein one or more modified nucleotides are selected from the group consisting of A45, G11, C44, G58 and U57.

21. The method of claim 19, wherein the modified nucleotide increases gene expression in a cell.

22. The method of claim 19, wherein the modified nucleotide decreases gene expression in a cell.

23. The method of claim 19, wherein the modified nucleotide decreases sulfur production in a cell.

24. A composition comprising a compound that associates with at least a portion of the SAM-I riboswitch atomic structure depicted in at least one of FIG. 2A or FIG. 2B and the association includes at least one of nucleotides A45, G11, C44, G58 and U57, wherein the composition is capable of modifying the SAM-I riboswitch activity of a bacterial organism.

25. The composition of claim 24, wherein the composition further comprises a pharmaceutically acceptable excipient.

26. A composition comprising all of the 80 percent or more conserved nucleotides of the SAM-I riboswitch core depicted in FIG. 1 left and 80% or greater of the nucleotides depicted outside of the conserved region depicted in FIG. 2A or 2B.

27. The composition of claim 26, further comprising the entire atomic structure depicted in FIG. 2A or 2B.


SEQUENCE LISTING (3 sequences)

SEQ ID NO: 1 - DNA, 30 bp, Escherichia coli
gcgcgcgaattctaatacgactcactatag

SEQ ID NO: 2 - RNA, 131 nt, Bacillus subtilis
uuaaaaucucuuaucaagagagguggagggacuggcccgaugaaacccggcaaccagccuuagggcuggugccaauuccugcagcgguuucgcugaaagaugagagauucuuguagucucuucuuuuagcg

SEQ ID NO: 3 - RNA, 94 nt, Artificial sequence
  synthetic RNA
ggcuuaucaagagagguggagggacuggcccgaugaaacccggcaaccagaaauggugccaauuccugcagcggaaacguugaaagaugagcca